(19) United States
(12) Patent Application Publication
(10) Pub. No.: US 2025/0268869 A1
(43) Pub. Date: Aug. 28, 2025

(54) METHODS OF USING A (THIAZOLYL)BENZENESULFONAMIDE DERIVATIVE

(71) Applicant: Cyteir Therapeutics, Inc., Stoneham, MA (US)

(72) Inventors: Matthew Alan BELMONTE, Hollis, NH (US); William D. BRADLEY, Belmont, MA (US); Jean-Marc LAPIERRE, Merrimack, NH (US); Casey Cameron MCCOMAS, Phoenixville, PA (US); John Paul SECRIST, Southborough, MA (US); Joseph VACCA, Telford, PA (US)

(21) Appl. No.: 18/859,639

(22) PCT Filed: Apr. 25, 2023
(86) PCT No.: PCT/US2023/019820

§ 371 Date: Oct. 24, 2024

Related U.S. Application Data
(60) Provisional application No. 63/334,591, filed on Apr. 25, 2022.

(51) Int. Cl. A61K 31/427; A61P 35/00; G01N 33/58

(52) U.S. Cl. CPC A61K 31/427; A61P 35/00; G01N 33/582

(57) ABSTRACT

This application is directed to inhibitors of represented by the following structural formula, and methods for their use in modulating MCT in a disease or disorder, such as cancer or neurodegenerative diseases.

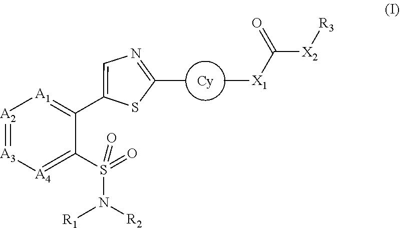

### CROSS-REFERENCES TO RELATED APPLICATIONS

This application claims priority to, and the benefit of, U.S. Provisional Application No. 63/334,591, filed on Apr. 25, 2022, the entire contents of which are incorporated herein by reference.

### BACKGROUND

Monocarboxylate transporters (MCTs) mediate influx and efflux of monocarboxylates such as lactate, pyruvate, ketone bodies (acetoacetate and beta-hydroxybutyrate) across cell membranes. These monocarboxylates play an essential role in carbohydrate, amino acid, and fat metabolism in mammalian cells. MCTs catalyze the transport of solutes via a facilitative diffusion mechanism that requires co-transport of protons. Monocarboxylates such as lactate, pyruvate, and ketone bodies play a central role in cellular metabolism and metabolic communications among tissues. Lactate is the end product of aerobic glycolysis. Lactate has recently emerged as a critical regulator of cancer development, invasion, and metastasis. Tumor lactate levels correlate well with metastasis, tumor recurrence, and poor prognosis.

MCTs are 12-span transmembrane proteins with cytosolic N- and C-termini, and are members of solute carrier SLC16A gene family. MCT family contains 14 members (e.g., MCT1, MCT2, MCT3, and MCT4 perform the function of transporting lactate, pyruvate, and ketone bodies).

Malignant tumors contain well oxygenated and hypoxic regions, and this hypoxia is associated with increased risk of cancer invasion and metastasis. Tumor hypoxia is associated with treatment failure, relapse, and patient mortality as these hypoxic cells are generally resistant to standard chemotherapy and radiation therapy. In tumors, cancer cells often prefer to utilize glycolysis rather than oxidative phosphorylation to generate energy by metabolizing glucose into lactate, and are thus referred to as glycolytic tumors. In order to avoid lactate-induced cytotoxicity, glycolytic cancer cells upregulate the expression of MCTs to increase their export capacity and avoid reaching toxic intracellular levels of lactate. In addition, nearby cancer cells have been shown to consume this lactate via MCT1 and utilize it for energy production in place of glucose.

The disclosure arises from a need to provide further compounds for the modulation of monocarboxylate transporters (MCTs). In particular, compounds with improved physicochemical, pharmacological and pharmaceutical properties to existing compounds are desirable.

### SUMMARY

The present disclosure provides methods of using a compound of Formula I:

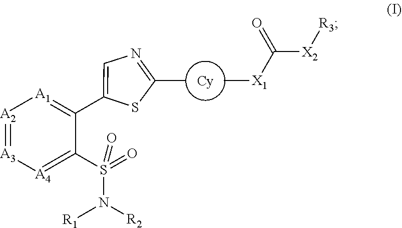

or a pharmaceutically acceptable prodrug, solvate, or salt thereof, wherein the definition of each variable is provided herein below.

The present application also provides methods of using a pharmaceutical composition comprising a compound disclosed herein or a pharmaceutically acceptable prodrug, solvate, or salt thereof and a pharmaceutically acceptable carrier or diluent.

The present disclosure further provides a method of treating a disease or disorder with implicated MCT activity.

Also provided is the use of a compound disclosed herein, or a pharmaceutically acceptable prodrug, solvate, or salt thereof, or a pharmaceutical composition disclosed herein for the preparation of a medicament for the treatment of a disease or disorder with implicated MCT activity.

Also provided is a compound disclosed herein, or a pharmaceutically acceptable prodrug, solvate, or salt thereof, or a pharmaceutical composition disclosed herein for use in treating a disease or disorder with implicated MCT activity.

In some embodiments, the disease or disorder is a cancer or a neurodegenerative disease. The method comprises administering to a subject in need thereof an effective amount of a compound disclosed herein or a pharmaceutically acceptable prodrug, solvate, or salt thereof or a pharmaceutical composition disclosed herein.

The present application provides compounds and compositions with an improved efficacy and safety profile relative to known MCT modulators. The present application also provides agents with novel mechanisms of action toward MCT in the treatment of various types of diseases. Ultimately the present application provides a novel therapeutic strategy for the treatment of diseases and disorders associated with MCT.

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### DETAILED DESCRIPTION

The present disclosure provides a compound that functions as modulator of MCT activity. The present disclosure therefore provides a method of modulating MCT activity in vitro or in vivo, said method comprising contacting a cell with an effective amount of a compound, or a pharmaceutically acceptable prodrug, solvate, or salt thereof, as defined herein.

In some aspects, the present disclosure provides a method of treating or preventing a disease or disorder associated with the abnormal expression or activity of monocarboxylate transporters (MCTs), or dependency on the expression or activity of at least one MCT, wherein the method comprises administering to a subject in need thereof a compound or a pharmaceutically acceptable prodrug, solvate, or salt thereof of the present disclosure, or a pharmaceutical composition thereof.

In some aspects, the present disclosure provides a method of treating or preventing a disease or disorder, wherein the method comprises administering to a subject in need thereof a compound or a pharmaceutically acceptable prodrug, solvate, or salt thereof of the present disclosure, or a pharmaceutical composition thereof, and
wherein the compound is administered in a therapeutically effective amount to modulate the activity of monocarboxylate transporters (MCTs).

In some aspects, the present disclosure provides a method of treating or preventing a disease or disorder, wherein the method comprises:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some aspects, the present disclosure provides the use of a compound or a pharmaceutically acceptable prodrug, solvate, or salt thereof of the present disclosure, or a pharmaceutical composition thereof, in the manufacture of a medicament for treating or preventing a disease or disorder associated with the abnormal expression or activity of monocarboxylate transporters (MCTs), or dependency on the expression or activity of at least one MCT.

In some aspects, the present disclosure provides the use of a compound or a pharmaceutically acceptable prodrug, solvate, or salt thereof of the present disclosure, or a pharmaceutical composition thereof, in the manufacture of a medicament for treating or preventing a disease or disorder, and
wherein the compound is administered in a therapeutically effective amount to modulate the activity of monocarboxylate transporters (MCTs).

In some aspects, the present disclosure provides the use of a compound or a pharmaceutically acceptable prodrug, solvate, or salt thereof of the present disclosure, or a pharmaceutical composition thereof, in the manufacture of a medicament for treating or preventing a disease or disorder comprising:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some aspects, the present disclosure provides the use of a compound or a pharmaceutically acceptable prodrug, solvate, or salt thereof of the present disclosure, or a pharmaceutical composition thereof, for treating or preventing a disease or disorder associated with the abnormal expression or activity of monocarboxylate transporters (MCTs), or dependency on the expression or activity of at least one MCT.

In some aspects, the present disclosure provides the use of a compound or a pharmaceutically acceptable prodrug, solvate, or salt thereof of the present disclosure, or a pharmaceutical composition thereof, for treating or preventing a disease or disorder, and
wherein the compound is administered in a therapeutically effective amount to modulate the activity of monocarboxylate transporters (MCTs).

In some aspects, the present disclosure provides the use of a compound or a pharmaceutically acceptable prodrug, solvate, or salt thereof of the present disclosure, or a pharmaceutical composition, for treating or preventing a disease or disorder comprising:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some aspects, the present disclosure provides a compound or a pharmaceutically acceptable prodrug, solvate, or salt thereof of the present disclosure, or a pharmaceutical composition thereof, for use in treating or preventing a disease or disorder associated with the abnormal expression or activity of monocarboxylate transporters (MCTs), or dependency on the expression or activity of at least one MCT.

In some aspects, the present disclosure provides a compound or a pharmaceutically acceptable prodrug, solvate, or salt thereof of the present disclosure, or a pharmaceutical composition thereof, for use in treating or preventing a disease or disorder, and
wherein the compound is administered in a therapeutically effective amount to modulate the activity of monocarboxylate transporters (MCTs).

In some aspects, the present disclosure provides a compound or a pharmaceutically acceptable prodrug, solvate, or salt thereof of the present disclosure, or a pharmaceutical composition thereof, for use in treating or preventing a disease or disorder comprising:
a. identifying a subject with an abnormal expression or activity of at least one MCT, or dependency on the expression or activity of at least one MCT; and
b. administering to the subject a compound of a compound or a pharmaceutically acceptable prodrug, solvate, or salt thereof of the present disclosure, or a pharmaceutical composition thereof.

In some aspects, the present disclosure provides a method of modulating MCT (e.g., the MCT1) activity (e.g., in vitro or in vivo), comprising contacting a cell with an effective amount of a compound of the present disclosure or a pharmaceutically acceptable prodrug, solvate, or salt thereof.

In some aspects, the present disclosure provides a method of modulating MCT (e.g., the MCT1) activity (e.g., in vitro or in vivo), comprising contacting a cell with a compound of the present disclosure or a pharmaceutically acceptable prodrug, solvate, or salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable prodrug, solvate, or salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable prodrug, solvate, or salt thereof, or a pharmaceutical composition of the present disclosure.

In some embodiments, the disease or disorder is associated with an implicated MCT activity. In some embodiments, the disease or disorder is a disease or disorder in which MCT activity is implicated.

In some embodiments, the disease or disorder is associated with an implicated MCT1 activity. In some embodiments, the disease or disorder is a disease or disorder in which MCT1 activity is implicated.

In some embodiments, the disease or disorder is associated with an implicated MCT4 activity. In some embodiments, the disease or disorder is a disease or disorder in which MCT4 activity is implicated.

In some embodiments, the disease or disorder is a cancer or a neurodegenerative disease.

In some aspects, the present disclosure provides a method of treating or preventing a cancer or a neurodegenerative disease in a subject in need thereof, comprising administering to the subject a therapeutically effective amount of a compound of the present disclosure or a pharmaceutically acceptable prodrug, solvate, or salt thereof, or a pharmaceutical composition of the present disclosure.

In some aspects, the present disclosure provides a method of treating or preventing a cancer or a neurodegenerative disease in a subject in need thereof, comprising administering to the subject a compound of the present disclosure or a pharmaceutically acceptable prodrug, solvate, or salt thereof, or a pharmaceutical composition of the present disclosure.

In some aspects, the present disclosure provides a method of treating a cancer or a neurodegenerative disease in a subject in need thereof, comprising administering to the subject a therapeutically effective amount of a compound of the present disclosure or a pharmaceutically acceptable prodrug, solvate, or salt thereof, or a pharmaceutical composition of the present disclosure.

In some aspects, the present disclosure provides a method of treating a cancer or a neurodegenerative disease in a subject in need thereof, comprising administering to the subject a compound of the present disclosure or a pharmaceutically acceptable prodrug, solvate, or salt thereof, or a pharmaceutical composition of the present disclosure.

In some aspects, the present disclosure provides a compound of the present disclosure or a pharmaceutically acceptable prodrug, solvate, or salt thereof for use in modulating MCT (e.g., the MCT1) activity (e.g., in vitro or in vivo).

In some aspects, the present disclosure provides a compound of the present disclosure or a pharmaceutically acceptable prodrug, solvate, or salt thereof for use in treating or preventing a disease or disorder disclosed herein.

In some aspects, the present disclosure provides a compound of the present disclosure or a pharmaceutically acceptable prodrug, solvate, or salt thereof for use in treating a disease or disorder disclosed herein.

In some aspects, the present disclosure provides a compound of the present disclosure or a pharmaceutically acceptable prodrug, solvate, or salt thereof for use in treating or preventing a cancer or a neurodegenerative disease in a subject in need thereof.

In some aspects, the present disclosure provides a compound of the present disclosure or a pharmaceutically acceptable prodrug, solvate, or salt thereof for use in treating a cancer or a neurodegenerative disease in a subject in need thereof.

In some aspects, the present disclosure provides use of a compound of the present disclosure or a pharmaceutically acceptable prodrug, solvate, or salt thereof in the manufacture of a medicament for modulating MCT (e.g., the MCT1) activity (e.g., in vitro or in vivo).

In some aspects, the present disclosure provides use of a compound of the present disclosure or a pharmaceutically acceptable prodrug, solvate, or salt thereof in the manufacture of a medicament for treating or preventing a disease or disorder disclosed herein.

In some aspects, the present disclosure provides use of a compound of the present disclosure or a pharmaceutically acceptable prodrug, solvate, or salt thereof in the manufacture of a medicament for treating a disease or disorder disclosed herein.

In some aspects, the present disclosure provides use of a compound of the present disclosure or a pharmaceutically acceptable prodrug, solvate, or salt thereof in the manufacture of a medicament for treating or preventing a cancer or a neurodegenerative disease in a subject in need thereof.

In some aspects, the present disclosure provides use of a compound of the present disclosure or a pharmaceutically acceptable prodrug, solvate, or salt thereof in the manufacture of a medicament for treating a cancer or a neurodegenerative disease in a subject in need thereof.

In some embodiments, the MCT is MCT1.

In some embodiments, the MCT is MCT4.

In some embodiments, the expression or activity of the MCT is increased.

In some embodiments, the expression or activity of the MCT is decreased.

In some embodiments, the expression or activity of MCT1 is increased.

In some embodiments, the expression or activity of MCT4 is decreased.

In some embodiments, the MCT activity of a compound or a pharmaceutically acceptable prodrug, solvate, or salt thereof of the present disclosure is assessed using a lactate transporter assay.

In some embodiments, the MCT activity of a compound or a pharmaceutically acceptable prodrug, solvate, or salt thereof of the present disclosure is assessed using an intracellular lactate accumulation assay.

In some embodiments, the MCT activity of a compound or a pharmaceutically acceptable prodrug, solvate, or salt thereof of the present disclosure is assessed using a MCT1 Transport Inhibition Assay.

In some embodiments, the disease or disorder is cancer.

In some embodiments, the cancer is a MCT1 high-expressing cancer.

In some embodiments, the cancer is a lymphoma or a solid tumor.

In some embodiments, the disease or disorder is a cancer or a neurodegenerative disease.

In some embodiments, the cancer to be treated is a B-cell neoplasm.

In some embodiments, the cancer is selected from the group consisting of lymphoma, leukemia, and a plasma cell neoplasm. In some embodiments, the cancer selected from the group consisting of carcinoma and sarcoma.

In some embodiments, the cancer to be treated is a lymphoma. Lymphomas which can be treated by the disclosed methods include Non-Hodgkin's lymphoma; Burkitt's lymphoma; small lymphocytic lymphoma; lymphoplasmacytic lymphoma; MALT lymphoma; follicular lymphoma; diffuse large B-cell lymphoma; and T-cell lymphoma.

In some embodiments, leukemias which can be treated by the disclosed methods include acute lymphoblastic leukemia (ALL); Burkitt's leukemia; B-cell leukemia; B-cell acute lymphoblastic leukemia; chronic lymphocytic leukemia (CLL); acute myelogenous leukemia (AML); chronic myelogenous leukemia (CML); and T-cell acute lymphoblastic leukemia (T-ALL).

In some embodiments the cancer to be treated is B-cell neoplasms, B-cell leukemia, B-cell acute lymphoblastic leukemia, chronic lymphocytic leukemia, chronic myelogenous leukemia, Burkitt's leukemia, acute myelogenous leukemia and/or T-ALL. In some embodiments the cancer to be treated is chronic lymphocytic leukemia (CLL) or chronic myelogenous leukemia (CML).

In some embodiments, the cancer to be treated is a plasma cell neoplasm. Examples for plasma cell neoplasms include multiple myeloma; plasma cell myeloma; plasma cell leukemia and plasmacytoma.

Carcinomas which can be treated by the disclosed methods include colon cancer; liver cancer; gastric cancer; intestinal cancer; esophageal cancer; breast cancer; ovarian cancer; head and neck cancer; lung cancer; and thyroid cancer.

Sarcomas which can be treated by the disclosed methods include soft tissue sarcoma and bone sarcoma.

In some embodiments, the cancer that can be treated by the disclosed methods include cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; sarcomas; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the disease or disorder is Lynch syndrome.

Lynch syndrome is a hereditary disorder caused by a mutation in a mismatch repair gene in which affected individuals have a higher than normal chance of developing colorectal cancer, endometrial cancer, and various other types of aggressive cancers, often at a young age—also called hereditary nonpolyposis colon cancer (HNPCC). The mutations of specific mismatch repair (MMR) genes including but not limited to MLH1, MSH2, MSH6, PMS2, and EPCAM-TACSTD1 deletions are responsible for Lynch syndrome. These genes work in repairing mistakes made when DNA is copied in preparation for cell division. The defects in the genes disallow repair of DNA mistakes and as cells divide, errors stack and uncontrollable cell growth may result in cancer. Those with Lynch syndrome carry up to an 85% risk of contracting colon cancer as well as a higher than average risk for endometrial cancer, stomach cancer, pancreatic cancer, kidney/ureter tract cancer, hepatobiliary tract cancer, gastric tract cancer, prostate cancer, ovarian cancer, gallbladder duct cancer, brain cancer, small intestine cancer, breast cancer, and skin cancer.

Thus, in one embodiment for the disclosed method, the method is a method of treating cancer derived from Lynch syndrome, selected from the group consisting of colon cancer, endometrial cancer, stomach cancer, pancreatic cancer, kidney/ureter tract cancer, hepatobiliary tract cancer, gastric tract cancer, prostate cancer, ovarian cancer, gallbladder duct cancer, brain cancer, small intestine cancer, breast cancer, and skin cancer.

In some embodiments, the neurodegenerative disorder is selected from the group consisting of multiple sclerosis, Parkinson's disease (PD), Alzheimer's disease (AD), Dentatorubropallidoluysian atrophy (DRPLA), Huntington's Disease (HD), Spinocerebellar ataxia Type 1 (SCA1), Spinocerebellar ataxia Type 2 (SCA2), Spinocerebellar ataxia Type 3 (SCA3), Spinocerebellar ataxia 6 (SCA6), Spinocerebellar ataxia Type 7 (SCA7), Spinocerebellar ataxia Type 8 (SCA8), Spinocerebellar ataxia Type 12 (SCA12), Spinocerebellar ataxia Type 17 (SCA17), Spinobulbar Muscular Ataxia/Kennedy Disease (SBMA), Fargile X syndrome (FRAXA), Fragile XE mental retardation (FRAXE), and Myotonic dystrophy (DM).

In some embodiments, the compound of the present disclosure inhibits the activity of MCT1 with an IC₅₀ from about 5 nM to about 1000 nM.

In some embodiments, the compound of the present disclosure inhibits the activity of MCT1 with an IC₅₀ of about 5 nM.

In some embodiments, the compound of the present disclosure inhibits the activity of MCT1 with an IC₅₀ of about 10 nM.

In some embodiments, the compound of the present disclosure inhibits the activity of MCT1 with an IC₅₀ of about 20 nM.

In some embodiments, the compound of the present disclosure inhibits the activity of MCT1 with an IC₅₀ of about 30 nM.

In some embodiments, the compound of the present disclosure inhibits the activity of MCT1 with an IC₅₀ of about 40 nM.

In some embodiments, the compound of the present disclosure inhibits the activity of MCT1 with an IC₅₀ of about 50 nM.

In some embodiments, the compound of the present disclosure inhibits the activity of MCT1 with an IC₅₀ of about 100 nM.

In some embodiments, the compound of the present disclosure inhibits the activity of MCT1 with an IC₅₀ of about 150 nM.

In some embodiments, the compound of the present disclosure inhibits the activity of MCT1 with an IC₅₀ of about 200 nM.

In some embodiments, the compound of the present disclosure inhibits the activity of MCT1 with an IC₅₀ of about 250 nM.

In some embodiments, the compound of the present disclosure inhibits the activity of MCT1 with an IC₅₀ of about 300 nM.

In some embodiments, the compound of the present disclosure inhibits the activity of MCT1 with an IC₅₀ of about 350 nM.

In some embodiments, the compound of the present disclosure inhibits the activity of MCT1 with an IC₅₀ of about 400 nM.

In some embodiments, the compound of the present disclosure inhibits the activity of MCT1 with an IC₅₀ of about 450 nM.

In some embodiments, the compound of the present disclosure inhibits the activity of MCT1 with an IC₅₀ of about 500 nM.

In some embodiments, the compound of the present disclosure inhibits the activity of MCT1 with an IC₅₀ of about 550 nM.

In some embodiments, the compound of the present disclosure inhibits the activity of MCT1 with an IC₅₀ of about 600 nM.

In some embodiments, the compound of the present disclosure inhibits the activity of MCT1 with an IC₅₀ of about 650 nM.

In some embodiments, the compound of the present disclosure inhibits the activity of MCT1 with an IC₅₀ of about 700 nM.

In some embodiments, the compound of the present disclosure inhibits the activity of MCT1 with an IC₅₀ of about 750 nM.

In some embodiments, the compound of the present disclosure inhibits the activity of MCT1 with an IC₅₀ of about 800 nM.

In some embodiments, the compound of the present disclosure inhibits the activity of MCT1 with an IC₅₀ of about 850 nM.

In some embodiments, the compound of the present disclosure inhibits the activity of MCT1 with an IC₅₀ of about 900 nM.

In some embodiments, the compound of the present disclosure inhibits the activity of MCT1 with an IC₅₀ of about 950 nM.

In some embodiments, the compound of the present disclosure inhibits the activity of MCT1 with an IC₅₀ of about 1000 nM.

In some embodiments, the intracellular lactate accumulation with compounds of the present disclosure has an EC₅₀ from about 5 nM to about 1000 nM.

In some embodiments, the intracellular lactate accumulation with compounds of the present disclosure has an EC₅₀ of about 5 nM.

In some embodiments, the intracellular lactate accumulation with compounds of the present disclosure has an EC₅₀ of about 10 nM.

In some embodiments, the intracellular lactate accumulation with compounds of the present disclosure has an EC₅₀ of about 20 nM.

In some embodiments, the intracellular lactate accumulation with compounds of the present disclosure has an EC₅₀ of about 30 nM.

In some embodiments, the intracellular lactate accumulation with compounds of the present disclosure has an EC₅₀ of about 40 nM.

In some embodiments, the intracellular lactate accumulation with compounds of the present disclosure has an EC₅₀ of about 50 nM.

In some embodiments, the intracellular lactate accumulation with compounds of the present disclosure has an EC₅₀ of about 100 nM.

In some embodiments, the intracellular lactate accumulation with compounds of the present disclosure has an EC₅₀ of about 150 nM.

In some embodiments, the intracellular lactate accumulation with compounds of the present disclosure has an EC₅₀ of about 200 nM.

In some embodiments, the intracellular lactate accumulation with compounds of the present disclosure has an EC₅₀ of about 250 nM.

In some embodiments, the intracellular lactate accumulation with compounds of the present disclosure has an EC₅₀ of about 300 nM.

In some embodiments, the intracellular lactate accumulation with compounds of the present disclosure has an EC₅₀ of about 350 nM.

In some embodiments, the intracellular lactate accumulation with compounds of the present disclosure has an EC₅₀ of about 400 nM.

In some embodiments, the intracellular lactate accumulation with compounds of the present disclosure has an EC₅₀ of about 450 nM.

In some embodiments, the intracellular lactate accumulation with compounds of the present disclosure has an EC₅₀ of about 500 nM.

In some embodiments, the intracellular lactate accumulation with compounds of the present disclosure has an EC₅₀ of about 550 nM.

In some embodiments, the intracellular lactate accumulation with compounds of the present disclosure has an EC₅₀ of about 600 nM.

In some embodiments, the intracellular lactate accumulation with compounds of the present disclosure has an EC₅₀ of about 650 nM.

In some embodiments, the intracellular lactate accumulation with compounds of the present disclosure has an EC₅₀ of about 700 nM.

In some embodiments, the intracellular lactate accumulation with compounds of the present disclosure has an EC₅₀ of about 750 nM.

In some embodiments, the intracellular lactate accumulation with compounds of the present disclosure has an EC₅₀ of about 800 nM.

In some embodiments, the intracellular lactate accumulation with compounds of the present disclosure has an EC₅₀ of about 850 nM.

In some embodiments, the intracellular lactate accumulation with compounds of the present disclosure has an EC₅₀ of about 900 nM.

In some embodiments, the intracellular lactate accumulation with compounds of the present disclosure has an EC₅₀ of about 950 nM.

In some embodiments, the intracellular lactate accumulation with compounds of the present disclosure has an EC₅₀ of about 1000 nM.

Effectiveness of compounds of the disclosure can be determined by industry-accepted assays/disease models according to standard practices of elucidating the same as described in the art and are found in the current general knowledge.

The present disclosure also provides a method of treating a disease or disorder in which MCT activity is implicated in a patient in need of such treatment, said method comprising administering to said patient a therapeutically effective amount of a compound, or a pharmaceutically acceptable prodrug, solvate, or salt thereof, or a pharmaceutical composition as defined herein.

Suitably, the compounds according to the present disclosure can be used for the treatment of a disease selected from a cancer or a neurodegenerative disease.

Compounds of the present disclosure, or pharmaceutically acceptable prodrugs, solvates, or salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which MCT activity is implicated comprising a compound of the disclosure as defined hereinbefore, or a pharmaceutically acceptable prodrug, solvate, or salt thereof, and another suitable agent.

According to a further aspect of the disclosure there is provided a pharmaceutical composition which comprises a compound of the disclosure, or a pharmaceutically acceptable prodrug, solvate, or salt thereof, in combination with a suitable therapeutic agent, in association with a pharmaceutically acceptable diluent or carrier.

In addition to its use in therapeutic medicine, compounds of Formula (I) and pharmaceutically acceptable prodrugs, solvates, or salts thereof are also useful as pharmacological tools in the development and standardization of in vitro and in vivo test systems for the evaluation of the effects of inhibitors of MCT in laboratory animals such as dogs, rabbits, monkeys, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

### Compounds of the Application

In one aspect, the compound of the present disclosure is a compound of Formula I:

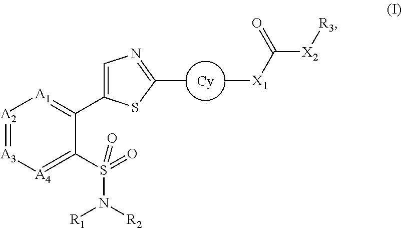

or a pharmaceutically acceptable prodrug, solvate, or salt thereof, wherein:
the thiazolyl ring

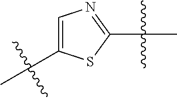 [SMILES: *c1cnc(*)s1]

is optionally substituted with F or Cl;
the ring Cy is C₃-C₇ cycloalkyl, bridged C₆-C₁₂ cycloalkyl, or saturated heterocyclyl comprising one or two 3- to 7-membered rings and 1-3 heteroatoms selected from N, O, and S, wherein the cycloalkyl or heterocyclyl moiety is optionally substituted with one or more groups selected from halogen, OH, CN, NH₂, C₁-C₄ alkyl, C₁-C₄ haloalkyl, C₁-C₄ alkoxy, and C₁-C₄ haloalkoxy;
X₁ is NR₈ or O, or, when X₁ is bonded to a nitrogen atom in the ring Cy, X₁ is absent;
X₂ is NR₈ or O;
R₁ is H or C₁-C₆ alkyl optionally substituted with halogen, OH, N(R₂)₂, C₁-C₆ alkoxy, or C₆-C₁₀ aryloxy;
R₂ is H or C₁-C₆ alkyl optionally substituted with halogen, OH, C₁-C₆ alkoxy, or C₆-C₁₀ aryloxy;
or R₁ and R₂, together with the nitrogen atom to which they are attached, form a heterocyclyl comprising one or two 3- to 7-membered rings and 1-3 heteroatoms selected from N, O, and S;
R₃ is C₁-C₆ alkyl optionally substituted with one or more groups selected from halogen, OH, and CN, phenyl, CH₂-phenyl, C₃-C₇ cycloalkyl, CH₂—(C₃-C₇) cycloalkyl, heterocyclyl, or CH₂-heterocyclyl, wherein the heterocyclyl comprises one or two 3- to 7-membered rings and 1-3 heteroatoms selected from N, O, and S, wherein the cycloalkyl, phenyl, or heterocyclyl moiety is optionally substituted with one or more groups selected from halogen, OH, CN, C₁-C₄ alkyl, C₁-C₄ haloalkyl, C₁-C₄ alkoxy, and C₁-C₄ haloalkoxy;
A₁, A₂, A₃, and A₄ are each independently N or C(R₄);
each R₄ is independently H, halogen, CN, OH, NR₈C(O)O(C₁-C₄)alkyl, NR₈C(O)N R₈—CH₂-phenyl, N(R₆′)₂, C₁-C₆ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each Q is independently C₁-C₄ alkylene or O—(C₁-C₄) alkylene wherein the oxygen atom is bonded to the ring

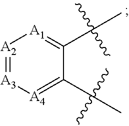

each T is independently C₁-C₄ alkoxy, OH, N(R₆)₂, N(R₅)C(═O)R₆, N(R₅)C(═O)OR₆, C(═O)N(R₆)₂, C(═O)OR₆, C(═O)R₆, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each R₅ is independently H or C₁-C₄ alkyl;
each R₆′ is independently H, C(O)NHR₇, C₁-C₆ alkyl optionally substituted with one or more R₇, C₁-C₆ haloalkyl, C₃-C₇ cycloalkyl, heterocyclyl comprising one or two 3- to 7-membered rings and 1-3 heteroatoms selected from N, O, and S, C₆-C₁₀ aryl, or heteroaryl comprising one or two 5- or 6-membered rings and 1-3 heteroatoms selected from N, O, and S, wherein the cycloalkyl, heterocyclyl, aryl, or heteroaryl moiety is optionally substituted with one or more R₉, wherein at least one R₆′ is not H; or two R₆′ together with the atoms to which they are attached form a 3- to 10-membered heterocyclyl comprising 1-3 heteroatoms selected from N, O, and S, wherein the heterocyclyl is optionally substituted with one or more R₉;
each R₆ is independently H, C₁-C₆ alkyl optionally substituted with one or more R₇, C₁-C₆ haloalkyl, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉; or two R₆ together with the atoms to which they are attached form a 3- to 10-membered heterocyclyl comprising 1-3 heteroatoms selected from N, O, and S, wherein the heterocyclyl is optionally substituted with one or more R₉;
each R₇ is independently N(R₈)₂, OR₈, C₆-C₁₀ aryl, or heteroaryl comprising one or two 5- or 6-membered rings and 1-3 heteroatoms selected from N, O, and S;
each R₈ is independently H or C₁-C₆ alkyl; and
each R₉ is independently oxo, halogen, OH, CN, NH₂, N(C₁-C₄ alkyl)₂, C₁-C₆ alkyl, N(C₁-C₄ alkyl)₂, C₁-C₆ haloalkyl, C₁-C₆ alkoxy, or C₁-C₆ haloalkoxy, wherein the C₁-C₆ alkyl is optionally substituted with one or more oxo, OH, O(C₁-C₄ alkyl), CN, NH₂, NH(C₁-C₄ alkyl), or N(C₁-C₄ alkyl)₂.

In one aspect, the compound of the present disclosure is a compound of Formula I:

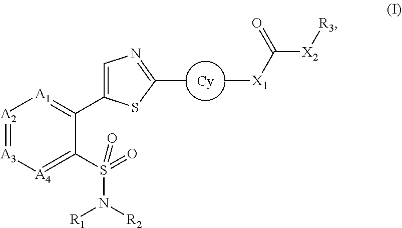

or a pharmaceutically acceptable prodrug, solvate, or salt thereof, wherein:
the thiazolyl ring

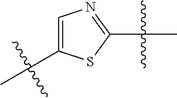 [SMILES: *c1cnc(*)s1]

is optionally substituted with F or Cl;
the ring Cy is C₃-C₇ cycloalkyl, bridged C₆-C₁₂ cycloalkyl, or saturated heterocyclyl comprising one or two 3- to 7-membered rings and 1-3 heteroatoms selected from N, O, and S, wherein the cycloalkyl or heterocyclyl moiety is optionally substituted with one or more groups selected from halogen, OH, CN, NH₂, C₁-C₄ alkyl, C₁-C₄ haloalkyl, C₁-C₄ alkoxy, and C₁-C₄ haloalkoxy;
X₁ is NR₈ or O, or, when X₁ is bonded to a nitrogen atom in the ring Cy, X₁ is absent;
X₂ is NR₈ or O;
R₁ is H or C₁-C₆ alkyl optionally substituted with halogen, OH, C₁-C₆ alkoxy, or C₆-C₁₀ aryloxy;
R₂ is H or C₁-C₆ alkyl optionally substituted with halogen, OH, C₁-C₆ alkoxy, or C₆-C₁₀ aryloxy;
or R₁ and R₂, together with the nitrogen atom to which they are attached, form a 3-7 membered heterocyclic ring comprising 1 or 2 heteroatoms selected from N, O, and S; R₃ is C₁-C₆ alkyl optionally substituted with one or more groups selected from halogen, OH, and CN, phenyl, CH₂-phenyl, C₃-C₇ cycloalkyl, CH₂—(C₃-C₇) cycloalkyl, heterocyclyl, or CH₂-heterocyclyl, wherein the heterocyclyl comprises one 3- to 7-membered ring and 1 or 2 heteroatoms selected from N, O, and S, wherein the cycloalkyl, phenyl, or heterocyclyl moiety is optionally substituted with one or more groups selected from halogen, OH, CN, C₁-C₄ alkyl, C₁-C₄ haloalkyl, C₁-C₄ alkoxy, and C₁-C₄ haloalkoxy;
A₁, A₂, A₃, and A₄ are each independently N or C(R₄);
each R₄ is independently H, halogen, CN, OH, N(R₆′)₂, C₁-C₄ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each Q is independently C₁-C₄ alkylene or O—(C₁-C₄) alkylene wherein the oxygen atom is bonded to the ring

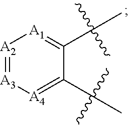

each T is independently C₁-C₄ alkoxy, NH₂, NH(C₁-C₄ alkyl), N(C₁-C₄ alkyl)₂, N(R₅)C(═O)R₆, N(R₅)C(═O)OR₆, C(═O)N(R₆)₂, C(═O)OR₆, or C(═O)R₆;
each R₅ is independently H or C₁-C₄ alkyl;
each R₆′ is independently H, C₁-C₆ alkyl optionally substituted with one or more R₇, C₁-C₆ haloalkyl, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉, wherein at least one R₆′ is not H;
each R₆ is independently H, C₁-C₆ alkyl optionally substituted with one or more R₇, C₁-C₆ haloalkyl, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each R₇ is independently N(R₈)₂ or OR₈;
each R₈ is independently H or C₁-C₆ alkyl; and
each R₉ is independently OH, CN, NH₂, N(C₁-C₄ alkyl)₂, C₁-C₆ alkyl optionally substituted with one or more groups selected from OH, CN, NH₂, and N(C₁-C₄ alkyl)₂, C₁-C₆ haloalkyl, C₁-C₆ alkoxy, or C₁-C₆ haloalkoxy.

In some embodiments, the compound of Formula I is of Formula Ia or Ib:

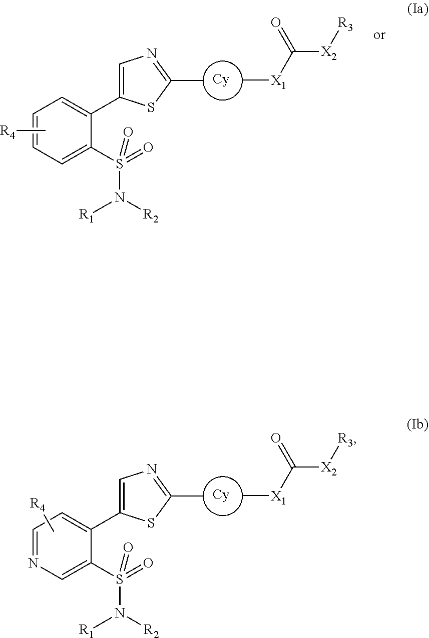

or a pharmaceutically acceptable prodrug, solvate, or salt thereof.

In some embodiments, the compound of Formula I is of Formula Ic:

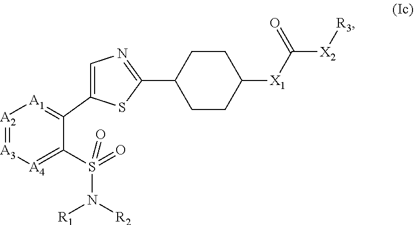

or a pharmaceutically acceptable prodrug, solvate, or salt thereof.

In some embodiments, the compound of Formula I is of Formula Id, Ie, If, or Ig:

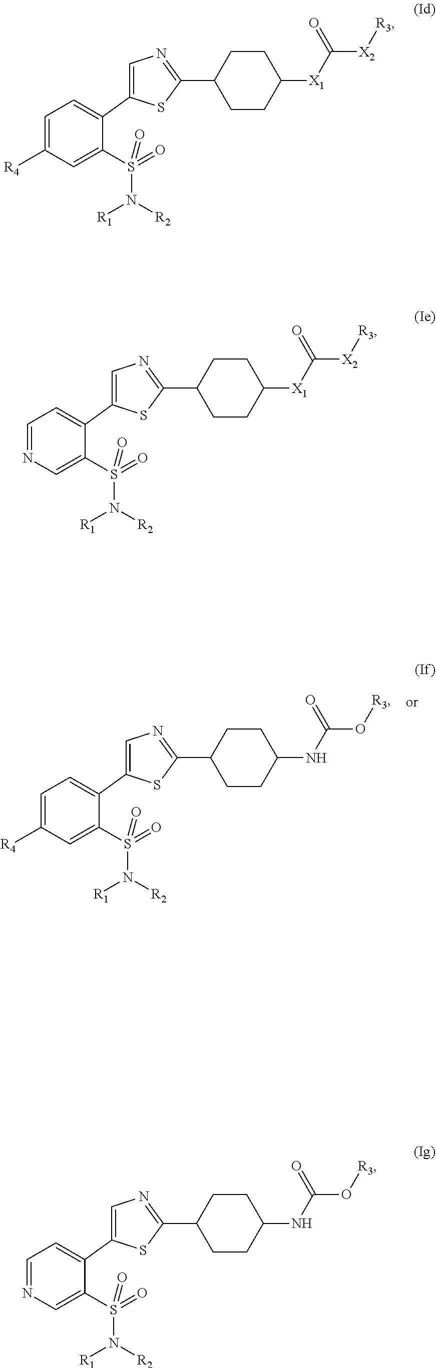

or a pharmaceutically acceptable prodrug, solvate, or salt thereof.

In some embodiments, the compound of Formula I is of Formula IIa or IIb:

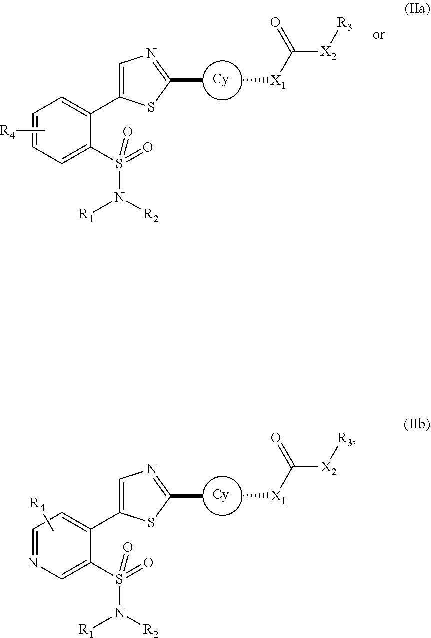

or a pharmaceutically acceptable prodrug, solvate, or salt thereof.

In some embodiments, the compound of Formula I is of Formula IIc:

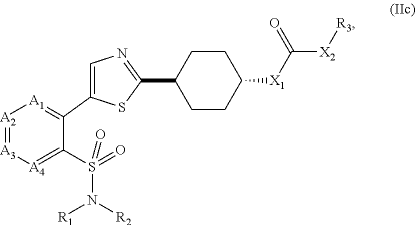

or a pharmaceutically acceptable prodrug, solvate, or salt thereof.

In some embodiments, the compound of Formula I is of Formula IId, IIe, IIf, or IIg:

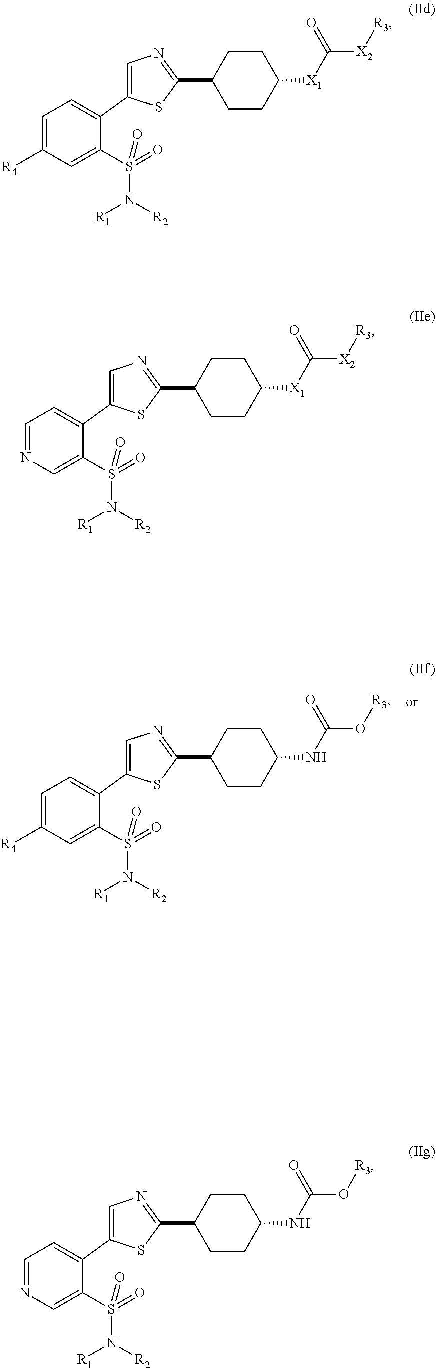

or a pharmaceutically acceptable prodrug, solvate, or salt thereof.

Fora compound of any of Formula I, Ia, Ib, Ic, Id, Ie, If, Ig, IIa, IIb, IIc, IId, IIe, IIf, or IIg, where applicable:
(A1) In some embodiments, the thiazolyl ring

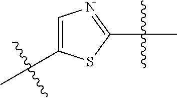 [SMILES: *c1cnc(*)s1]

is unsubstituted.
(A2) In some embodiments, the thiazolyl ring

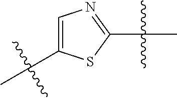 [SMILES: *c1cnc(*)s1]

is substituted with F.
(A3) In some embodiments, the thiazolyl ring

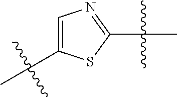 [SMILES: *c1cnc(*)s1]

is substituted with Cl.
(B1) In some embodiments, the ring Cy is C₃-C₇ cycloalkyl selected from cyclopropyl, cyclobutyl, cyclopentyl, cyclohexyl, and cycloheptyl, each of which is optionally substituted with one or more groups selected from halogen (e.g., F, Cl, Br, or I), OH, CN, NH₂, C₁-C₄ alkyl (e.g., methyl, ethyl, n-propyl, i-propyl, n-butyl, i-butyl, s-butyl, or t-butyl), C₁-C₄ haloalkyl (e.g., methyl, ethyl, n-propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, or butoxy), and C₁-C₄ haloalkoxy (e.g., methoxy, ethoxy, propoxy, or butoxy, each of which is substituted with one or more halogen (e.g., F, Cl, Br, or I)). In some embodiments, the ring Cy is cyclohexyl, optionally substituted as described herein.
(B2) In some embodiments, the ring Cy is bridged C₆-C₁₂ cycloalkyl selected from bicyclo[2.1.1]hexyl, bicyclo[2.2.1]heptyl, bicyclo[2.2.2]octyl, bicyclo[3.2.1]octyl, bicyclo[4.3.1]decyl, bicyclo[3.3.1]nonyl, bornyl, bornenyl, norbornyl, norbornenyl, 6,6-dimethylbicyclo [3.1.1]heptyl, and adamantyl, each of which is optionally substituted with one or more groups selected from halogen (e.g., F, Cl, Br, or I), OH, CN, NH₂, C₁-C₄ alkyl (e.g., methyl, ethyl, n-propyl, i-propyl, n-butyl, i-butyl, s-butyl, or t-butyl), C₁-C₄ haloalkyl (e.g., methyl, ethyl, n-propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, or butoxy), and C₁-C₄ haloalkoxy (e.g., methoxy, ethoxy, propoxy, or butoxy, each of which is substituted with one or more halogen (e.g., F, Cl, Br, or I)).
(B3) In some embodiments, the ring Cy is saturated heterocyclyl selected from aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, diazaspiro[4.4]nonyl, diazaspiro[3.5]nonyl, hexahydropyridazinyl, hexahydropyrimidinyl, tetrahydrothiopyranyl, thiomorpholinyl, tropanyl, valerolactamyl, azanorbornyl, quinuclidinyl, isoquinuclidinyl, azabicyclo[2.2.1]heptanyl, 2-azabicyclo[3.2.1]octanyl, azabicyclo[3.2.1]octanyl, azabicyclo[3.2.2]nonanyl, azabicyclo[3.3.0]nonanyl, azabicyclo[3.3.1]nonanyl, diazabicyclo[2.2.1]heptanyl, diazabicyclo[3.2.1]octanyl, octahydropyrrolo[3,4-b]pyrrolyl, and octahydropyrrolo[3,4-c]pyrrolyl, each of which is optionally substituted with one or more groups selected from halogen (e.g., F, Cl, Br, or I), OH, CN, NH₂, C₁-C₄ alkyl (e.g., methyl, ethyl, n-propyl, i-propyl, n-butyl, i-butyl, s-butyl, or t-butyl), C₁-C₄ haloalkyl (e.g., methyl, ethyl, n-propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, or butoxy), and C₁-C₄ haloalkoxy (e.g., methoxy, ethoxy, propoxy, or butoxy, each of which is substituted with one or more halogen (e.g., F, Cl, Br, or I)).
(C1) In some embodiments, X₁ is NR₈ and X₂ is NR. In a further embodiment, each R₈ is H. In another further embodiment, one R is H, and the other R is C₁-C₆ alkyl (e.g., methyl, ethyl, n-propyl, i-propyl, n-butyl, i-butyl, s-butyl, t-butyl, pentyl (straight or branched), or hexyl (straight or branched)). In another further embodiment, each R₈ is independently C₁-C₆ alkyl (e.g., methyl, ethyl, n-propyl, i-propyl, n-butyl, i-butyl, s-butyl, t-butyl, pentyl (straight or branched), or hexyl (straight or branched)).
(C2) In some embodiments, X₁ is NR₈ and X₂ is O. In a further embodiment, R₈ is H. In another further embodiment, R is C₁-C₆ alkyl (e.g., methyl, ethyl, n-propyl, i-propyl, n-butyl, i-butyl, s-butyl, t-butyl, pentyl (straight or branched), or hexyl (straight or branched)).
(C3) In some embodiments, X₁ is O and X₂ is NR₈. In a further embodiment, R₈ is H. In another further embodiment, R₈ is C₁-C₆ alkyl (e.g., methyl, ethyl, n-propyl, i-propyl, n-butyl, i-butyl, s-butyl, t-butyl, pentyl (straight or branched), or hexyl (straight or branched)).
(C4) In some embodiments, X₁ is O and X₂ is O.
(C5) In some embodiments, X₁ is absent and X₂ is NR. In a further embodiment, R₈ is H. In another further embodiment, R₈ is C₁-C₆ alkyl (e.g., methyl, ethyl, n-propyl, i-propyl, n-butyl, i-butyl, s-butyl, t-butyl, pentyl (straight or branched), or hexyl (straight or branched)).
(C6) In some embodiments, X₁ is absent and X₂ is O.
(D1) In some embodiments, R₁ is H.
(D2) In some embodiments, R₁ is C₁-C₆ alkyl selected from methyl, ethyl, n-propyl, i-propyl, n-butyl, i-butyl, s-butyl, t-butyl, pentyl (straight or branched), and hexyl (straight or branched), each of which is optionally substituted with one or more groups selected from halogen (e.g., F, Cl, Br, or I), OH, C₁-C₆ alkoxy (e.g., methoxy, ethoxy, propoxy, butoxy, pentoxy, or hexyloxy), and C₆-C₁₀ aryloxy (e.g., phenoxy). In some embodiments, R₁ is C₁-C₄ alkyl selected from methyl, ethyl, n-propyl, i-propyl, n-butyl, i-butyl, s-butyl, and t-butyl, each of which is optionally substituted with one or more groups selected from halogen (e.g., F, Cl, Br, or I), OH, C₁-C₆ alkoxy (e.g., methoxy, ethoxy, propoxy, butoxy, pentoxy, or hexyloxy), and C₆-C₁₀ aryloxy (e.g., phenoxy). In some embodiments, R₁ is C₁-C₄ alkyl selected from methyl, ethyl, n-propyl, i-propyl, n-butyl, i-butyl, s-butyl, and t-butyl, each of which is optionally substituted with one or more groups selected from halogen (e.g., F, Cl, Br, or I) and OH. In some embodiments, R₁ is unsubstituted C₁-C₆ alkyl. In some embodiments, R₁ is unsubstituted C₁-C₄ alkyl.
(E1) In some embodiments, R₂ is H.
(E2) In some embodiments, R₂ is C₁-C₆ alkyl selected from methyl, ethyl, n-propyl, i-propyl, n-butyl, i-butyl, s-butyl, t-butyl, pentyl (straight or branched), and hexyl (straight or branched), each of which is optionally substituted with one or more groups selected from halogen (e.g., F, Cl, Br, or I), OH, C₁-C₆ alkoxy (e.g., methoxy, ethoxy, propoxy, butoxy, pentoxy, or hexyloxy), and C₆-C₁₀ aryloxy (e.g., phenoxy). In some embodiments, R₂ is C₁-C₄ alkyl selected from methyl, ethyl, n-propyl, i-propyl, n-butyl, i-butyl, s-butyl, and t-butyl, each of which is optionally substituted with one or more groups selected from halogen (e.g., F, Cl, Br, or I), OH, C₁-C₆ alkoxy (e.g., methoxy, ethoxy, propoxy, butoxy, pentoxy, or hexyloxy), and C₆-C₁₀ aryloxy (e.g., phenoxy). In some embodiments, R₂ is C₁-C₄ alkyl selected from methyl, ethyl, n-propyl, i-propyl, n-butyl, i-butyl, s-butyl, and t-butyl, each of which is optionally substituted with one or more groups selected from halogen (e.g., F, Cl, Br, or I) and OH. In some embodiments, R₂ is unsubstituted C₁-C₆ alkyl. In some embodiments, R₂ is unsubstituted C₁-C₄ alkyl.
(DE) In some embodiments, R₁ and R₂, together with the nitrogen atom to which they are attached, form heterocyclic ring selected from aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, and thiomorpholinyl. In some embodiments, R₁ and R₂, together with the nitrogen atom to which they are attached, form azetidinyl or pyrrolidinyl.
(F1) In some embodiments, R₃ is C₁-C₆ alkyl selected from methyl, ethyl, n-propyl, i-propyl, n-butyl, i-butyl, s-butyl, t-butyl, pentyl (straight or branched), and hexyl (straight or branched), each of which is optionally substituted with one or more groups selected from halogen (e.g., F, Cl, Br, or I), OH, and CN. In some embodiments, R₃ is C₁-C₄ alkyl selected from methyl, ethyl, n-propyl, i-propyl, n-butyl, i-butyl, s-butyl, and t-butyl, each of which is optionally substituted with one or more groups selected from halogen (e.g., F, Cl, Br, or I), OH, and CN. In some embodiments, R₃ is unsubstituted C₁-C₆ alkyl. In some embodiments, R₃ is unsubstituted C₁-C₄ alkyl. In some embodiments, R₃ is unsubstituted methyl. In some embodiments, R₃ is unsubstituted i-propyl. In some embodiments, R₃ is unsubstituted t-butyl.
(F2) In some embodiments, R₃ is phenyl or CH₂-phenyl, wherein the phenyl moiety is optionally substituted with one or more groups selected from halogen (e.g., F, Cl, Br, or I), OH, CN, C₁-C₄ alkyl (e.g., methyl, ethyl, n-propyl, i-propyl, n-butyl, i-butyl, s-butyl, or t-butyl), C₁-C₄ haloalkyl (e.g., methyl, ethyl, n-propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, or butoxy), and C₁-C₄ haloalkoxy (e.g., methoxy, ethoxy, propoxy, or butoxy, each of which is substituted with one or more halogen (e.g., F, Cl, Br, or I)).
(F3) In some embodiments, R₃ is C₃-C₇ cycloalkyl or CH₂—(C₃-C₇) cycloalkyl, wherein the cycloalkyl moiety is selected from cyclopropyl, cyclobutyl, cyclopentyl, cyclohexyl, and cycloheptyl, each of which is optionally substituted with one or more groups selected from halogen (e.g., F, Cl, Br, or I), OH, CN, C₁-C₄ alkyl (e.g., methyl, ethyl, n-propyl, i-propyl, n-butyl, i-butyl, s-butyl, or t-butyl), C₁-C₄ haloalkyl (e.g., methyl, ethyl, n-propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, or butoxy), and C₁-C₄ haloalkoxy (e.g., methoxy, ethoxy, propoxy, or butoxy, each of which is substituted with one or more halogen (e.g., F, Cl, Br, or I)). In some embodiments, R₃ is cyclopropyl or cyclobutyl, or CH₂-cyclopropyl or CH₂-cyclobutyl, each of which is optionally substituted as described herein. In some embodiments, R₃ is cyclopropyl optionally substituted as described herein.
(F4) In some embodiments, R₃ is heterocyclyl or CH₂-heterocyclyl, wherein the heterocyclyl moiety is selected from aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, and thiomorpholinyl, each of which is optionally substituted with one or more groups selected from halogen (e.g., F, Cl, Br, or I), OH, CN, C₁-C₄ alkyl (e.g., methyl, ethyl, n-propyl, i-propyl, n-butyl, i-butyl, s-butyl, or t-butyl), C₁-C₄ haloalkyl (e.g., methyl, ethyl, n-propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, or butoxy), and C₁-C₄ haloalkoxy (e.g., methoxy, ethoxy, propoxy, or butoxy, each of which is substituted with one or more halogen (e.g., F, Cl, Br, or I)). In some embodiments, R₃ is unsubstituted oxetanyl.
(G1) In some embodiments, A₁, A₂, A₃, and A₄ are each C(R₄).
(G2) In some embodiments, one, two, or three of A₁, A₂, A₃, and A₄ are N, and the remaining of A₁, A₂, A₃, and A₄ are C(R₄).
(G3) In some embodiments, one of A₁, A₂, A₃, and A₄ is N, and the remaining of A₁, A₂, A₃, and A₄ are C(R₄).
(G3a) In some embodiments, A₁ is N, and A₂, A₃, and A₄ are C(R₄).
(G3b) In some embodiments, A₂ is N, and A₁, A₃, and A₄ are C(R₄).
(G3c) In some embodiments, A₃ is N, and A₁, A₂, and A₄ are C(R₄).
(G3d) In some embodiments, A₄ is N, and A₁, A₂, and A₃ are C(R₄).
(G4) In some embodiments, two of A₁, A₂, A₃, and A₄ are N, and the remaining of A₁, A₂, A₃, and A₄ are C(R₄).
(G4a) In some embodiments, A₁ and A₂ are N, and A₃ and A₄ are C(R₄).
(G4b) In some embodiments, A₁ and A₃ are N, and A₂ and A₄ are C(R₄).
(G4c) In some embodiments, A₁ and A₄ are N, and A₂ and A₃ are C(R₄).
(G4d) In some embodiments, A₂ and A₃ are N, and A₁ and A₄ are C(R₄).
(G4e) In some embodiments, A₂ and A₄ are N, and A₁ and A₃ are C(R₄).
(G4f) In some embodiments, A₃ and A₄ are N, and A₁ and A₂ are C(R₄).
(G5) In some embodiments, three of A₁, A₂, A₃, and A₄ are N, and the remaining of A₁, A₂, A₃, and A₄ is C(R₄).
(G5a) In some embodiments, A₂, A₃, and A₄ are N, and A₁ is C(R₄).
(G5b) In some embodiments, A₁, A₃, and A₄ are N, and A₂ is C(R₄).
(G5c) In some embodiments, A₁, A₂, and A₄ are N, and A₃ is C(R₄).
(G5d) In some embodiments, A₁, A₂, and A₃ are N, and A₄ is C(R₄).
(G6) In some embodiments, A₁, A₂, A₃, and A₄ are each N.
(G7) In some embodiments, at most two of A₁, A₂, A₃, and A₄ are N.
(H1) In some embodiments, each R₄ is H.
(H2) In some embodiments, at least one R₄ is halogen, CN, OH, N(R₆′)₂, C₁-C₆ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉.
(H2a) In some embodiments, at least one R₄ is halogen (e.g., F, Cl, Br, or I), CN, OH, N(R₆′)₂, C₁-C₆ alkoxy (e.g., methoxy, ethoxy, propoxy, butoxy, pentoxy, or hexoxy), C(═O)N(R₆)₂, C(═O)OR₆, C(═O)R₆, or Q-T.
(H2b) In some embodiments, at least one R₄ is CN, OH, N(R₆′)₂, C₁-C₆ alkoxy (e.g., methoxy, ethoxy, propoxy, butoxy, pentoxy, or hexoxy), C(═O)N(R₆)₂, C(═O)OR₆, C(═O)R₆, or Q-T.
(H2c) In some embodiments, at least one R₄ is C(═O)N(R₆)₂, C(═O)OR₆, C(═O)R₆, or Q-T.
(H2d) In some embodiments, at least one R₄ is Q-T.
(H2e) In some embodiments, at least one R₄ is N(R₆′)₂, and one of R₆′ is H, and the other R₆′ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)), C₃-C₇ cycloalkyl (e.g., cyclopropyl, cyclobutyl, cyclopentyl, cyclohexyl, or cycloheptyl), heterocyclyl comprising one or two 3- to 7-membered rings and 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), C₆-C₁₀ aryl (e.g., phenyl), or heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the cycloalkyl, heterocyclyl, aryl, or heteroaryl moiety is optionally substituted with one or more R₉.
(H2e-1) In some embodiments, at least one R₄ is N(R₆′)₂, and one of R₆′ is H, and the other R₆′ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), or C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(H2e-2) In some embodiments, at least one R₄ is N(R₆′)₂, and one of R₆′ is H, and the other R₆′ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇).
(H2e-3) In some embodiments, at least one R₄ is N(R₆′)₂, and one of R₆′ is H, and the other R₆′ is C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(H2e-4) In some embodiments, at least one R₄ is N(R₆′)₂, and one of R₆′ is H, and the other R₆′ is C₃-C₇ cycloalkyl (e.g., cyclopropyl, cyclobutyl, cyclopentyl, cyclohexyl, or cycloheptyl), wherein the cycloalkyl moiety is optionally substituted with one or more R₉.
(H2e-5) In some embodiments, at least one R₄ is N(R₆′)₂, and one of R₆′ is H, and the other R₆′ is heterocyclyl comprising one or two 3- to 7-membered rings and 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), wherein the heterocyclyl moiety is optionally substituted with one or more R₉. In some embodiments, at least one R₄ is N(R₆′)₂, and one of R₆′ is H, and the other R₆′ is heterocyclyl comprising one 4- to 6-membered ring and 1-3 heteroatoms selected from N, O, and S, as described herein, wherein the heterocyclyl moiety is optionally substituted with one or more R₉. In some embodiments, at least one R₄ is N(R₆′)₂, and one of R₆′ is H, and the other R₆′ is heterocyclyl comprising one 4- to 5-membered ring and 1-3 heteroatoms selected from N, O, and S, as described herein, wherein the heterocyclyl moiety is optionally substituted with one or more R₉. In some embodiments, at least one R₄ is N(R₆′)₂, and one of R₆′ is H, and the other R₆′ is heterocyclyl comprising one 4-membered ring and 1-2 heteroatoms selected from N, O, and S, as described herein, wherein the heterocyclyl moiety is optionally substituted with one or more R₉. In some embodiments, at least one R₄ is N(R₆′)₂, and one of R₆′ is H, and the other R₆′ is heterocyclyl comprising one 4-membered ring and 1 heteroatom selected from N, O, and S, as described herein, wherein the heterocyclyl moiety is optionally substituted with one or more R₉.
(H2e-6) In some embodiments, at least one R₄ is N(R₆′)₂, and one of R₆′ is H, and the other R₆′ is C₆-C₁₀ aryl (e.g., phenyl), wherein the aryl moiety is optionally substituted with one or more R₉.
(H2e-7) In some embodiments, at least one R₄ is N(R₆′)₂, and one of R₆′ is H, and the other R₆′ is heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, triazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the heteroaryl moiety is optionally substituted with one or more R₉. In some embodiments, at least one R₄ is N(R₆′)₂, and one of R₆′ is H, and the other R₆′ is heteroaryl comprising one 5- or 6-membered ring and 1-3 heteroatoms selected from N, O, and S, as described herein, wherein the heteroaryl moiety is optionally substituted with one or more R₉. In some embodiments, at least one R₄ is N(R₆′)₂, and one of R₆′ is H, and the other R₆′ is heteroaryl comprising one 5-membered ring and 1-3 heteroatoms selected from N, O, and S, as described herein, wherein the heteroaryl moiety is optionally substituted with one or more R₉. In some embodiments, at least one R₄ is N(R₆′)₂, and one of R₆′ is H, and the other R₆′ is heteroaryl comprising one 6-membered ring and 1-3 heteroatoms selected from N, O, and S, as described herein, wherein the heteroaryl moiety is optionally substituted with one or more R₉.
(H2e-8) In some embodiments, at least one R₄ is N(R₆′)₂, and the two R₆′ are each independently C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), or C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(H2e-9) In some embodiments, at least one R₄ is N(R₆′)₂, and the two R₆′ together with the atoms to which they are attached form a 3- to 10-membered heterocyclyl comprising 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), wherein the heterocyclyl is optionally substituted with one or more R₉. In some embodiments, at least one R₄ is N(R₆′)₂, and the two R₆′ together with the atoms to which they are attached form a 3- to 6-membered heterocyclyl comprising 1-3 heteroatoms selected from N, O, and S, as described herein, and is optionally substituted with one or more R₉. In some embodiments, at least one R₄ is N(R₆′)₂, and the two R₆′ together with the atoms to which they are attached form a 4- to 6-membered heterocyclyl comprising 1-3 heteroatoms selected from N, O, and S, as described herein, and is optionally substituted with one or more R₉. In some embodiments, at least one R₄ is N(R₆′)₂, and the two R₆′ together with the atoms to which they are attached form azetidinyl, piperidinyl, morpholinyl, or thiomorpholino, each of which is optionally substituted with one or more R₉.
(H2e-10) In some embodiments, at least one R₄ is N(R₆′)₂, and one of R₆′ is C₁-C₆ alkyl or C₁-C₆ haloalkyl, as described herein, and the other R₆′ is cycloalkyl, heterocyclyl comprising one or two 3- to 7-membered rings and 1-3 heteroatoms selected from N, O, and S, C₆-C₁₀ aryl, or heteroaryl comprising one or two 5- or 6-membered rings and 1-3 heteroatoms selected from N, O, and S, as described herein, wherein the cycloalkyl, heterocyclyl, aryl, or heteroaryl moiety is optionally substituted with one or more R₉.
(H2f) In some embodiments, at least one R₄ is C₆-C₁₀ aryl (e.g., phenyl) or heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the aryl or heteroaryl moiety is optionally substituted with one or more R₉.
(H2f-1) In some embodiments, at least one R₄ is C₆-C₁₀ aryl (e.g., phenyl), wherein the aryl moiety is optionally substituted with one or more R₉.
(H2f-2) In some embodiments, at least one R₄ is heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the heteroaryl moiety is optionally substituted with one or more R₉. In some embodiments, at least one R₄ is heteroaryl comprising one 5- or 6-membered ring and 1-3 heteroatoms selected from N, O, and S, as described herein, wherein the heteroaryl moiety is optionally substituted with one or more R₉. In some embodiments, at least one R₄ is heteroaryl comprising one 5-membered ring and 1-3 heteroatoms selected from N, O, and S, as described herein, wherein the heteroaryl moiety is optionally substituted with one or more R₉. In some embodiments, at least one R₄ is heteroaryl comprising one 6-membered ring and 1-3 heteroatoms selected from N, O, and S, as described herein, wherein the heteroaryl moiety is optionally substituted with one or more R₉.
(H3) In some embodiments, only one R₄ is halogen, CN, OH, N(R₆′)₂, C₁-C₆ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉.
(H3a) In some embodiments, only one R₄ is halogen (e.g., F, Cl, Br, or I), CN, OH, N(R₆′)₂, C₁-C₆ alkoxy (e.g., methoxy, ethoxy, propoxy, butoxy, pentoxy, or hexoxy), C(═O)N(R₆)₂, C(═O)OR₆, C(═O)R₆, or Q-T.
(H3b) In some embodiments, only one R₄ is CN, OH, N(R₆′)₂, C₁-C₆ alkoxy (e.g., methoxy, ethoxy, propoxy, butoxy, pentoxy, or hexoxy), C(═O)N(R₆)₂, C(═O)OR₆, C(═O)R₆, or Q-T.
(H3c) In some embodiments, only one R₄ is C(═O)N(R₆)₂, C(═O)OR₆, C(═O)R₆, or Q-T.
(H3d) In some embodiments, only one R₄ is Q-T.
(H3e) In some embodiments, only one R₄ is N(R₆′)₂, and one of R₆′ is H, and the other R₆′ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)), C₃-C₇ cycloalkyl, heterocyclyl comprising one or two 3- to 7-membered rings and 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), C₆-C₁₀ aryl (e.g., phenyl), or heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the cycloalkyl, heterocyclyl, aryl, or heteroaryl moiety is optionally substituted with one or more R₉.
(H3e-1) In some embodiments, only one R₄ is N(R₆′)₂, and one of R₆′ is H, and the other R₆′ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), or C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(H3e-2) In some embodiments, only one R₄ is N(R₆′)₂, and one of R₆′ is H, and the other R₆′ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇).
(H3e-3) In some embodiments, only one R₄ is N(R₆′)₂, and one of R₆′ is H, and the other R₆′ is C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(H3e-4) In some embodiments, only one R₄ is N(R₆′)₂, and one of R₆′ is H, and the other R₆′ is C₃-C₇ cycloalkyl (e.g., cyclopropyl, cyclobutyl, cyclopentyl, cyclohexyl, or cycloheptyl), wherein the cycloalkyl moiety is optionally substituted with one or more R₉.
(H3e-5) In some embodiments, only one R₄ is N(R₆′)₂, and one of R₆′ is H, and the other R₆′ is heterocyclyl comprising one or two 3- to 7-membered rings and 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), wherein the heterocyclyl moiety is optionally substituted with one or more R₉. In some embodiments, only one R₄ is N(R₆′)₂, and one of R₆′ is H, and the other R₆′ is heterocyclyl comprising one 4- to 6-membered ring and 1-3 heteroatoms selected from N, O, and S, as described herein, wherein the heterocyclyl moiety is optionally substituted with one or more R₉. In some embodiments, only one R₄ is N(R₆′)₂, and one of R₆′ is H, and the other R₆′ is heterocyclyl comprising one 4- to 5-membered ring and 1-3 heteroatoms selected from N, O, and S, as described herein, wherein the heterocyclyl moiety is optionally substituted with one or more R₉. In some embodiments, only one R₄ is N(R₆′)₂, and one of R₆′ is H, and the other R₆′ is heterocyclyl comprising one 4-membered ring and 1-2 heteroatoms selected from N, O, and S, as described herein, wherein the heterocyclyl moiety is optionally substituted with one or more R₉. In some embodiments, only one R₄ is N(R₆′)₂, and one of R₆′ is H, and the other R₆′ is heterocyclyl comprising one 4-membered ring and 1 heteroatom selected from N, O, and S, as described herein, wherein the heterocyclyl moiety is optionally substituted with one or more R₉.
(H3e-6) In some embodiments, only one R₄ is N(R₆′)₂, and one of R₆′ is H, and the other R₆′ is C₆-C₁₀ aryl (e.g., phenyl), wherein the aryl moiety is optionally substituted with one or more R₉.
(H3e-7) In some embodiments, only one R₄ is N(R₆′)₂, and one of R₆′ is H, and the other R₆′ is heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the heteroaryl moiety is optionally substituted with one or more R₉. In some embodiments, only one R₄ is N(R₆′)₂, and one of R₆′ is H, and the other R₆′ is heteroaryl comprising one 5- or 6-membered ring and 1-3 heteroatoms selected from N, O, and S, as described herein, wherein the heteroaryl moiety is optionally substituted with one or more R₉. In some embodiments, only one R₄ is N(R₆′)₂, and one of R₆′ is H, and the other R₆′ is heteroaryl comprising one 5-membered ring and 1-3 heteroatoms selected from N, O, and S, as described herein, wherein the heteroaryl moiety is optionally substituted with one or more R₉. In some embodiments, only one R₄ is N(R₆′)₂, and one of R₆′ is H, and the other R₆′ is heteroaryl comprising one 6-membered ring and 1-3 heteroatoms selected from N, O, and S, as described herein, wherein the heteroaryl moiety is optionally substituted with one or more R₉.
(H3e-8) In some embodiments, only one R₄ is N(R₆′)₂, and the two R₆′ are each independently C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), or C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(H3e-9) In some embodiments, only one R₄ is N(R₆′)₂, and the two R₆′ together with the atoms to which they are attached form a 3- to 10-membered heterocyclyl comprising 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), wherein the heterocyclyl is optionally substituted with one or more R₉. In some embodiments, only one R₄ is N(R₆′)₂, and the two R₆′ together with the atoms to which they are attached form a 3- to 6-membered heterocyclyl comprising 1-3 heteroatoms selected from N, O, and S, as described herein, and is optionally substituted with one or more R₉. In some embodiments, only one R₄ is N(R₆′)₂, and the two R₆′ together with the atoms to which they are attached form a 4- to 6-membered heterocyclyl comprising 1-3 heteroatoms selected from N, O, and S, as described herein, and is optionally substituted with one or more R₉. In some embodiments, only one R₄ is N(R₆′)₂, and the two R₆′ together with the atoms to which they are attached form azetidinyl, piperidinyl, morpholinyl, or thiomorpholino, each of which is optionally substituted with one or more R₉.
(H3e-10) In some embodiments, only one R₄ is N(R₆′)₂, and one of R₆′ is C₁-C₆ alkyl or C₁-C₆ haloalkyl, as described herein, and the other R₆′ is cycloalkyl, heterocyclyl comprising one or two 3- to 7-membered rings and 1-3 heteroatoms selected from N, O, and S, C₆-C₁₀ aryl, or heteroaryl comprising one or two 5- or 6-membered rings and 1-3 heteroatoms selected from N, O, and S, as described herein, wherein the cycloalkyl, heterocyclyl, aryl, or heteroaryl moiety is optionally substituted with one or more R₉.
(H3f) In some embodiments, only one R₄ is C₆-C₁₀ aryl (e.g., phenyl) or heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the aryl or heteroaryl moiety is optionally substituted with one or more R₉.
(H3f-1) In some embodiments, only one R₄ is C₆-C₁₀ aryl (e.g., phenyl), wherein the aryl moiety is optionally substituted with one or more R₉.
(H3f-2) In some embodiments, only one R₄ is heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the heteroaryl moiety is optionally substituted with one or more R₉. In some embodiments, only one R₄ is heteroaryl comprising one 5- or 6-membered ring and 1-3 heteroatoms selected from N, O, and S, as described herein, wherein the heteroaryl moiety is optionally substituted with one or more R₉. In some embodiments, only one R₄ is heteroaryl comprising one 5-membered ring and 1-3 heteroatoms selected from N, O, and S, as described herein, wherein the heteroaryl moiety is optionally substituted with one or more R₉. In some embodiments, only one R₄ is heteroaryl comprising one 6-membered ring and 1-3 heteroatoms selected from N, O, and S, as described herein, wherein the heteroaryl moiety is optionally substituted with one or more R₉.
(H3g) In some embodiments, at least one R₄ is C₁-C₆ alkoxy (e.g., methoxy, ethoxy, propoxy, butoxy, pentoxy, or hexoxy).
(H3g-1) In some embodiments, only one R₄ is C₁-C₆ alkoxy (e.g., methoxy, ethoxy, propoxy, butoxy, pentoxy, or hexoxy).
(I1) In some embodiments, at least one Q is C₁-C₄ alkylene (e.g., methylene, ethylene, propylene, or butylene).
(I2) In some embodiments, at least one Q is O—(C₁-C₄) alkylene (e.g., O-methylene, O-ethylene, O-propylene, or O-butylene).
(J1) In some embodiments, at least one T is C₁-C₄ alkoxy (e.g., methoxy, ethoxy, propoxy, or butoxy), OH, N(R₆)₂, N(R₅)C(═O)R₆, N(R₅)C(═O)OR₆, C(═O)N(R₆)₂, C(═O)OR₆, C(═O)R₆, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉.
(J2) In some embodiments, at least one T is C₁-C₄ alkoxy (e.g., methoxy, ethoxy, propoxy, or butoxy).
(J3) In some embodiments, at least one T is NH₂, NH(R₆), or N(R₆)₂.
(J3-1) In some embodiments, at least one T is NH₂, NH(R₆), or N(R₆)₂, wherein R₆ is C₁-C₄ alkyl moiety is selected from methyl, ethyl, n-propyl, i-propyl, n-butyl, i-butyl, s-butyl, and t-butyl.
(J4) In some embodiments, at least one T is N(R₅)C(═O)R₆ or N(R₅)C(═O)OR₆.
(J5) In some embodiments, at least one T is C(═O)N(R₆)₂, C(═O)OR₆, or C(═O)R₆.
(J6) In some embodiments, at least one T is OH.
(J7) In some embodiments, at least one T is C₆-C₁₀ aryl or heteroaryl comprising one or two 5- or 6-membered rings and 1-3 heteroatoms selected from N, O, and S, wherein the aryl or heteroaryl moiety is optionally substituted with one or more R₉.
(J7-1) In some embodiments, at least one T is C₆-C₁₀ aryl (e.g., phenyl), optionally substituted with one or more R₉.
(J7-2) In some embodiments, at least one T is heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), optionally substituted with one or more R₉. In some embodiments, at least one T is heteroaryl comprising one 5- or 6-membered ring and 1-3 heteroatoms selected from N, O, and S, as described herein, wherein the heteroaryl moiety is optionally substituted with one or more R₉. In some embodiments, at least one T is heteroaryl comprising one 5-membered ring and 1-3 heteroatoms selected from N, O, and S, as described herein, wherein the heteroaryl moiety is optionally substituted with one or more R₉. In some embodiments, at least one T is heteroaryl comprising one 6-membered ring and 1-3 heteroatoms selected from N, O, and S, as described herein, wherein the heteroaryl moiety is optionally substituted with one or more R₉.
(K1) In some embodiments, each R₅ is H.
(K2) In some embodiments, at least one R₅ is C₁-C₄ alkyl (e.g., methyl, ethyl, n-propyl, i-propyl, n-butyl, i-butyl, s-butyl, or t-butyl).
(L1) In some embodiments, each R₆ is H.
(L2) In some embodiments, at least one R₆ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)), C₆-C₁₀ aryl (e.g., phenyl), or heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the aryl or heteroaryl moiety is optionally substituted with one or more R₉.
(L2a) In some embodiments, at least one R₆ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇), or C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)).
(L2b) In some embodiments, at least one R₆ is C₁-C₆ alkyl (e.g., methyl, ethyl, n-propyl, i-propyl, n-butyl, i-butyl, s-butyl, t-butyl, pentyl (straight or branched), or hexyl (straight or branched), each of which is optionally substituted with one or more R₇).
(L2c) In some embodiments, at least one R₆ is C₆-C₁₀ aryl (e.g., phenyl), wherein the aryl moiety is optionally substituted with one or more R₉.
(L2d) In some embodiments, at least one R₆ is heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl), wherein the heteroaryl moiety is optionally substituted with one or more R₉. In some embodiments, at least one R₆ is heteroaryl comprising one 5- or 6-membered ring and 1-3 heteroatoms selected from N, O, and S, as described herein, wherein the heteroaryl moiety is optionally substituted with one or more R₉. In some embodiments, at least one R₆ is heteroaryl comprising one 5-membered ring and 1-3 heteroatoms selected from N, O, and S, as described herein, wherein the heteroaryl moiety is optionally substituted with one or more R₉. In some embodiments, at least one R₆ is heteroaryl comprising one 6-membered ring and 1-3 heteroatoms selected from N, O, and S, as described herein, wherein the heteroaryl moiety is optionally substituted with one or more R₉.
(L2e) In some embodiments, two R₆ together with the atoms to which they are attached form a 3- to 10-membered heterocyclyl comprising 1-3 heteroatoms selected from N, O, and S (e.g., aziridinyl, oxiranyl, thiiranyl, diaziridinyl, oxaziridinyl, dioxiranyl, azetidinyl, oxetanyl, thietanyl, diazetidinyl, dioxetanyl, dithietanyl, pyrrolidinyl, tetrahydrofuranyl, tetrahydrothiophenyl, imidazolidinyl, pyrazolidinyl, oxazolidinyl, isoxazolidinyl, thiazolidinyl, isothiazolidinyl, dioxolanyl, dithiolany, piperidinyl, tetrahydropyranyl, thianyl, piperazinyl, morpholinyl, thiomorpholinyl, dioxanyl, dithianyl, hexahydro-1,3,5-triazinyl, trioxanyl, trithianyl, azepanyl, oxepanyl, thiepanyl, diazepanyl, hexahydropyridazinyl, hexahydropyrimidinyl, tetrahydrothiopyranyl, or thiomorpholinyl), wherein the heterocyclyl is optionally substituted with one or more R₉. In some embodiments, two R₆ together with the atoms to which they are attached form a 3- to 6-membered heterocyclyl comprising 1-3 heteroatoms selected from N, O, and S, as described herein, and is optionally substituted with one or more R₉. In some embodiments, two R₆ together with the atoms to which they are attached form a 4- to 6-membered heterocyclyl comprising 1-3 heteroatoms selected from N, O, and S, as described herein, and is optionally substituted with one or more R₉.
(M1a) In some embodiments, each R₇ is independently N(R₈)₂, and each R₈ is H.
(M1b) In some embodiments, each R₇ is independently N(R₈)₂, and one R₈ is H, and the other R₈ is C₁-C₆ alkyl (e.g., methyl, ethyl, n-propyl, i-propyl, n-butyl, i-butyl, s-butyl, t-butyl, pentyl (straight or branched), or hexyl (straight or branched)).
(M1c) In some embodiments, each R₇ is independently N(R₈)₂, and each R₈ is independently C₁-C₆ alkyl (e.g., methyl, ethyl, n-propyl, i-propyl, n-butyl, i-butyl, s-butyl, t-butyl, pentyl (straight or branched), or hexyl (straight or branched)).
(M2a) In some embodiments, each R₇ is independently OR₈, and R₈ is H.
(M2b) In some embodiments, each R₇ is independently OR₈, and R₈ is C₁-C₆ alkyl (e.g., methyl, ethyl, n-propyl, i-propyl, n-butyl, i-butyl, s-butyl, t-butyl, pentyl (straight or branched), or hexyl (straight or branched)).
(M2c) In some embodiments, each R₇ is independently C₆-C₁₀ aryl (e.g., phenyl) or heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl).
(M2c-1) In some embodiments, each R₇ is independently C₆-C₁₀ aryl (e.g., phenyl).
(M2c-2) In some embodiments, each R₇ is independently heteroaryl comprising one or two 5- or 6-membered rings and 1-3 heteroatoms selected from N, O, and S (e.g., pyridinyl, pyrazinyl, pyrimidinyl, pyridazinyl, pyrrolyl, pyrazolyl, imidazolyl, thiazolyl, oxazolyl, isooxazolyl, isothiazolyl, thiadiazolyl, oxadiazolyl, thiophenyl, furanyl, isoquinolinyl, indolyl, pyrazolopyridinyl, indazolyl, benzofuranyl, benzoxazolyl, benzothiazolyl, or benzotriazolyl).
(N1) In some embodiments, each R₉ is independently oxo, halogen (e.g., F, Cl, Br, or I), OH, CN, NH₂, N(C₁-C₄ alkyl)₂ (e.g., dimethylamino, methylethylamino, diethylamino, methylpropylamino, ethylpropylamino, dipropylamino, methylbutylamino, ethylbutylamino, propylbutylamino, or dibutyamino), C₁-C₆ alkyl (e.g., methyl, ethyl, n-propyl, i-propyl, n-butyl, i-butyl, s-butyl, t-butyl, pentyl (straight or branched), or hexyl (straight or branched)), C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)), C₁-C₆ alkoxy (e.g., methoxy, ethoxy, propoxy, butoxy, pentoxy, or hexyloxy), or C₁-C₆ haloalkoxy (e.g., methoxy, ethoxy, propoxy, butoxy, pentoxy, or hexyloxy, each of which is substituted with one or more halogen (e.g., F, Cl, Br, or I)), wherein the C₁-C₆ alkyl is optionally substituted with one or more groups selected from oxo, OH, O(C₁-C₄ alkyl), CN, NH₂, and N(C₁-C₄ alkyl)₂ (e.g., dimethylamino, methylethylamino, diethylamino, methylpropylamino, ethylpropylamino, dipropylamino, methylbutylamino, ethylbutylamino, propylbutylamino, or dibutyamino).
(N2) In some embodiments, each R₉ is independently OH, CN, NH₂, N(C₁-C₄ alkyl)₂ (e.g., dimethylamino, methylethylamino, diethylamino, methylpropylamino, ethylpropylamino, dipropylamino, methylbutylamino, ethylbutylamino, propylbutylamino, or dibutyamino), C₁-C₆ alkyl (e.g., methyl, ethyl, n-propyl, i-propyl, n-butyl, i-butyl, s-butyl, t-butyl, pentyl (straight or branched), or hexyl (straight or branched)), C₁-C₆ haloalkyl (e.g., methyl, ethyl, n-propyl, i-propyl, n-butyl, i-butyl, s-butyl, t-butyl, pentyl (straight or branched), or hexyl (straight or branched), each of which is substituted with one or more halogen (e.g., F, Cl, Br, or I)), C₁-C₆ alkoxy (e.g., methoxy, ethoxy, propoxy, butoxy, pentoxy, or hexyloxy), or C₁-C₆ haloalkoxy (e.g., methoxy, ethoxy, propoxy, butoxy, pentoxy, or hexyloxy, each of which is substituted with one or more halogen (e.g., F, Cl, Br, or I)), wherein the C₁-C₆ alkyl is optionally substituted with one or more groups selected from OH, CN, NH₂, and N(C₁-C₄ alkyl)₂ (e.g., dimethylamino, methylethylamino, diethylamino, methylpropylamino, ethylpropylamino, dipropylamino, methylbutylamino, ethylbutylamino, propylbutylamino, or dibutyamino).

For a compound of any of Formula I, Ia, Ib, Ic, Id, Ie, If, Ig, IIa, IIb, IIc, IId, IIe, IIf, or IIg, where applicable, each of the substituent groups illustrated herein for any of Cy, X₁, X₂, A₁, A₂, A₃, A₄, R₁, R₂, R₃, R₄, R₅, R₆, R₆′, R₇, R₈, R₉, Q, and T, can be combined with any of the substituent groups illustrated herein for one or more of the remainder of Cy, X₁, X₂, A₁, A₂, A₃, A₄, R₁, R₂, R₃, R₄, R₅, R₆, R₆′, R₇, R₈, R₉, Q, and T.

For example:
(O1) The thiazolyl ring

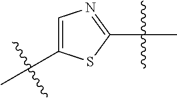 [SMILES: *c1cnc(*)s1]

is as illustrated in any of (A1)-(A3), and the Cy ring is as illustrated in (B1).
(O2) The thiazolyl ring

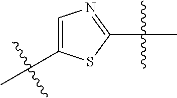 [SMILES: *c1cnc(*)s1]

is as illustrated in any of (A1)-(A3), and the Cy ring is as illustrated in (B2).
(O3) The thiazolyl ring

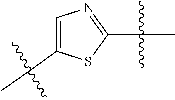 [SMILES: *c1cnc(*)s1]

is as illustrated in any of (A1)-(A3), and the Cy ring is as illustrated in (B3).
(P1) The thiazolyl ring

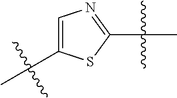 [SMILES: *c1cnc(*)s1]

is as illustrated in any of (A1)-(A3), and A₁, A₂, A₃, and A₄ are each as illustrated in (G1).
(P2) The thiazolyl ring

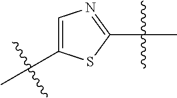 [SMILES: *c1cnc(*)s1]

is as illustrated in any of (A1)-(A3), and A₁, A₂, A₃, and A₄ are each as illustrated in (G2).
(P3) The thiazolyl ring

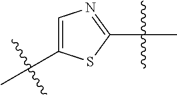 [SMILES: *c1cnc(*)s1]

is as illustrated in any of (A1)-(A3), and A₁, A₂, A₃, and A₄ are each as illustrated in any of (G3)-(G3d).
(P4) The thiazolyl ring

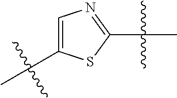 [SMILES: *c1cnc(*)s1]

is as illustrated in any of (A1)-(A3), and A₁, A₂, A₃, and A₄ are each as illustrated in any of (G4)-(G4f).
(P5) The thiazolyl ring

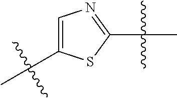 [SMILES: *c1cnc(*)s1]

is as illustrated in any of (A1)-(A3), and A₁, A₂, A₃, and A₄ are each as illustrated in any of (G5)-(G5d).
(P6) The thiazolyl ring

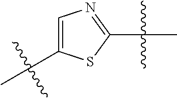 [SMILES: *c1cnc(*)s1]

is as illustrated in any of (A1)-(A3), and A₁, A₂, A₃, and A₄ are each as illustrated in any of (G6).
(P7) The thiazolyl ring

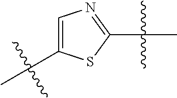 [SMILES: *c1cnc(*)s1]

is as illustrated in any of (A1)-(A3), and A₁, A₂, A₃, and A₄ are each as illustrated in any of (G7).
(Q1a) The Cy ring is as illustrated in (B1), and A₁, A₂, A₃, and A₄ are each as illustrated in (G1).
(Q1b) The Cy ring is as illustrated in (B1), and A₁, A₂, A₃, and A₄ are each as illustrated in (G2).
(Q1c) The Cy ring is as illustrated in (B1), and A₁, A₂, A₃, and A₄ are each as illustrated in any of (G3)-(G3d).
(Q1d) The Cy ring is as illustrated in (B1), and A₁, A₂, A₃, and A₄ are each as illustrated in any of (G4)-(G4f).
(Q1e) The Cy ring is as illustrated in (B1), and A₁, A₂, A₃, and A₄ are each as illustrated in any of (G5)-(G5d).
(Q1f) The Cy ring is as illustrated in (B1), and A₁, A₂, A₃, and A₄ are each as illustrated in any of (G6).
(Q1g) The Cy ring is as illustrated in (B1), and A₁, A₂, A₃, and A₄ are each as illustrated in any of (G7).
(Q2a) The Cy ring is as illustrated in (B2), and A₁, A₂, A₃, and A₄ are each as illustrated in (G1).
(Q2b) The Cy ring is as illustrated in (B2), and A₁, A₂, A₃, and A₄ are each as illustrated in (G2).
(Q2c) The Cy ring is as illustrated in (B2), and A₁, A₂, A₃, and A₄ are each as illustrated in any of (G3)-(G3d).
(Q2d) The Cy ring is as illustrated in (B2), and A₁, A₂, A₃, and A₄ are each as illustrated in any of (G4)-(G4f).
(Q2e) The Cy ring is as illustrated in (B2), and A₁, A₂, A₃, and A₄ are each as illustrated in any of (G5)-(G5d).
(Q2f) The Cy ring is as illustrated in (B2), and A₁, A₂, A₃, and A₄ are each as illustrated in any of (G6).
(Q2g) The Cy ring is as illustrated in (B2), and A₁, A₂, A₃, and A₄ are each as illustrated in any of (G7).
(Q3a) The Cy ring is as illustrated in (B3), and A₁, A₂, A₃, and A₄ are each as illustrated in (G1).
(Q3b) The Cy ring is as illustrated in (B3), and A₁, A₂, A₃, and A₄ are each as illustrated in (G2).
(Q3c) The Cy ring is as illustrated in (B3), and A₁, A₂, A₃, and A₄ are each as illustrated in any of (G3)-(G3d).
(Q3d) The Cy ring is as illustrated in (B3), and A₁, A₂, A₃, and A₄ are each as illustrated in any of (G4)-(G4f).
(Q3e) The Cy ring is as illustrated in (B3), and A₁, A₂, A₃, and A₄ are each as illustrated in any of (G5)-(G5d).
(Q3f) The Cy ring is as illustrated in (B3), and A₁, A₂, A₃, and A₄ are each as illustrated in any of (G6).
(Q3g) The Cy ring is as illustrated in (B3), and A₁, A₂, A₃, and A₄ are each as illustrated in any of (G7).
(Q4a) The thiazolyl ring

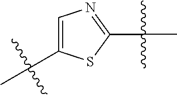 [SMILES: *c1cnc(*)s1]

and the Cy ring are illustrated as in any of (O1)-(O3), and A₁, A₂, A₃, and A₄ are each as illustrated in (G1).
(Q4b) The thiazolyl ring

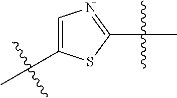 [SMILES: *c1cnc(*)s1]

and the Cy ring are illustrated as in any of (O1)-(O3), and A₁, A₂, A₃, and A₄ are each as illustrated in (G2).
(Q4c) The thiazolyl ring

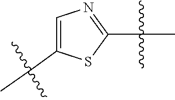 [SMILES: *c1cnc(*)s1]

and the Cy ring are illustrated as in any of (O1)-(O3), and A₁, A₂, A₃, and A₄ are each as illustrated in any of (G3)-(G3d).
(Q4d) The thiazolyl ring

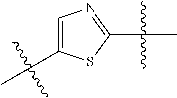 [SMILES: *c1cnc(*)s1]

and the Cy ring are illustrated as in any of (O1)-(O3), and A₁, A₂, A₃, and A₄ are each as illustrated in any of (G4)-(G4f).
(Q4e) The thiazolyl ring

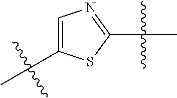 [SMILES: *c1cnc(*)s1]

and the Cy ring are illustrated as in any of (O1)-(O3), and A₁, A₂, A₃, and A₄ are each as illustrated in any of (G5)-(G5d).
(Q4f) The thiazolyl ring

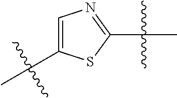 [SMILES: *c1cnc(*)s1]

and the Cy ring are illustrated as in any of (O1)-(O3), and A₁, A₂, A₃, and A₄ are each as illustrated in any of (G6).
(Q4g) The thiazolyl ring

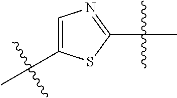 [SMILES: *c1cnc(*)s1]

and the Cy ring are illustrated as in any of (O1)-(O3), and A₁, A₂, A₃, and A₄ are each as illustrated in any of (G7).
(R1) The thiazolyl ring

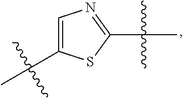 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H1).
(R2) The thiazolyl ring

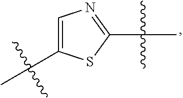 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H2).
(R3) The thiazolyl ring

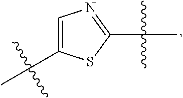 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H2a).
(R4) The thiazolyl ring

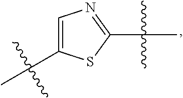 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H2b).
(R5) The thiazolyl ring

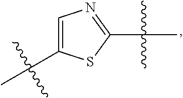 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H2c).
(R6) The thiazolyl ring

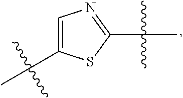 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H2d).
(R7) The thiazolyl ring

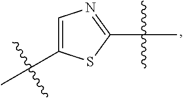 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in any of (H2e)-(H2e-10).
(R8) The thiazolyl ring

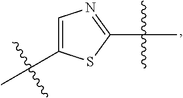 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H2f)-(H2f-2).
(R9) The thiazolyl ring

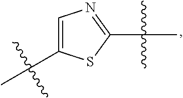 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3).
(R10) The thiazolyl ring

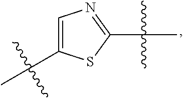 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3a).
(R11) The thiazolyl ring

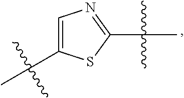 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3b).
(R12) The thiazolyl ring

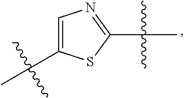 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3c).
(R13) The thiazolyl ring

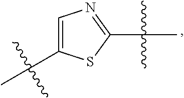 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3d).
(R14) The thiazolyl ring

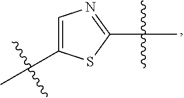 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in any of (H3e)-(H3e-10).
(R15) The thiazolyl ring

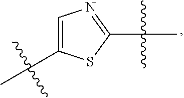 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3f-1)-(H3f-2).
(R16) The thiazolyl ring

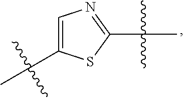 [SMILES: *c1cnc(*)s1]

the Cy ring, and/or A₁, A₂, A₃, and A₄ are each as illustrated, as applicable, in any or (A1)-(A3), (B1)-(B3), (G1)-(G7), and (O1)-(Q4g), R₄ is as illustrated in (H3g)-(H3g-1).

Also included are the compounds disclosed in the Exemplification, both in the pharmaceutically acceptable salt form and in the neutral form.

In some embodiments, the compound is selected from the compounds described in Table 1 and pharmaceutically acceptable prodrugs, solvates, or salts thereof.

In some embodiments, the compound is selected from the compounds described in Table 1.

Non-limiting illustrative compounds of the application are listed in Table 1.

TABLE 1

| Cmpd | | |
|---|---|---|
| No | Structure | Chemical Name |
| 1 | 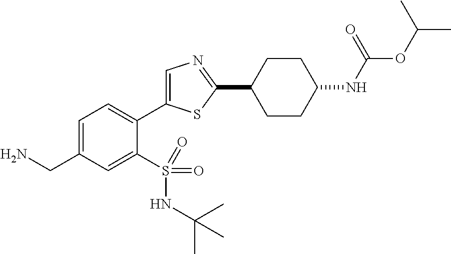 | isopropyl (trans-4-(5-(4- (aminomethyl)-2-(N-(tert- butyl)sulfamoyl)phenyl)thiazol-2- yl)cyclohexyl)carbamate |
| 2 | 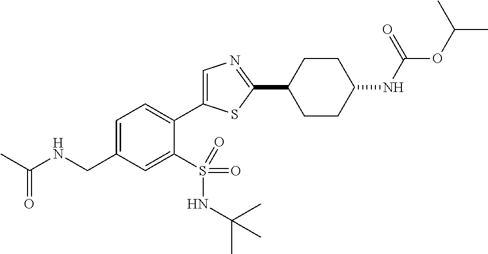 | isopropyl (trans-4-(5-(4- (acetamidomethyl)-2-(N-(tert- butyl)sulfamoyl)phenyl)thiazol-2- yl)cyclohexyl)carbamate |
| 3 | 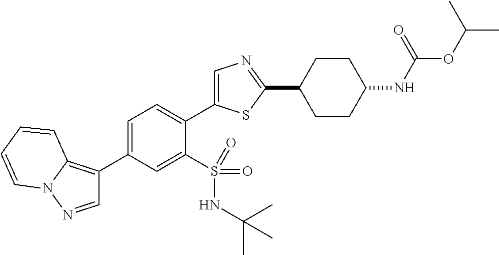 | isopropyl (trans-4-(5-(2-(N-(tert- butyl)sulfamoyl)-4-(pyrazolo[1,5- a]pyridin-3-yl)phenyl)thiazol-2- yl)cyclohexyl)carbamate |
| 4 | 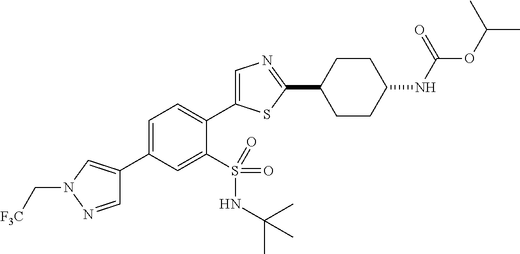 | isopropyl (trans-4-(5-(2-(N-(tert- butyl)sulfamoyl)-4-(1-(2,2,2- trifluoroethy1)-1H-pyrazol-4- yl)phenyl)thiazol-2- yl)cyclohexyl)carbamate |
| 5 | 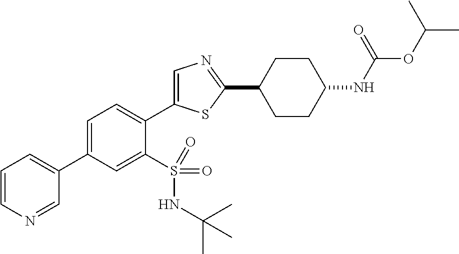 | isopropyl (trans-4-(5-(2-(N-(tert- butyl)sulfamoyl)-4-(pyridin-3- yl)phenyl)thiazol-2- yl)cyclohexyl)carbamate |
| 6 | 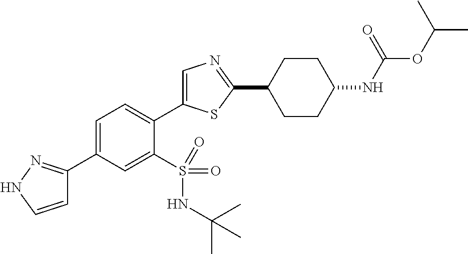 | isopropyl (trans-4-(5-(2-(N-(tert- butyl)sulfamoyl)-4-(1H-pyrazol- 3-yl)phenyl)thiazol-2- yl)cyclohexyl)carbamate |
| 7 | 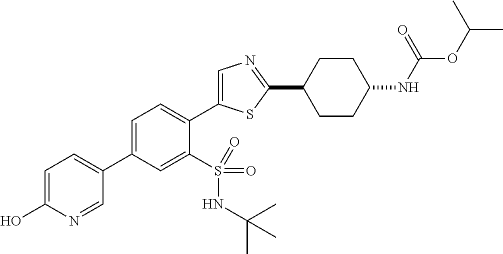 | isopropyl (trans-4-(5-(2-(N-(tert- butyl)sulfamoyl)-4-(6-hydroxy- pyridin-3-yl)phenyl)thiazol-2- yl)cyclohexyl)carbamate |
| 8 | 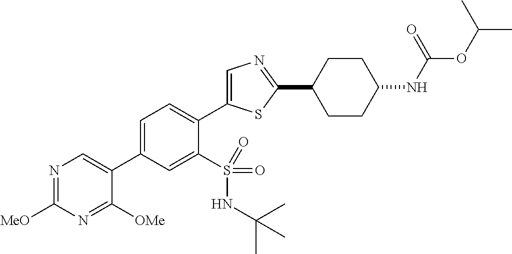 | isopropyl (trans-4-(5-(2-(N-(tert- butyl)sulfamoyl)-4-(2,4- dimethoxy pyrimidin-5- yl)phenyl)thiazol-2- yl)cyclohexyl)carbamate |
| 9 | 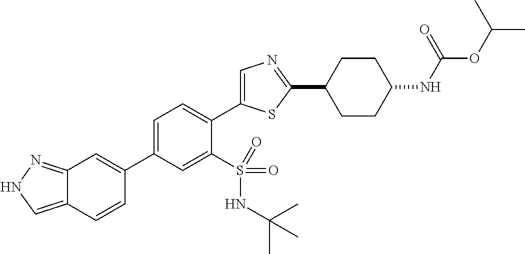 | isopropyl (trans-4-(5-(2-(N-(tert- butyl)sulfamoyl)-4-(2H-indazol- 6-yl)phenyl)thiazol-2- yl)cyclohexyl)carbamate |
| 10 | 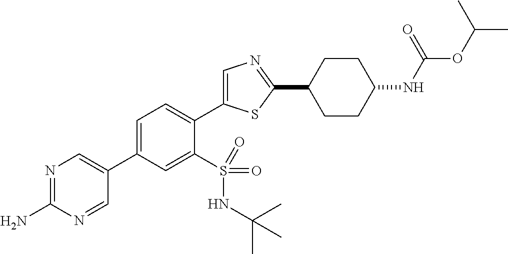 | isopropyl (trans-4-(5-(4-(2- aminopyrimidin-5-yl)-2-(N-(tert- butyl)sulfamoyl)phenyl)thiazol- 2-yl)cyclohexyl)carbamate |
| 11 | 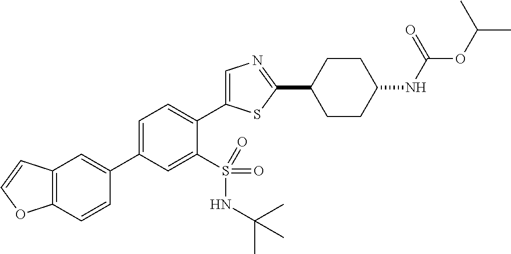 | isopropyl (trans-4-(5-(4- (benzofuran-5-yl)-2-(N-(tert- butyl)sulfamoyl)phenyl)thiazol- 2-yl)cyclohexyl)carbamate |
| 12 | 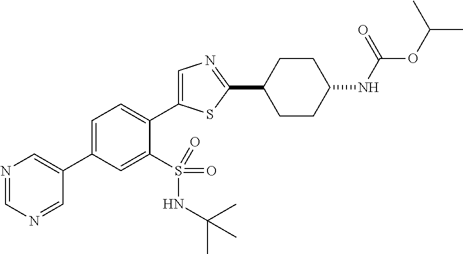 | isopropyl (trans-4-(5-(2-(N-(tert- butyl)sulfamoyl)-4-(pyrimidin-5- yl)phenyl)thiazol-2- yl)cyclohexyl)carbamate |
| 13 | 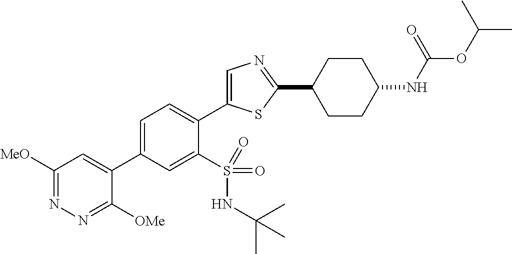 | isopropyl (trans-4-(5-(2-(N-(tert- butyl)sulfamoyl)-4-(3,6- dimethoxypyridazin-4- yl)phenyl)thiazol-2- yl)cyclohexyl)carbamate |
| 14 | 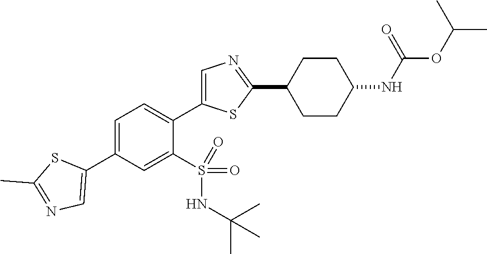 | isopropyl (trans-4-(5-(2-(N-(tert- butyl)sulfamoyl)-4-(2- methylthiazol-5- yl)phenyl)thiazol-2- yl)cyclohexyl)carbamate |
| 15 | 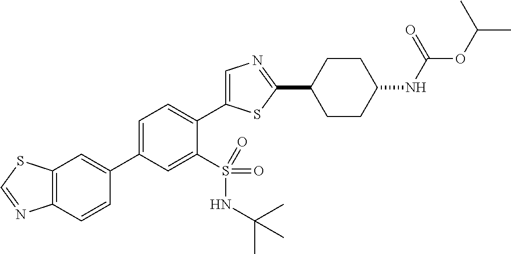 | isopropyl (trans-4-(5-(4- (benzo[d]thiazol-6-yl)-2-(N-(tert- butyl)sulfamoyl)phenyl)thiazol-2- yl)cyclohexyl)carbamate |
| 16 | 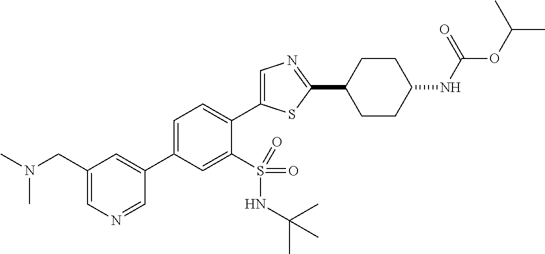 | isopropyl (trans-4-(5-(2-(N-(tert- butyl)sulfamoyl)-4-(5- ((dimethylamino)methyl)pyridin- 3-yl)phenyl)thiazol-2- yl)cyclohexyl)carbamate |
| 17 | 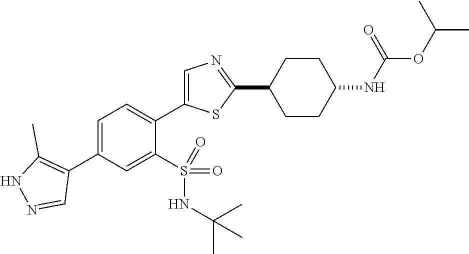 | isopropyl (trans-4-(5-(2-(N-(tert- butyl)sulfamoyl)-4-(5-methyl-1H- pyrazol-4-yl)phenyl)thiazol-2- yl)cyclohexyl)carbamate |
| 18 | 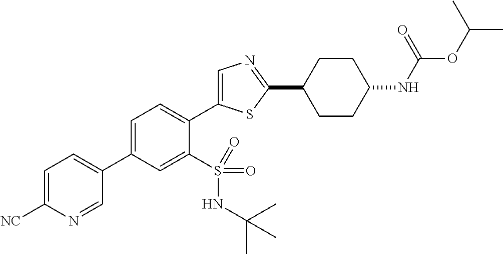 | isopropyl (trans-4-(5-(2-(N-(tert- butyl)sulfamoyl)-4-(6-cyano- pyridin-3-yl)phenyl)thiazol-2- yl)cyclohexyl)carbamate |
| 19 | 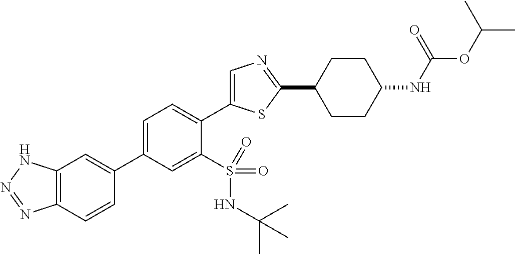 | isopropyl (trans-4-(5-(4-(1H- benzo[d][1,2,3 ]triazol-6-yl)-2-(N- (tert- butyl)sulfamoyl)phenyl)thiazol-2- yl)cyclohexyl)carbamate |
| 20 | 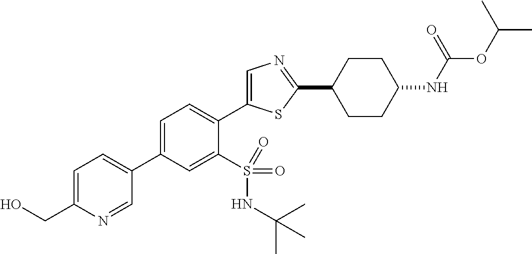 | isopropyl (trans-4-(5-(2-(N-(tert- butyl)sulfamoyl)-4-(6- (hydroxymethyl)pyridin-3- yl)phenyl)thiazol-2- yl)cyclohexyl)carbamate |
| 21 | 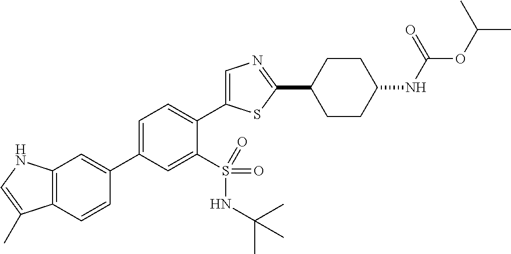 | isopropyl (trans-4-(5-(2-(N-(tert- butyl)sulfamoyl)-4-(3-methyl-1H- indol-6-yl)phenyl)thiazol-2- yl)cyclohexyl)carbamate |
| 22 | 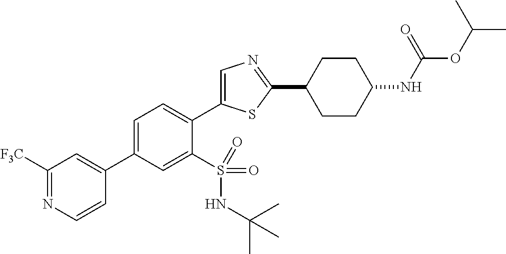 | isopropyl (trans-4-(5-(2-(N-(tert- butyl)sulfamoyl)-4-(2- (trifluoromethyl)pyridin-4- yl)phenyl)thiazol-2- yl)cyclohexyl)carbamate |
| 23 | 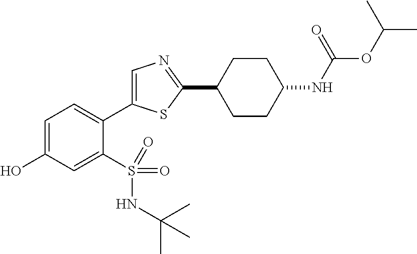 | isopropyl (trans-4-(5-(2-(N-(tert- butyl)sulfamoyl)-4-hydroxy- phenyl)thiazol-2- yl)cyclohexyl)carbamate |
| 24 | 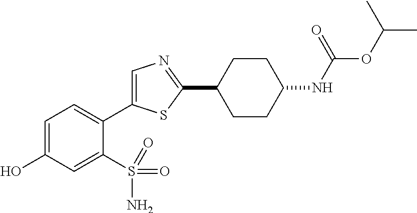 | isopropyl (trans-4-(5-(4-hydroxy- 2-sulfamoylphenyl)thiazol-2- yl)cyclohexyl)carbamate |
| 25 | 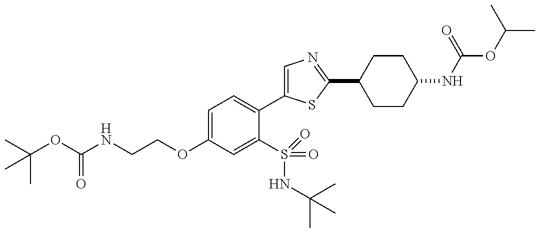 | isopropyl N-[trans-4-[5-[4-[2- (tert- butoxycarbonylamino)ethoxy]-2- (tert- butylsulfamoyl)phenyl]thiazol-2- yl]cyclohexyl]carbamate |
| 26 | 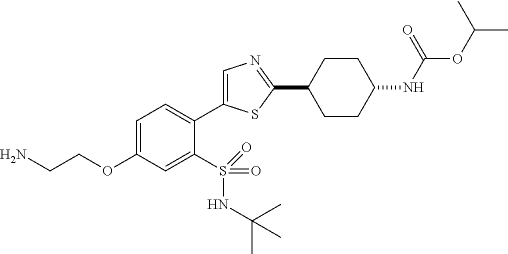 | isopropyl (trans-4-(5-(4-(2- aminoethoxy)-2-(N-(tert- butyl)sulfamoyl)phenyl)thiazol-2- yl)cyclohexyl)carbamate |
| 27 | 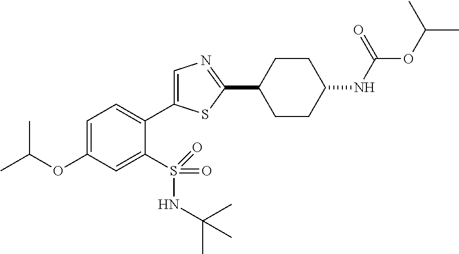 | isopropyl (trans-4-(5-(2-(N-(tert- butyl)sulfamoyl)-4-isopropoxy- phenyl)thiazol-2- yl)cyclohexyl)carbamate |
| 28 | 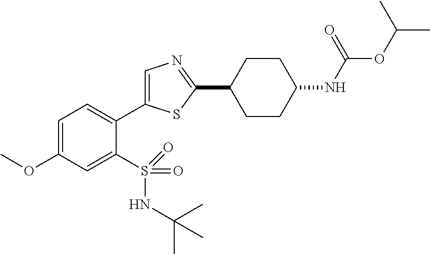 | isopropyl (trans-4-(5-(2-(N-(tert- butyl)sulfamoyl)-4-methoxy- phenyl)thiazol-2- yl)cyclohexyl)carbamate |
| 29 | 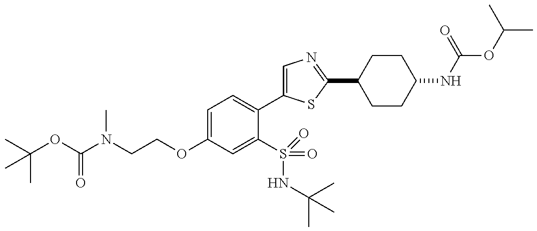 | tert-butyl (2-(3-(N-(tert- butyl)sulfamoyl)-4-(2-(trans-4- ((iso- propoxycarbonyl)amino)cyclohex- yl)thiazol-5- yl)phenoxy)ethyl)(methyl) carbamate |
| 30 | 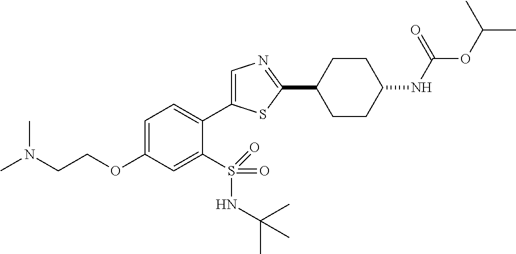 | isopropyl (trans-4-(5-(2-(N-(tert- butyl)sulfamoyl)-4-(2-(dimethyl- amino)ethoxy)phenyl)thiazol-2- yl)cyclohexyl)carbamate |
| 31 | 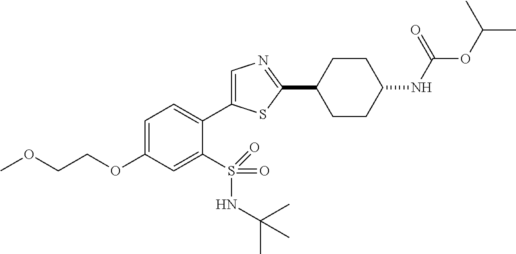 | isopropyl (trans-4-(5-(2-(N-(tert- butyl)sulfamoyl)-4-(2-methoxy- ethoxy)phenyl)thiazol-2- yl)cyclohexyl)carbamate |
| 32 | 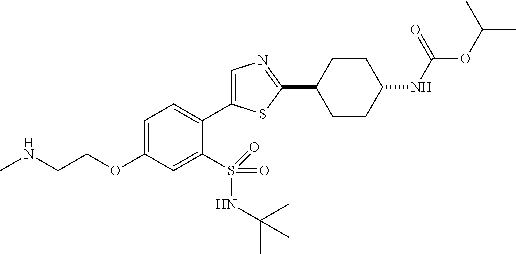 | isopropyl (trans-4-(5-(2-(N-(tert- butyl)sulfamoyl)-4-(2-(methyl- amino)ethoxy)phenyl)thiazol-2- yl)cyclohexyl)carbamate |
| 33 | 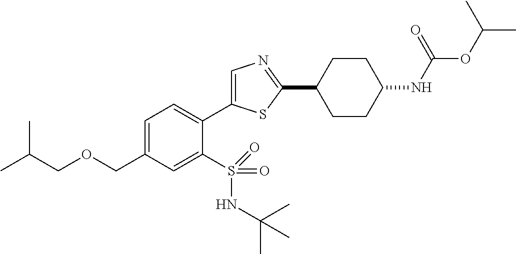 | isopropyl (trans-4-(5-(2-(N-(tert- butyl)sulfamoyl)-4- (isobutoxymethyl)phenyl)thiazol- 2-yl)cyclohexyl)carbamate |
| 34 | 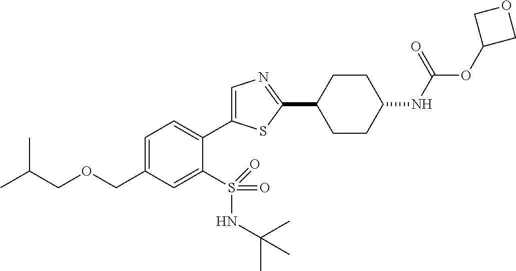 | oxetan-3-yl (trans-4-(5-(2-(N- (tert-butyl)sulfamoyl)-4- (isobutoxymethyl)phenyl)thiazol- 2-yl)cyclohexyl)carbamate |
| 35 | 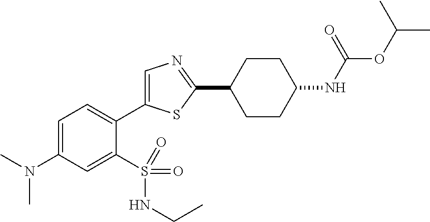 | isopropyl (trans-4-(5-(4- (dimethylamino)-2-(N- ethylsulfamoyl)phenyl)thiazol-2- yl)cyclohexyl)carbamate |
| 36 | 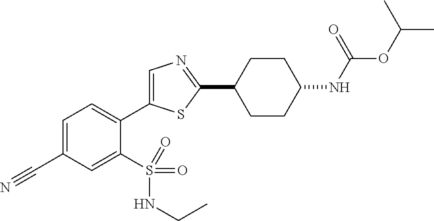 | isopropyl (trans-4-(5-(4-cyano-2- (N-ethylsulfamoyl)phenyl)thiazol- 2-yl)cyclohexyl)carbamate |
| 37 | 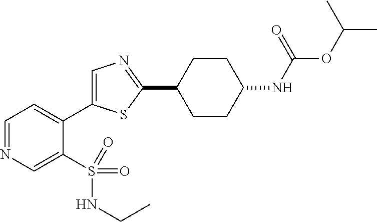 | isopropyl (trans-4-(5-(3-(N- ethylsulfamoyl)pyridin-4- yl)thiazol-2- yl)cyclohexyl)carbamate |
| 38 | 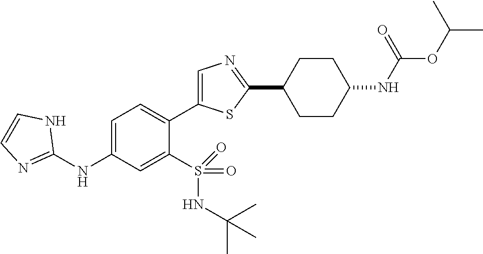 | isopropyl (trans-4-(5-(4-((1H- imidazol-2-yl)amino)-2-(N-(tert- butyl)sulfamoyl)phenyl)thiazol-2- yl)cyclohexyl)carbamate |
| 39 | 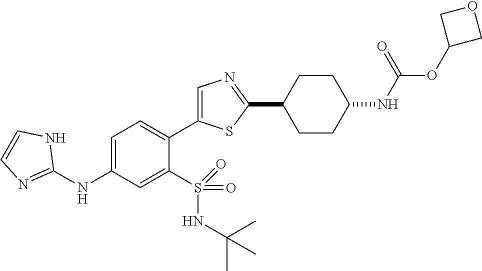 | oxetan-3-yl (trans-4-(5-(4-((1H- imidazol-2-yl)amino)-2-(N-(tert- butyl)sulfamoyl)phenyl)thiazol-2- yl)cyclohexyl)carbamate |
| 40 | 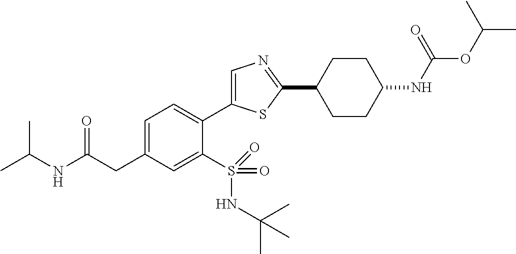 | isopropyl (trans-4-(5-(2-(N-(tert- butyl)sulfamoyl)-4-(2- (isopropylamino)-2- oxoethyl)phenyl)thiazol-2- yl)cyclohexyl)carbamate |
| 41 | 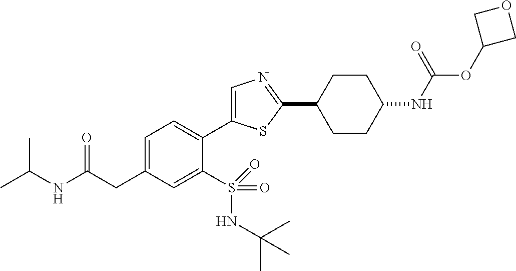 | oxetan-3-yl (trans-4-(5-(2-(N- (tert-butyl)sulfamoyl)-4-(2- (isopropylamino)-2- oxoethyl)phenyl)thiazol-2- yl)cyclohexyl)carbamate |
| 42 | 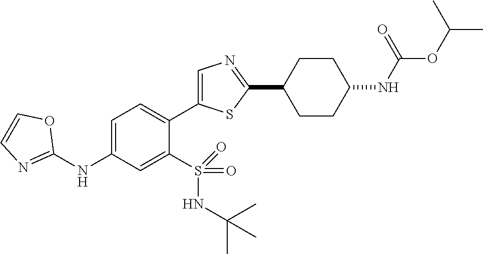 | isopropyl (trans-4-(5-(2-(N-(tert- butyl)sulfamoyl)-4-(oxazol-2- ylamino)phenyl)thiazol-2- yl)cyclohexyl)carbamate |
| 43 | 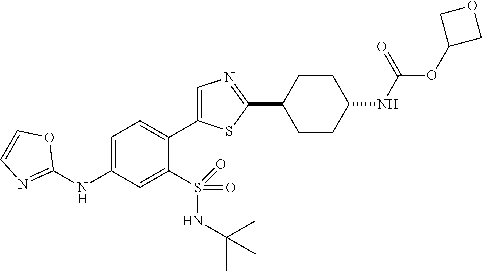 | oxetan-3-yl (trans-4-(5-(2-(N- (tert-butyl)sulfamoyl)-4-(oxazol- 2-ylamino)phenyl)thiazol-2- yl)cyclohexyl)carbamate |
| 44 | 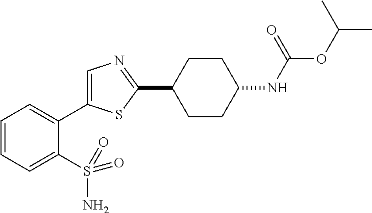 | isopropyl (trans-4-(5-(2- sulfamoylphenyl)thiazol-2- yl)cyclohexyl)carbamate |
| 45 | 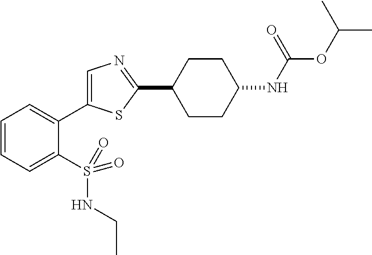 | isopropyl (trans-4-(5-(2-(N- ethylsulfamoyl)phenyl)thiazol-2- yl)cyclohexyl)carbamate |
| 46 | 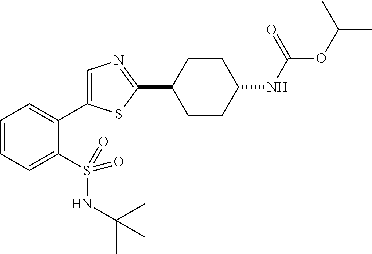 | isopropyl (trans-4-(5-(2-(N-(tert- butyl)sulfamoyl)phenyl)thiazol-2- yl)cyclohexyl)carbamate |
| 47 | 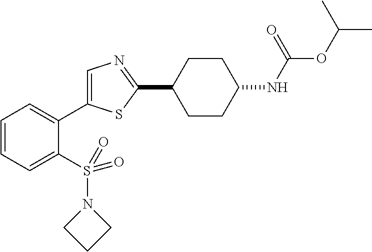 | isopropyl (trans-4-(5-(2-(azetidin- 1-ylsulfonyl)phenyl)thiazol-2- yl)cyclohexyl)carbamate |
| 48 | 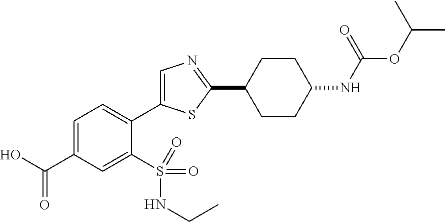 | 3-(N-ethylsulfamoyl)-4-(2-(trans- 4-((isopropoxy- carbonyl)amino)cyclo- hexyl)thiazol-5-yl)benzoic acid |
| 49 | 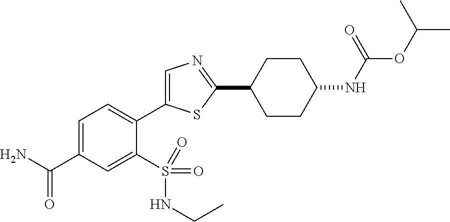 | isopropyl (trans-4-(5-(4- carbamoyl-2-(N- ethylsulfamoyl)phenyl)thiazol-2- yl)cyclohexyl)carbamate |
| 50 | 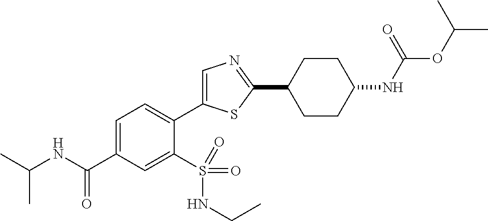 | isopropyl (trans-4-(5-(2-(N- ethylsulfamoyl)-4-(iso- propylcarbamoyl)phenyl)thiazol- 2-y1)cyclohexyl)carbamate |
| 51 | 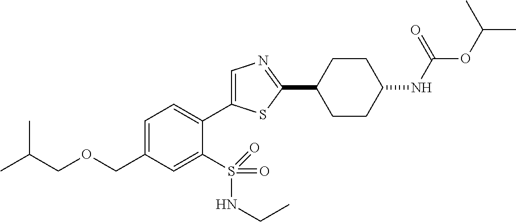 | isopropyl (trans-4-(5-(2-(N- ethylsulfamoyl)-4- (isobutoxymethyl)phenyl)thiazol- 2-yl)cyclohexyl)carbamate |
| 52 | 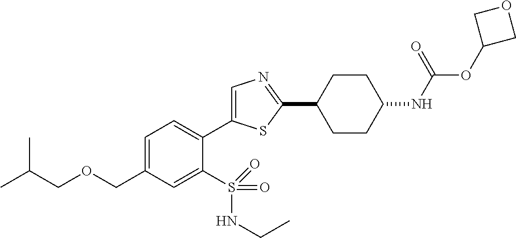 | oxetan-3-yl (trans-4-(5-(2-(N- ethylsulfamoyl)-4- (isobutoxymethyl)phenyl)thiazol- 2-yl)cyclohexyl)carbamate |
| 53 | 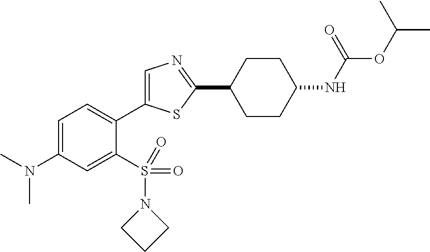 | isopropyl (trans-4-(5-(2-(azetidin- 1-ylsulfonyl)-4- (dimethylamino)phenyl)thiazol-2- yl)cyclohexyl)carbamate |
| 54 | 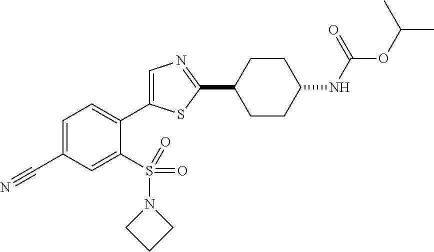 | isopropyl (trans-4-(5-(2-(azetidin- 1-ylsulfonyl)-4- cyanophenyl)thiazol-2- yl)cyclohexyl)carbamate |
| 55 | 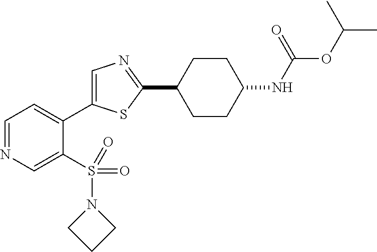 | isopropyl (trans-4-(5-(3-(azetidin- 1-ylsulfonyl)pyridin-4-yl)thiazol- 2-yl)cyclohexyl)carbamate |
| 56 | 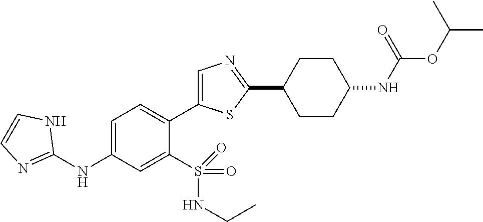 | isopropyl (trans-4-(5-(4-((1H- imidazol-2-yl)amino)-2-(N- ethylsulfamoyl)phenyl)thiazol-2- yl)cyclohexyl)carbamate |
| 57 | 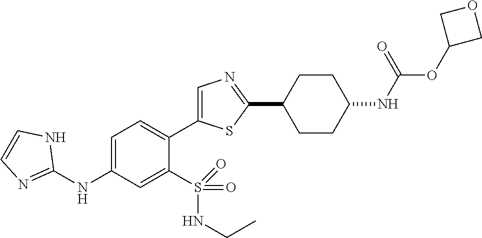 | oxetan-3-yl (trans-4-(5-(4-((1H- imidazol-2-yl)amino)-2-(N- ethylsulfamoyl)phenyl)thiazol-2- yl)cyclohexyl)carbamate |
| 58 | 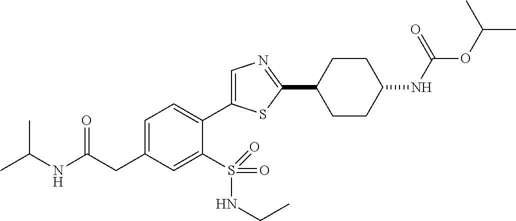 | isopropyl (trans-4-(5-(2-(N- ethylsulfamoyl)-4-(2- (isopropylamino)-2- oxoethyl)phenyl)thiazol-2- yl)cyclohexyl)carbamate |
| 59 | 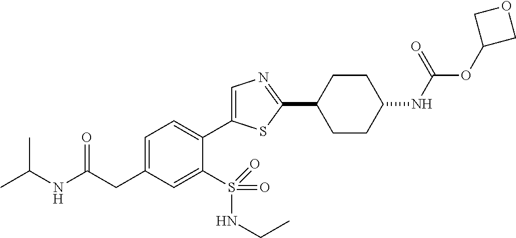 | oxetan-3-yl (trans-4-(5-(2-(N- ethylsulfamoy1)-4-(2- (isopropylamino)-2- oxoethyl)phenyl)thiazol-2- yl)cyclohexyl)carbamate |
| 60 | 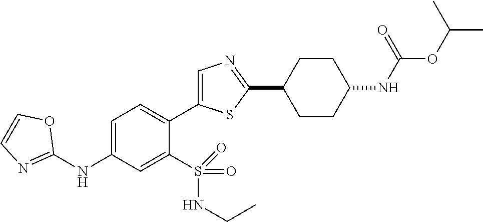 | isopropyl (trans-4-(5-(2-(N- ethylsulfamoyl)-4-(oxazol-2- ylamino)phenyl)thiazol-2- yl)cyclohexyl)carbamate |
| 61 | 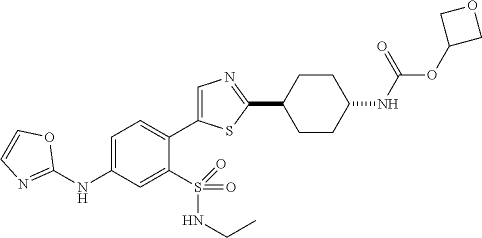 | oxetan-3-yl (trans-4-(5-(2-(N- ethylsulfamoyl)-4-(oxazol-2- ylamino)phenyl)thiazol-2- yl)cyclohexyl)carbamate |
| 62 | 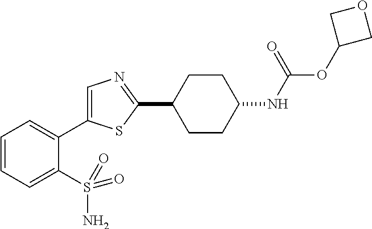 | oxetan-3-yl (trans-4-(5-(2- sulfamoylphenyl)thiazol-2- yl)cyclohexyl)carbamate |
| 63 | 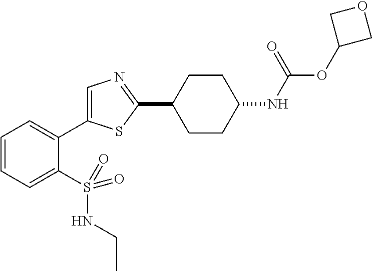 | oxetan-3-yl (trans-4-(5-(2-(N- ethylsulfamoyl)phenyl)thiazol-2- yl)cyclohexyl)carbamate |
| 64 | 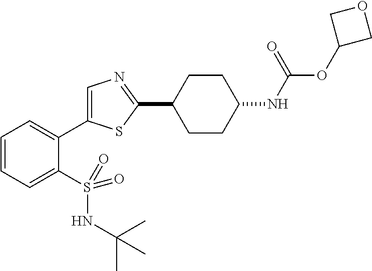 | oxetan-3-yl (trans-4-(5-(2-(N- (tert- butyl)sulfamoyl)phenyl)thiazol-2- yl)cyclohexyl)carbamate |
| 65 | 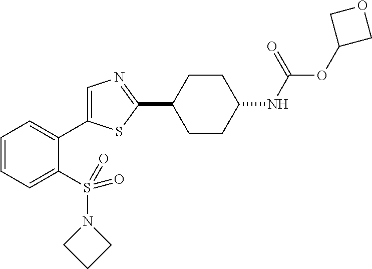 | oxetan-3-yl (trans-4-(5-(2- (azetidin-1- ylsulfonyl)phenyl)thiazol-2- yl)cyclohexyl)carbamate |
| 66 | 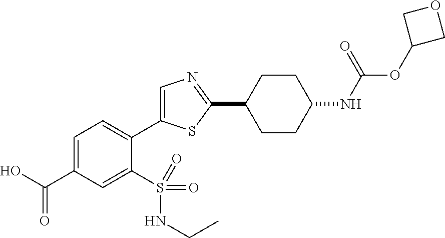 | 3-(N-ethylsulfamoyl)-4-(2-(trans- 4-(((oxetan-3-yl- oxy)carbonyl)amino)cyclohexyl) thiazol-5-yl)benzoic acid |
| 67 | 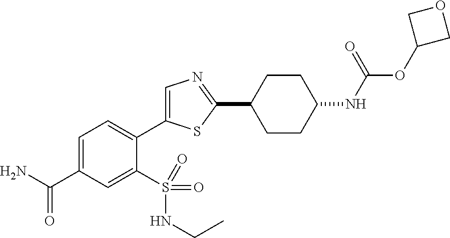 | oxetan-3-yl (trans-4-(5-(4- carbamoyl-2-(N- ethylsulfamoyl)phenyl)thiazol-2- yl)cyclohexyl)carbamate |
| 68 | 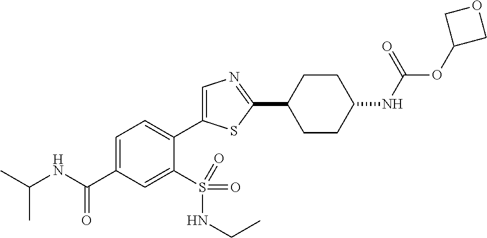 | oxetan-3-yl (trans-4-(5-(2-(N- ethylsulfamoyl)-4- (isopropylcarbamoyl)phenyl)thia- zol-2-yl)cyclohexyl)carbamate |
| 69 | 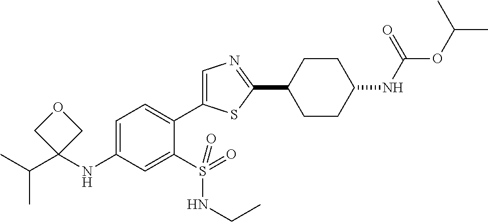 | isopropyl (trans-N-(4-(5-(2- (ethylsulfamoyl)-4-((3-isopropyl- oxetan-3- yl)amino)phenyl)thiazol-2- yl)cyclohexyl))carbamate |
| 70 | 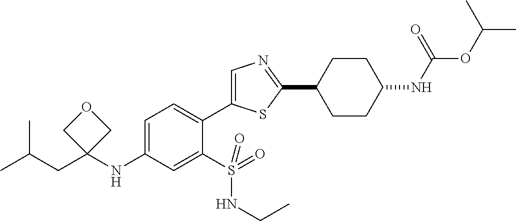 | isopropyl trans-N-[4-[5-[2- (ethylsulfamoyl)-4-[(3- isobutyloxetan-3- yl)amino]phenyl]thiazol-2- yl]cyclohexyl]carbamate |
| 71 | 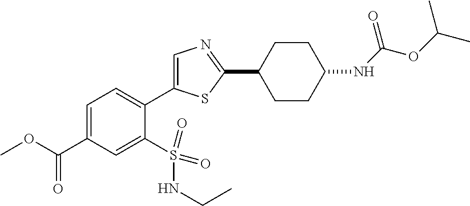 | methyl trans- 3- (ethylsulfamoyl)-4-[2-[4- (isopropoxycarbonyl- amino)cyclohexyl]thiazol-5- yl]benzoate |
| 72 | 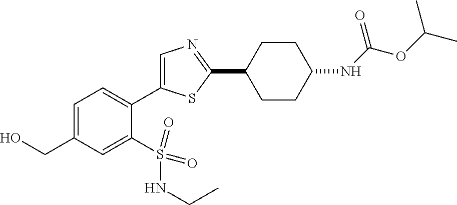 | isopropyl trans-N-[4-[5-[2- (ethylsulfamoyl)-4-(hydroxy- methyl)phenyl]thiazol-2- yl]cyclohexyl]carbamate |
| 73 | 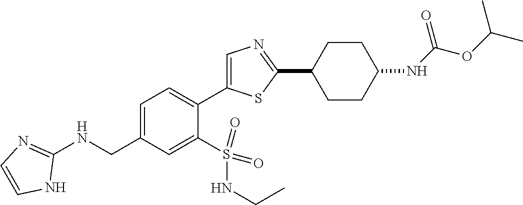 | isopropyl trans-N-[4-[5-[2- (ethylsulfamoy1)-4-[(1H- imidazol-2- ylamino)methyl]phenyl]thiazol- 2-yl]cyclohexyl]carbamate |
| 74 | 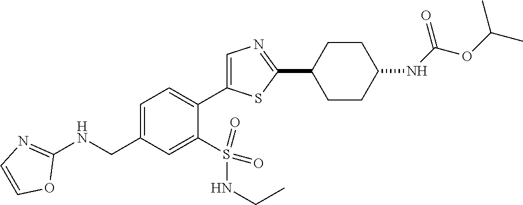 | isopropyl trans-N-[4-[5-[2- (ethylsulfamoyl)-4-[(oxazol-2- ylamino) methyl]phenyl]thiazol- 2-yl]cyclohexyl]carbamate |
| 75 | 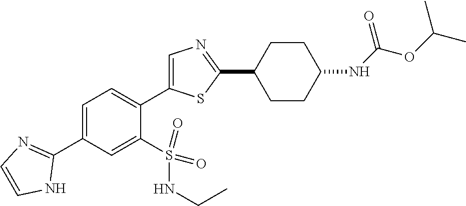 | isopropyl trans-N-[4-[5-[2- (ethylsulfamoyl)-4-(1H- imidazol-2-yl)phenyl]thiazol-2- yl]cyclohexyl]carbamate |
| 76 | 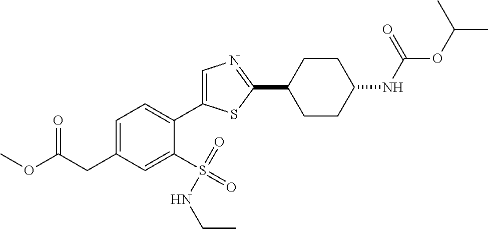 | methyl trans-2-[3- (ethylsulfamoyl)-4-[2-[4- (isopropoxycarbonylamino) cyclohexyl]thiazol-5- yl]phenyl]acetate |
| 77 | 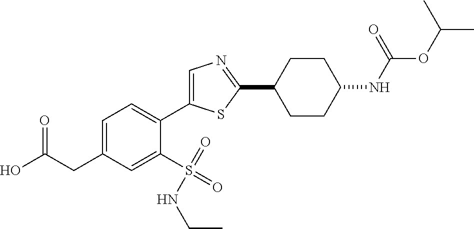 | trans-2-[3-(ethylsulfamoyl)-4-[2- [4-(isopropoxycarbonyl- amino)cyclohexyl]thiazol-5- yl]phenyl]acetic acid |
| 78 | 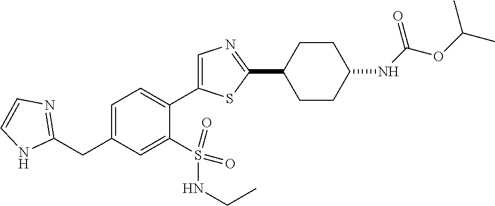 | isopropyl trans-N-[4-[5-[2- (ethylsulfamoyl)-4-(1H- imidazol-2- ylmethyl)phenyl]thiazol-2- yl]cyclohexyl]carbamate |
| 79 | 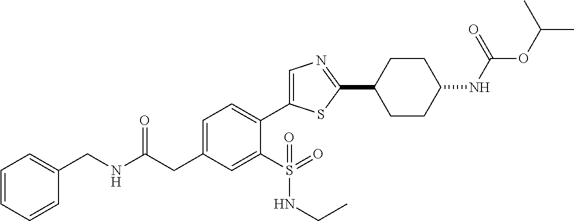 | isopropyl trans-N-[4-[5-[4-[2- (benzylamino)-2-oxo-ethyl]-2- (ethyl-sulfamoyl)phenyl]thiazol- 2-yl]cyclohexyl]carbamate |
| 80 | 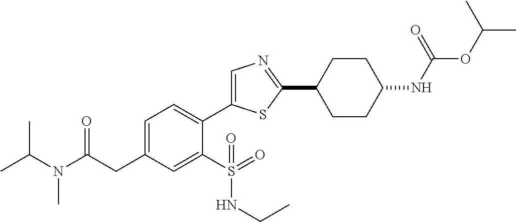 | isopropyl trans-N-[4-[5-[4-[2- (isopropyl(methyl)amino)-2-oxo- ethyl]-2-(ethyl- sulfamoyl)phenyl]thiazol-2- yl]cyclohexyl]carbamate |
| 81 | 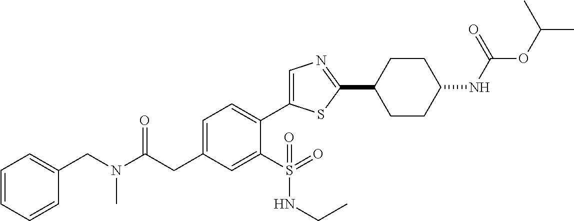 | isopropyl trans-N-[4-[5-[4-[2- (benzyl(methyl)amino)-2-oxo- ethyl]-2-(ethyl- sulfamoyl)phenyl]thiazol-2- yl]cyclohexyl]carbamate |
| 82 | 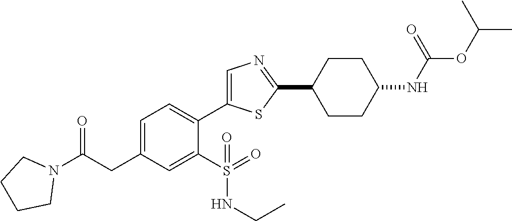 | isopropyl trans-N-[4-[5-[4-[2- oxo-2-(pyrrolidin-1-yl)ethyl]-2- (ethyl-sulfamoyl)phenyl]thiazol- 2-yl]cyclohexyl]carbamate |
| 83 | 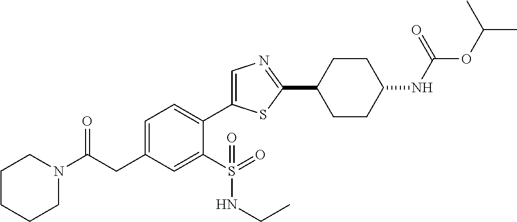 | isopropyl trans-N-[4-[5-[4-[2- oxo-2-(piperidin-1-yl)ethyl]-2- (ethyl-sulfamoyl)phenyl]thiazol- 2-yl]cyclohexyl]carbamate |
| 84 | 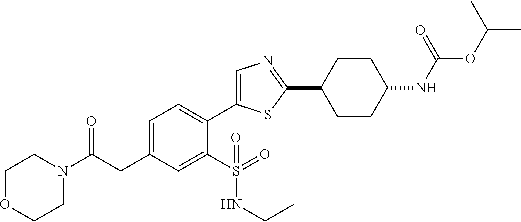 | isopropyl trans-N-[4-[5-[4-[2- oxo-2-(morpholin-4-yl)ethyl]-2- (ethyl-sulfamoyl)phenyl]thiazol- 2-yl]cyclohexyl]carbamate |
| 85 | 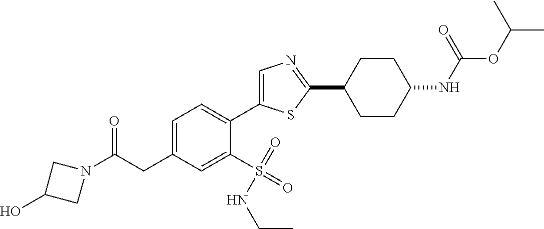 | isopropyl trans-N-[4-[5-[4-[2- oxo-2-(3-hydroxyazetidin-1- yl)ethyl]-2-(ethyl- sulfamoyl)phenyl]thiazol-2- yl]cyclohexyl]carbamate |
| 86 | 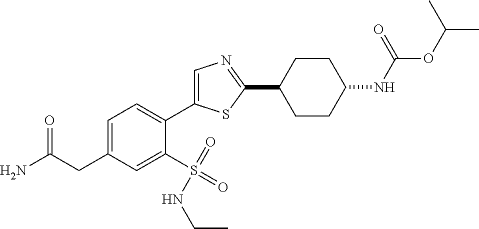 | isopropyl trans-N-[4-[5-[4-[2- amino-2-oxo-ethyl]-2-(ethyl- sulfamoyl)phenyl]thiazol-2- yl]cyclohexyl]carbamate |
| 87 | 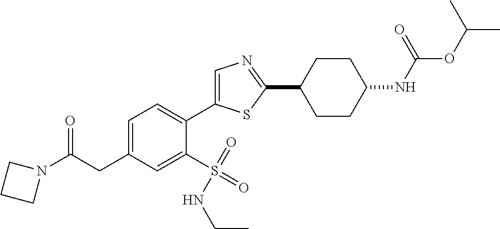 | isopropyl trans-N-[4-[5-[4-[2- oxo-2-(azetidin-1-yl)ethyl]-2- (ethyl-sulfamoyl)phenyl]thiazol- 2-yl]cyclohexyl]carbamate |
| 88 | 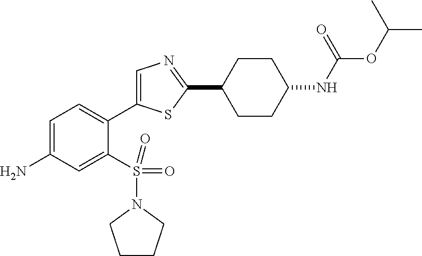 | isopropyl trans-N-[4-[5-(4- amino-2-pyrrolidin-1-ylsulfonyl- phenyl)thiazol-2- yl]cyclohexyl]carbamate |
| 89 | 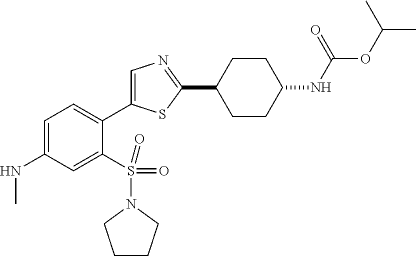 | isopropyl trans-N-[4-[5-(4- (methylamino)-2-pyrrolidin-1- ylsulfonyl-phenyl)thiazol-2- yl]cyclohexyl]carbamate |
| 90 | 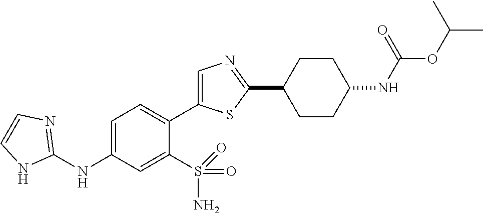 | isopropyl trans-N-[4-[5-[4-(1H- imidazol-2-ylamino)-2- sulfamoyl-phenyl]thiazol-2- yl]cyclohexyl]carbamate |
| 91 | 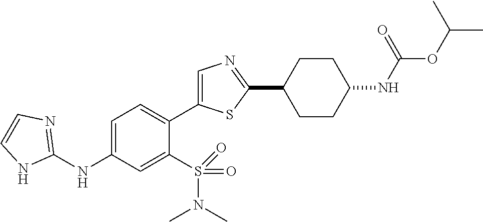 | isopropyl trans-N-[4-[5-[2- (dimethylsulfamoyl)-4-(1H- imidazol-2- ylamino)phenyl]thiazol-2- yl]cyclohexyl]carbamate |
| 92 | 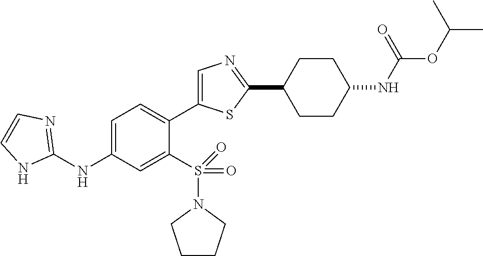 | isopropyl trans-N-[4-[5-[4-(1H- imidazol-2-ylamino)-2- pyrrolidin-1-ylsulfonyl- phenyl]thiazol-2- yl]cyclohexyl]carbamate |
| 93 | 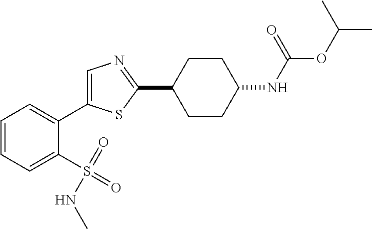 | isopropyl trans-N-[4-[5-(2- methylsulfamoylphenyl)thiazol- 2-yl]cyclohexyl]carbamate |
| 94 | 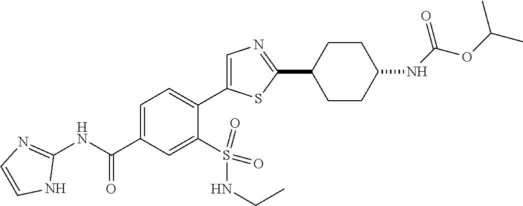 | isopropyl trans-N-[4-[5-[2- (ethylsulfamoyl)-4-(1H- imidazol-2- ylcarbamoyl)phenyl]thiazol-2- yl]cyclohexyl]carbamate |
| 95 | 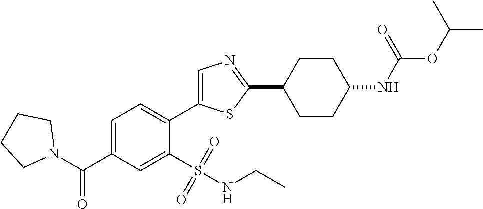 | isopropyl trans-N-[4-[5-[4- pyrrolidin-1-ylcarbonyl-2- (ethylsulfamoyl)phenyl]thiazol- 2-yl]cyclohexyl]carbamate |
| 96 | 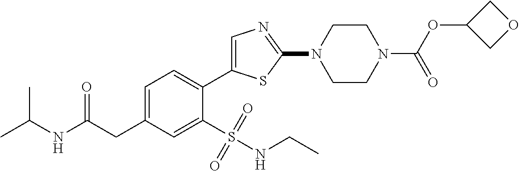 | oxetan-3-y1 4-(5-(2-(N- ethylsulfamoyl)-4-(2- (isopropylamino)-2- oxoethyl)phenyl)thiazol-2- yl)piperazine-1-carboxylate |
| 97 | 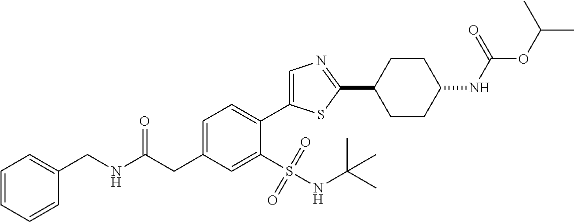 | isopropyl trans-N-[4-[5-[4-[2- (benzylamino)-2-oxo-ethyl]-2- (tert- butylsulfamoyl)phenyl]thiazol-2- yl]cyclohexyl]carbamate |
| 98 | 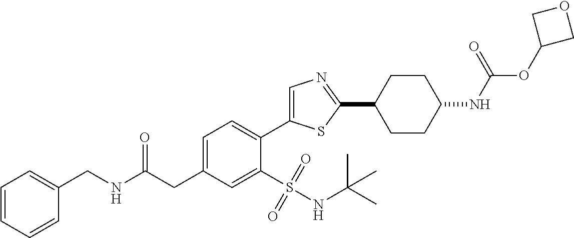 | oxetan-3-yl trans-N-[4-[5-[4-[2- (benzylamino)-2-oxo-ethyl]-2- (tert- butylsulfamoyl)phenyl]thiazol-2- yl]cyclohexyl]carbamate |
| 99 | 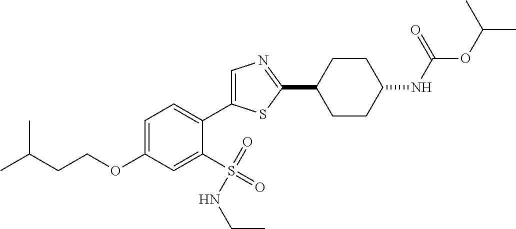 | isopropyl trans-N-[4-[5-[2- (ethylsulfamoyl)-4-isopentyloxy- phenyl]thiazol-2- yl]cyclohexyl]carbamate |
| 100 | 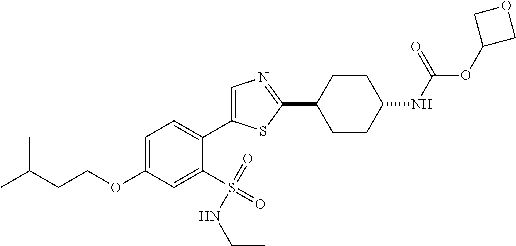 | oxetan-3-yl trans-N-[4-[5-[2- (ethylsulfamoyl)-4-isopentyloxy- phenyl]thiazol-2- yl]cyclohexyl]carbamate |
| 101 | 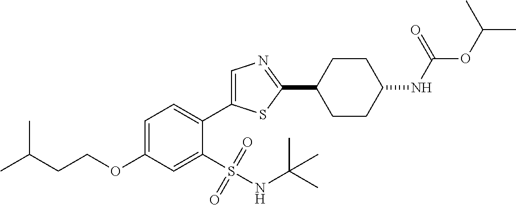 | isopropyl trans-N-[4-[5-[2-(tert- butylsulfamoyl)-4-isopentyloxy- phenyl]thiazol-2- yl]cyclohexyl]carbamate |
| 102 | 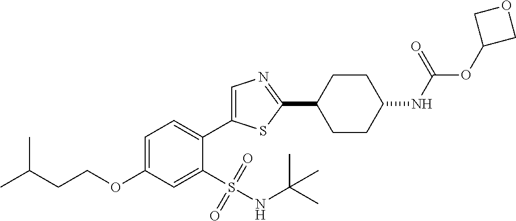 | oxetan-3-yl trans-N-[4-[5-[2- (tert-butylsulfamoyl)-4- isopentyloxy-phenyl]thiazol-2- yl]cyclohexyl]carbamate |
| 103 | 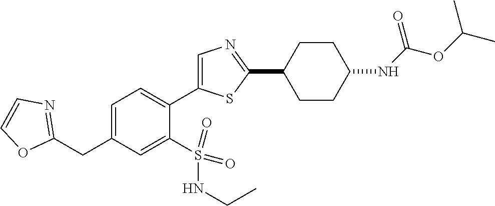 | isopropyl trans-N-[4-[5-[2- (ethylsulfamoyl)-4-(oxazol-2- ylmethyl)phenyl]thiazol-2- yl]cyclohexyl]carbamate |
| 104 | 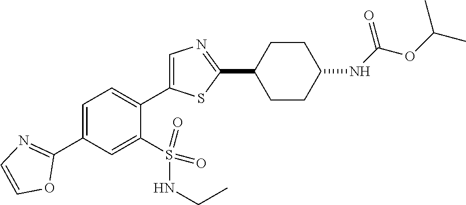 | isopropyl trans-N-[4-[5-[2- (ethylsulfamoyl)-4-oxazol-2-yl- phenyl]thiazol-2- yl]cyclohexyl]carbamate |
| 105 | 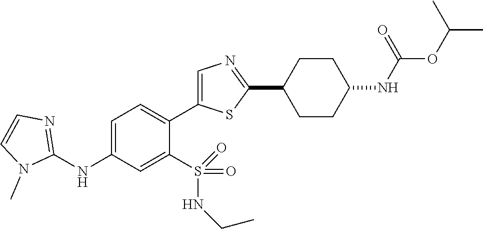 | isopropyl trans-N-[4-[5-[2- (ethylsulfamoyl)-4-[(1- methylimidazol-2- yl)amino]phenyl]thiazol-2- yl]cyclohexyl]carbamate |
| 106 | 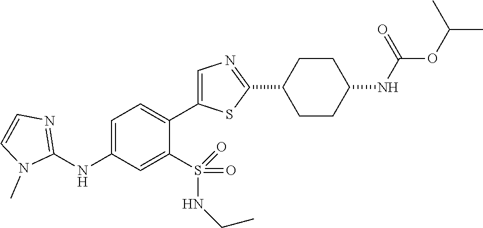 | isopropyl cis-N-[4-[5-[2- (ethylsulfamoyl)-4-[(1- methylimidazol-2- yl)amino]phenyl]thiazol-2- yl]cyclohexyl]carbamate |
| 107 | 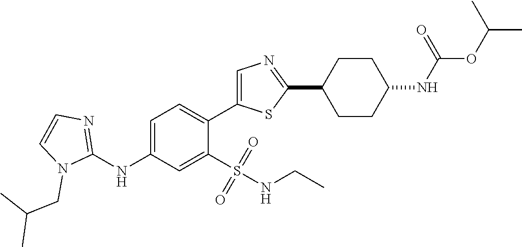 | isopropyl trans-N-[4-[5-[2- (ethylsulfamoyl)-4-[(1-isobutyl- imidazol-2- yl)amino]phenyl]thiazol-2- yl]cyclohexyl]carbamate |
| 108 | 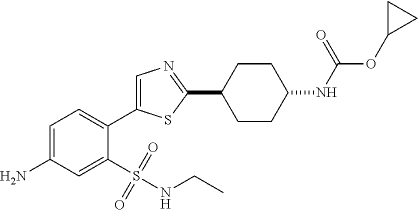 | cyclopropyl trans-N-[4-[5-[4- amino-2-(ethylsulfamoyl) phenyl]thiazol-2- yl]cyclohexyl]carbamate |
| 109 | 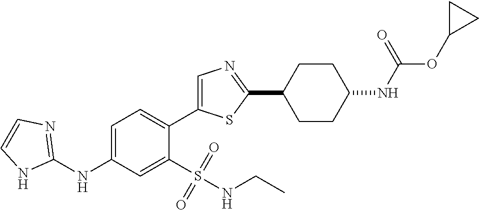 | cyclopropyl trans-N-[4-[5-[2- (ethylsulfamoyl)-4-(1H- imidazol-2- ylamino)phenyl]thiazol-2- yl]cyclohexyl]carbamate |
| 110 | 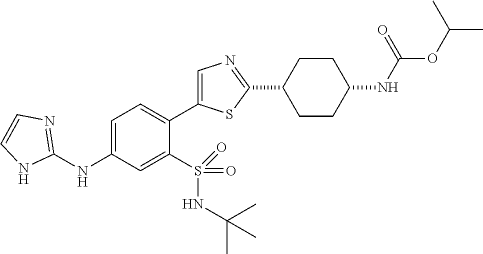 | isopropyl cis-N-[4-[5-[2-(tert- butylsulfamoyl)-4-(1H-imidazol- 2-ylamino)phenyl]thiazol-2- yl]cyclohexyl]carbamate |
| 111 | 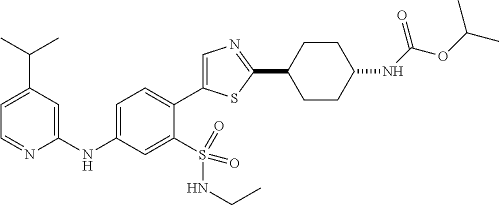 | isopropyl trans-N-[4-[5-[2- (ethylsulfamoy1)-4-[(4-isopropyl- 2-pyridyl)amino]phenyl]thiazol- 2-yl]cyclohexyl]carbamate |
| 112 | 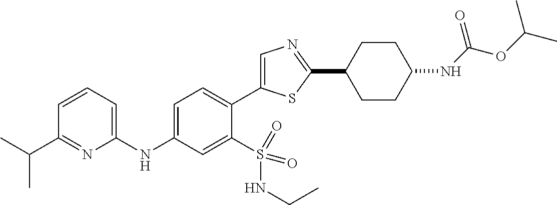 | isopropyl trans-N-[4-[5-[2- (ethylsulfamoyl)-4-[(6-isopropyl- 2-pyridyl)amino]phenyl]thiazol- 2-yl]cyclohexyl]carbamate |
| 113 | 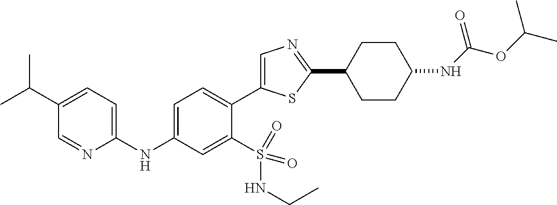 | isopropyl trans-N-[4-[5-[2- (ethylsulfamoyl)-4-[(5-isopropyl- 2-pyridyl)amino]phenyl]thiazol- 2-yl]cyclohexyl]carbamate |
| 114 | 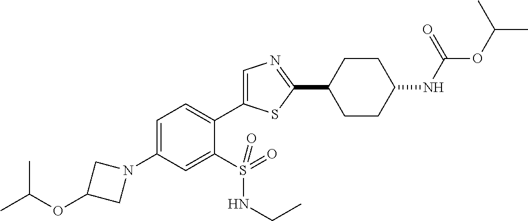 | isopropyl trans-N-[4-[5-[2- (ethylsulfamoyl)-4-[3- isopropoxyazetidin-1- yl]phenyl]thiazol-2- yl]cyclohexyl]carbamate |
| 115 | 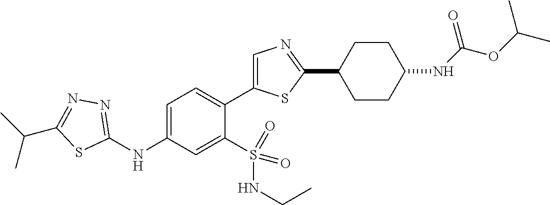 | isopropyl trans-N-[4-[5-[2- (ethylsulfamoyl)-4-[(5-isopropyl- 1,3,4-thiadiazol-2- y)amino]phenyl]thiazol-2- yl]cyclohexyl]carbamate |
| 116 | 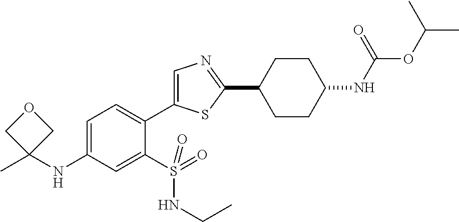 | isopropyl trans-N-[4-[5-[2- (ethylsulfamoyl)-4-[(3- methyloxetan-3- yl)amino]phenyl]thiazol-2- yl]cyclohexyl]carbamate |
| 117 | 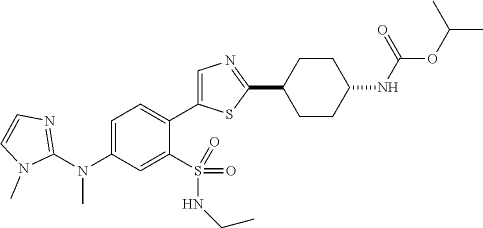 | isopropyl trans-N-[4-[5-[2- (ethylsulfamoyl)-4-[methyl(1- methy1-1H-imidazol-2- yl)amino]phenyl]thiazol-2- yl]cyclohexyl]carbamate |
| 118 | 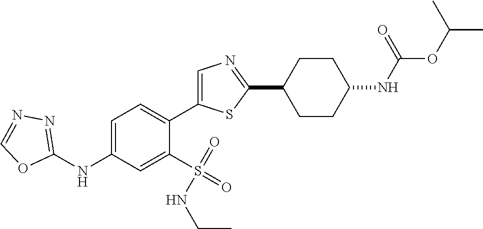 | isopropyl trans-N-[4-[5-[2- (ethylsulfamoyl)-4-[(1,3,4- oxadiazol-2- yl)amino]phenyl]thiazol-2- yl]cyclohexyl]carbamate |
| 119 | 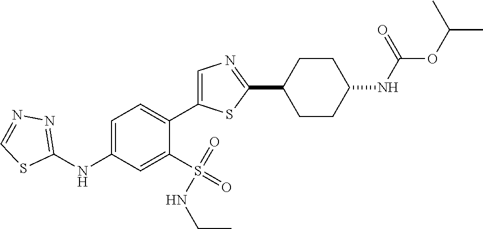 | isopropyl trans-N-[4-[5-[2- (ethylsulfamoyl)-4-[(1,3,4- thiadiazol-2- yl)amino]phenyl]thiazol-2- yl]cyclohexyl]carbamate |
| 120 | 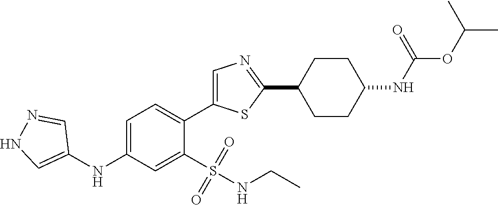 | isopropyl trans-N-[4-[5-[2- (ethylsulfamoyl)-4-[(1H-pyrazol- 4-yl)amino]phenyl]thiazol-2- yl]cyclohexyl]carbamate |
| 121 | 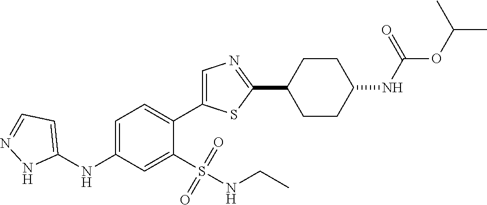 | isopropyl trans-N-[4-[5-[2- (ethylsulfamoyl)-4-[(1H-pyrazol- 5-yl)amino]phenyl]thiazol-2- yl]cyclohexyl]carbamate |
| 122 | 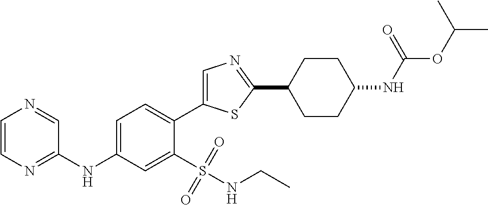 | isopropyl trans-N-[4-[5-[2- (ethylsulfamoyl)-4-[(pyrazin-2- yl)amino]phenyl]thiazol-2- yl]cyclohexyl]carbamate |
| 123 | 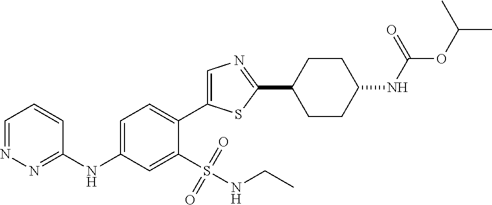 | isopropyl trans-N-[4-[5-[2- (ethylsulfamoyl)-4-[(pyridazin-3- yl)amino]phenyl]thiazol-2- yl]cyclohexyl]carbamate |
| 124 | 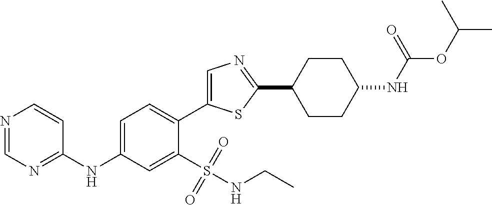 | isopropyl trans-N-[4-[5-[2- (ethylsulfamoyl)-4-[(pyrimidin- 4-yl)amino]phenyl]thiazol-2- yl]cyclohexyl]carbamate |
| 125 | 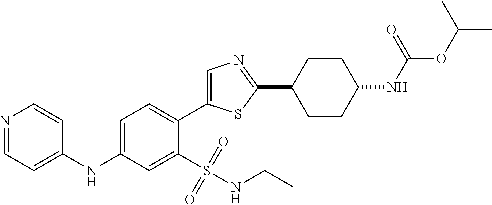 | isopropyl trans-N-[4-[5-[2- (ethylsulfamoyl)-4-[(pyridin-4- yl)amino]phenyl]thiazol-2- yl]cyclohexyl]carbamate |
| 126 | 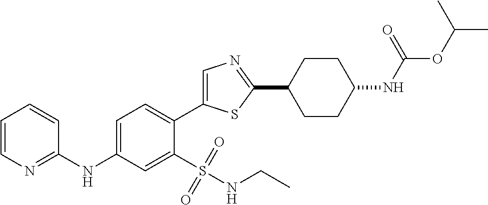 | isopropyl trans-N-[4-[5-[2- (ethylsulfamoyl)-4-[(pyridin-2- yl)amino]phenyl]thiazol-2- yl]cyclohexyl]carbamate |
| 127 | 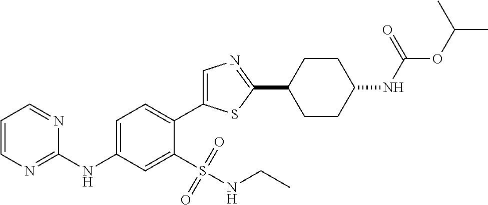 | isopropyl trans-N-[4-[5-[2- (ethylsulfamoyl)-4-[(pyrimidin- 2-yl)amino]phenyl]thiazol-2- yl]cyclohexyl]carbamate |
| 128 | 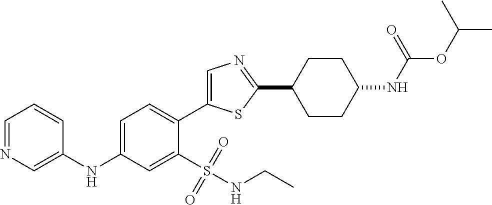 | isopropyl trans-N-[4-[5-[2- (ethylsulfamoy1)-4-[(pyridin-3- yl)amino]phenyl]thiazol-2- yl]cyclohexyl]carbamate |
| 129 | 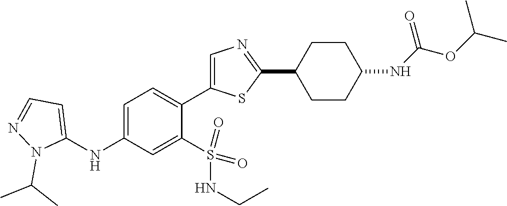 | isopropyl trans-N-[4-[5-[2- (ethylsulfamoyl)-4-[(1-isopropyl- 1H-pyrazol-5- yl)amino]phenyl]thiazol-2- yl]cyclohexyl]carbamate |
| 130 | 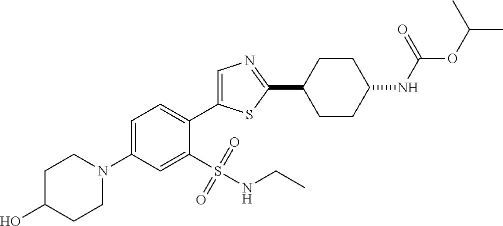 | isopropyl trans-N-[4-[5-[2- (ethylsulfamoyl)-4-[4- hydroxypiperidin-1- yl]phenyl]thiazol-2- yl]cyclohexyl]carbamate |
| 131 | 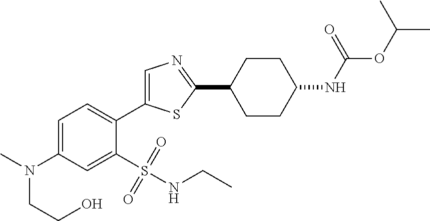 | isopropyl trans-N-[4-[5-[2- (ethylsulfamoy1)-4-[(2- hydroxyethyl) (methyl))amino]phenyl]thiazol- 2-yl]cyclohexyl]carbamate |
| 132 | 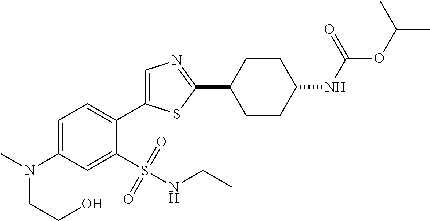 | isopropyl trans-N-[4-[5-[2- (ethylsulfamoyl)-4-[(2- methoxyethyl) (methyl))amino]phenyl]thiazol- 2-yl]cyclohexyl]carbamate |
| 133 | 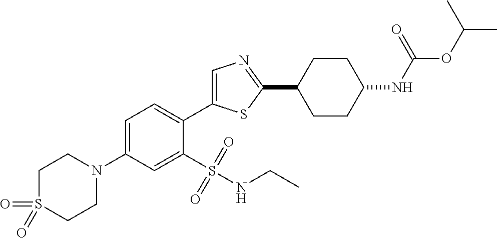 | isopropyl trans-N-[4-[5-[2- (ethylsulfamoyl)-4-[1,1-di- oxidothiomorpholino]phenyl]thi- azol-2-yl]cyclohexyl]carbamate |
| 134 | 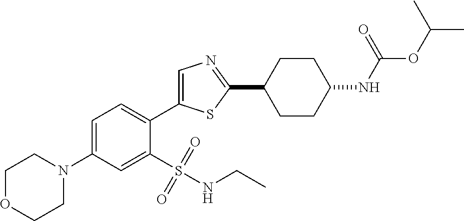 | isopropyl trans-N-[4-[5-[2- (ethylsulfamoyl)-4- [morpholino]phenyl]thiazol-2- yl]cyclohexyl]carbamate |
| 135 | 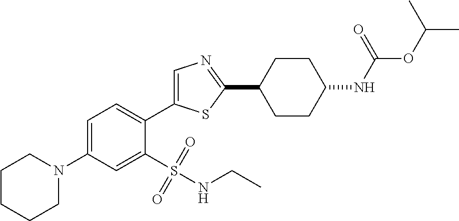 | isopropyl trans-N-[4-[5-[2- (ethylsulfamoyl)-4-[piperidin-1- yl]phenyl]thiazol-2- yl]cyclohexyl]carbamate |
| 136 | 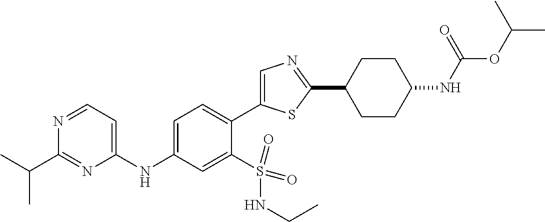 | isopropyl trans-N-[4-[5-[2- (ethylsulfamoyl)-4-[(2- isopropylpyrimidin-4- yl)amino]phenyl]thiazol-2- yl]cyclohexyl]carbamate |
| 137 | 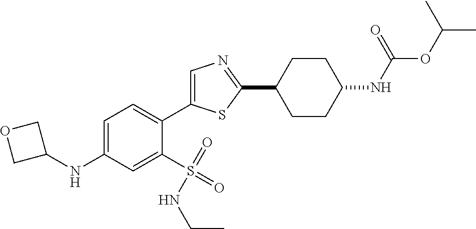 | isopropyl trans-N-[4-[5-[2- (ethylsulfamoyl)-4-[(oxetan-3- yl)amino]phenyl]thiazol-2- yl]cyclohexyl]carbamate |
| 138 | 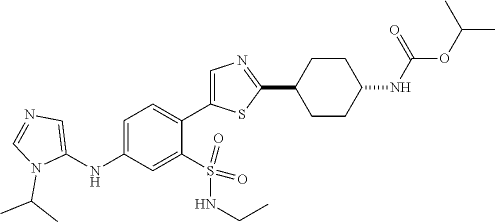 | isopropyl trans-N-[4-[5-[2- (ethylsulfamoyl)-4-[(3- isopropylimidazol-4- yl)amino]phenyl]thiazol-2- yl]cyclohexyl]carbamate |
| 139 | 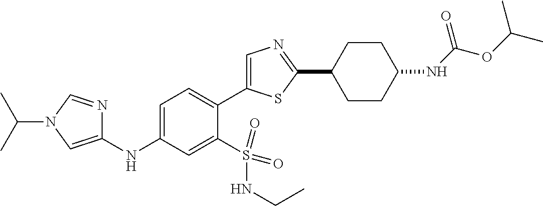 | isopropyl trans-N-[4-[5-[2- (ethylsulfamoyl)-4-[(1-isopropyl- 1H-imidazol-4- yl)amino]phenyl]thiazol-2- yl]cyclohexyl]carbamate |
| 140 | 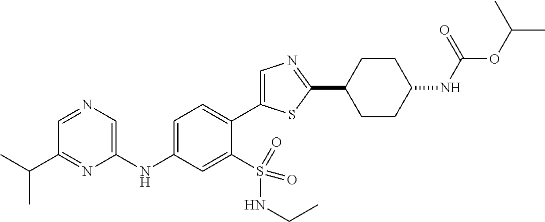 | isopropyl trans-N-[4-[5-[2- (ethylsulfamoyl)-4-[(6- isopropylpyrazin-2- yl)amino]phenyl]thiazol-2- yl]cyclohexyl]carbamate |
| 141 | 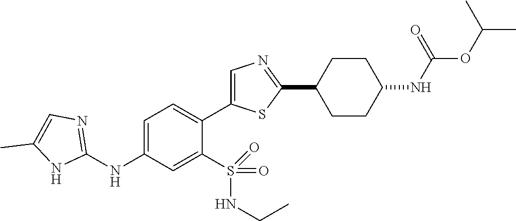 | isopropyl trans-N-[4-[5-[2- (ethylsulfamoyl)-4-[(5-methyl- 1H-imidazol-2- yl)amino]phenyl]thiazol-2- yl]cyclohexyl]carbamate |
| 142 | 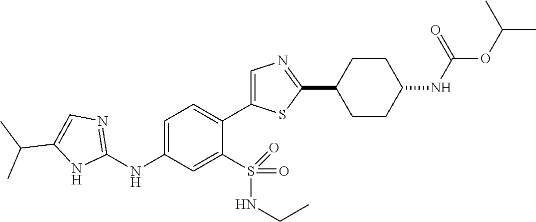 | isopropyl trans-N-[4-[5-[2- (ethylsulfamoyl)-4-[(5-isopropyl- 1H-imidazol-2- yl)amino]phenyl]thiazol-2- yl]cyclohexyl]carbamate |
| 143 | 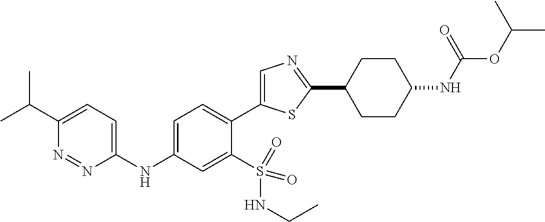 | isopropyl trans-N-[4-[5-[2- (ethylsulfamoyl)-4-[(6- isopropylpyridazin-3- yl)amino]phenyl]thiazol-2- yl]cyclohexyl]carbamate |
| 144 | 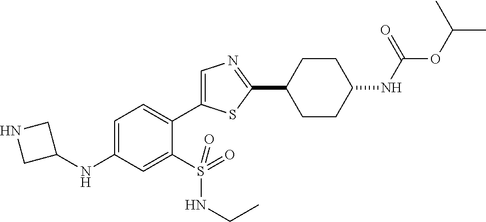 | isopropyl trans-N-[4-[5-[4- (azetidin-3-ylamino)-2-(ethyl- sulfamoyl)phenyl]thiazol-2- yl]cyclohexyl]carbamate |
| 145 | 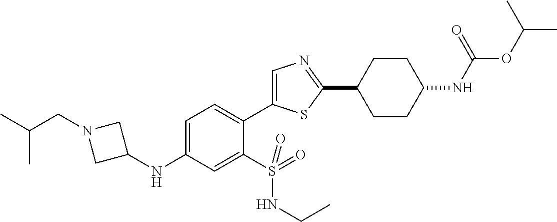 | isopropyl trans-N-[4-[5-[2- (ethylsulfamoyl)-4-[(1-isobutyl- azetidin-3- yl)amino]phenyl]thiazol-2- yl]cyclohexyl]carbamate |
| 146 | 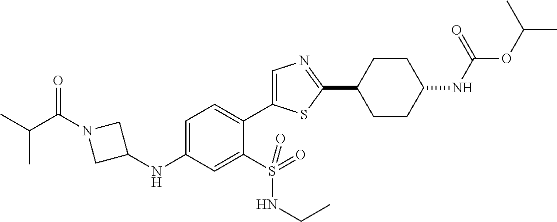 | isopropyl trans-N-[4-[5-[2- (ethylsulfamoyl)-4-[1-(2- methylpropanoyl)azetidin-3- yl]amino]phenyl]thiazol-2- yl]cyclohexyl]carbamate |
| 147 | 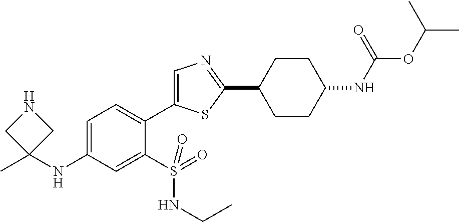 | isopropyl trans-N-[4-[5-[2- (ethylsulfamoyl)-4-[(3- methylazetidin-3- yl)amino]phenyl]thiazol-2- yl]cyclohexyl]carbamate |
| 148 | 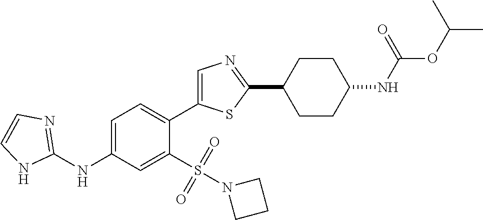 | isopropyl trans-N-[4-[5-[2- (azetidin-1-ylsulfonyl)-4-(1H- imidazol-2- ylamino)phenyl]thiazol-2- ylcyclohexyl]carbamate |
| 149 | 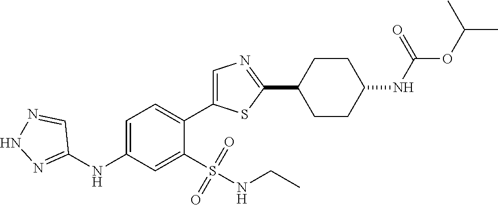 | isopropyl trans-N-[4-[5-[2- (ethylsulfamoyl)-4-(2H-triazol-4- ylamino)phenyl]thiazol-2- yl]cyclohexyl]carbamate |
| 150 | 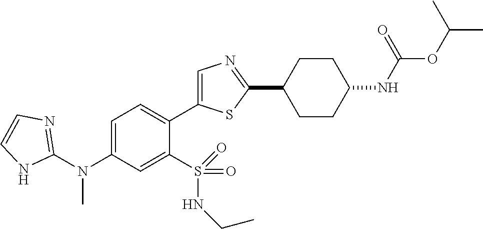 | isopropyl trans-N-[4-[5-[2- (ethylsulfamoy1)-4-[1H- imidazol-2- yl(methyl)amino]phenyl]thiazol- 2-yl]cyclohexyl]carbamate |
| 151 | 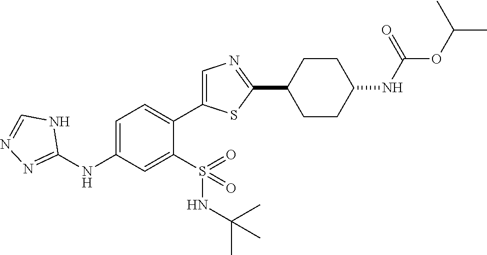 | isopropyl trans-N-[4-[5-[2-(tert- butylsulfamoyl)-4-(4H-1,2,4- triazol-3-ylamino)phenyl]thiazol- 2-yl]cyclohexyl]carbamate |
| 152 | 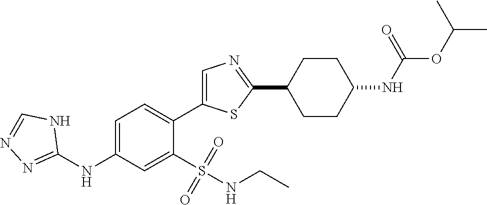 | isopropyl trans-N-[4-[5-[2- (ethylsulfamoyl)-4-(4H-1,2,4- triazol-3-ylamino)phenyl]thiazol- 2-yl]cyclohexyl]carbamate |
| 153 | 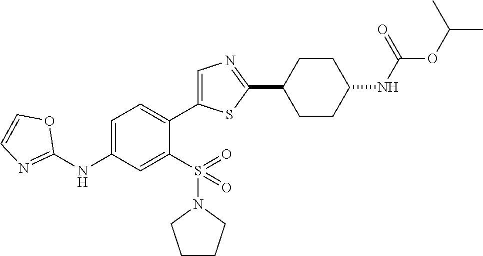 | isopropyl trans-[4-[5-[4-(oxazol- 2-ylamino)-2-pyrrolidin-1- ylsulfonyl-phenyl]thiazol-2- yl]cyclohexyl]carbamate |
| 154 | 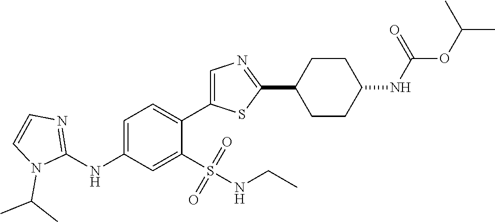 | isopropyl trans-N-[4-[5-[2- (ethylsulfamoyl)-4-[(1- isopropylimidazol-2- yl)amino]phenyl]thiazol-2- yl]cyclohexyl]carbamate |
| 155 | 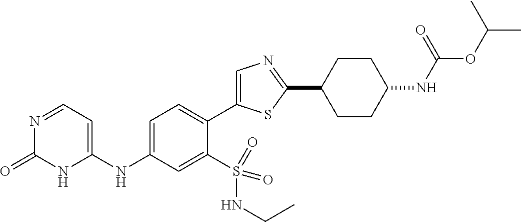 | isopropyl trans-N-[4-[5-[4-[(2- chloropyrimidin-4-yl)amino]-2- (ethylsulfamoyl)phenyl]thiazol- 2-yl]cyclohexyl]carbamate |
| 156 | 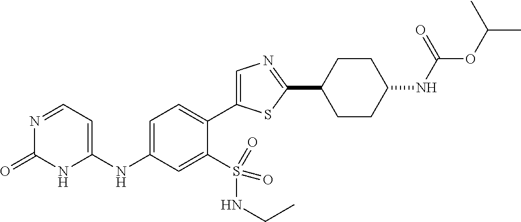 | isopropyl trans-N-[4-[5-[2- (ethylsulfamoyl)-4-[(2- hydroxypyrimidin-4- yl)amino]phenyl]thiazol-2- yl]cyclohexyl]carbamate |
| 157 | 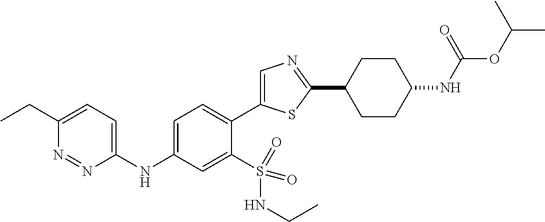 | isopropyl trans-N-[4-[5-[4-[(6- ethylpyridazin-3-yl)amino]-2- (ethylsulfamoyl)phenyl]thiazol- 2-yl]cyclohexyl]carbamate |
| 158 | 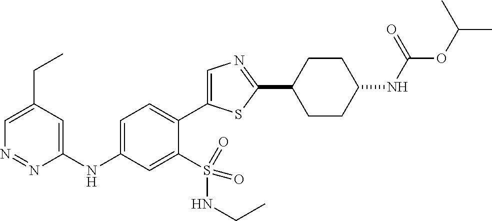 | isopropyl trans-N-[4-[5-[4-[(5- ethylpyridazin-3-yl)amino]-2- (ethylsulfamoyl)phenyl]thiazol- 2-yl]cyclohexyl]carbamate |
| 159 | 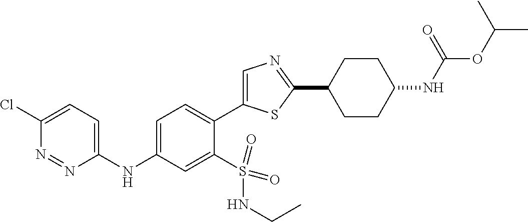 | isopropyl trans-N-[4-[5-[4-[(6- chloropyridazin-3-yl)amino]-2- (ethylsulfamoyl)phenyl]thiazol- 2-yl]cyclohexyl]carbamate |
| 160 | 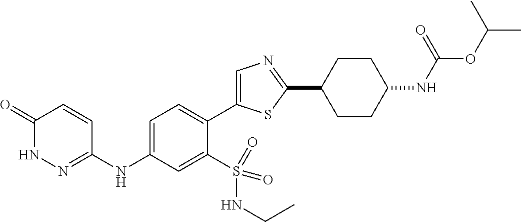 | isopropyl trans-N-[4-[5-[2- (ethylsulfamoyl)-4-[(6-oxo-1H- pyridazin-3- yl)amino]phenyl]thiazol-2- yl]cyclohexyl]carbamate |
| 161 | 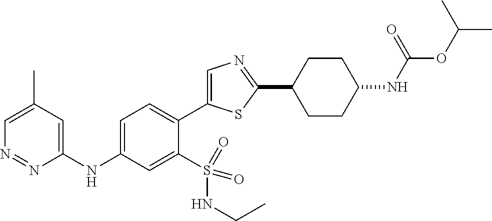 | isopropyl trans-N-[4-[5-[2- (ethylsulfamoyl)-4-[(5- methylpyridazin-3- yl)amino]phenyl]thiazol-2- yl]cyclohexyl]carbamate |
| 162 | 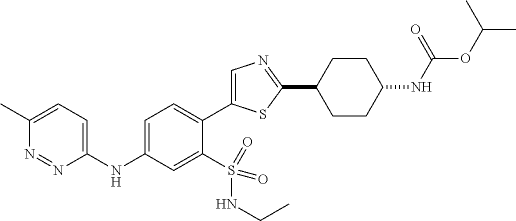 | isopropyl trans-N-[4-[5-[2- (ethylsulfamoy1)-4-[(6- methylpyridazin-3- yl)amino]phenyl]thiazol-2- yl]cyclohexyl]carbamate |
| 163 | 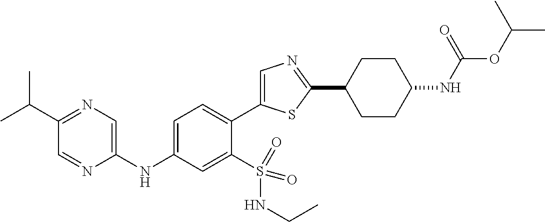 | isopropyl trans-N-[4-[5-[2- (ethylsulfamoyl)-4-[(5- isopropylpyrazin-2- yl)amino]phenyl]thiazol-2- yl]cyclohexyl]carbamate |
| 164 | 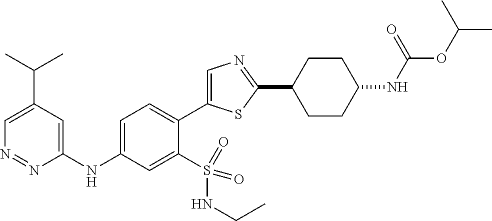 | isopropyl trans-N-[4-[5-[2- (ethylsulfamoyl)-4-[(5- isopropylpyridazin-3- yl)amino]phenyl]thiazol-2- yl]cyclohexyl]carbamate |
| 165 | 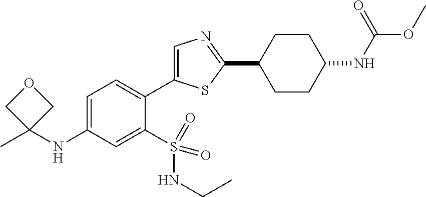 | methyl trans-N-[4-[5-[2- (ethylsulfamoyl)-4-[(3- methyloxetan-3- yl)amino]phenyl]thiazol-2- yl]cyclohexyl]carbamate |
| 166 | 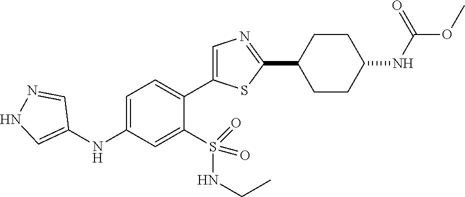 | methyl trans-N-[4-[5-[2- (ethylsulfamoyl)-4-[(1H-pyrazol- 4-yl)amino]phenyl]thiazol-2- yl]cyclohexyl]carbamate |
| 167 | 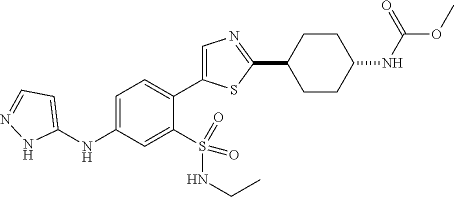 | methyl trans-N-[4-[5-[2- (ethylsulfamoyl)-4-[(1H-pyrazol- 5-yl)amino]phenyl]thiazol-2- yl]cyclohexyl]carbamate |
| 168 | 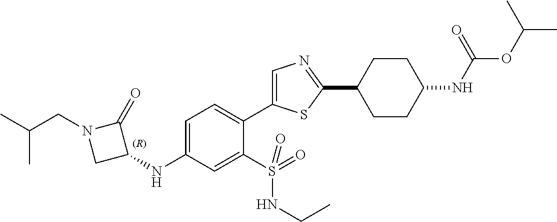 | isopropyl trans-N-[4-[5-[2- (ethylsulfamoyl)-4-[(3R)-1- isobutyl-2-oxo-azetidin-3- yl]amino]phenyl]thiazol-2- yl]cyclohexyl]carbamate |
| 169 | 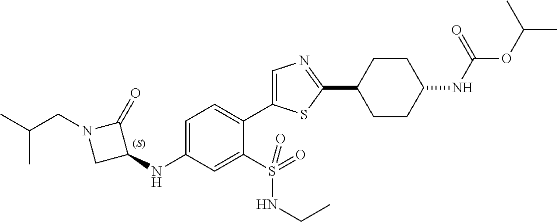 | isopropyl trans-N-[4-[5-[2- (ethylsulfamoy1)-4-[(3S)-1- isobutyl-2-oxo-azetidin-3- yl]amino]phenyl]thiazol-2- yl]cyclohexyl]carbamate |
| 170 | 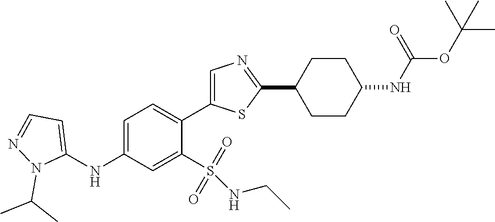 | tert-butyl trans-N-[4-[5-[2- (ethylsulfamoyl)-4-[(2- isopropylpyrazol-3- yl)amino]phenyl]thiazol-2- yl]cyclohexyl]carbamate |
| 171 | 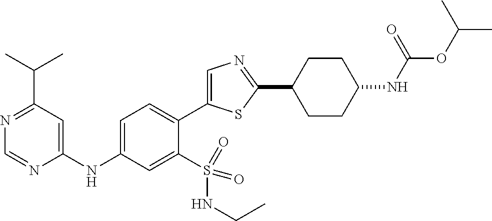 | isopropyl trans-N-[4-[5-[2- (ethylsulfamoyl)-4-[(6- isopropylpyrimidin-4- yl)amino]phenyl]thiazol-2- yl]cyclohexyl]carbamate |
| 172 | 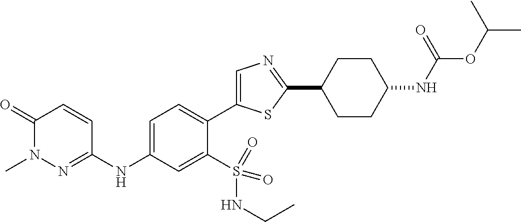 | isopropyl trans-N-[4-[5-[2- (ethylsulfamoyl)-4-[(1-methyl-6- oxo-pyridazin-3- yl)amino]phenyl]thiazol-2- yl]cyclohexyl]carbamate |
| 173 | 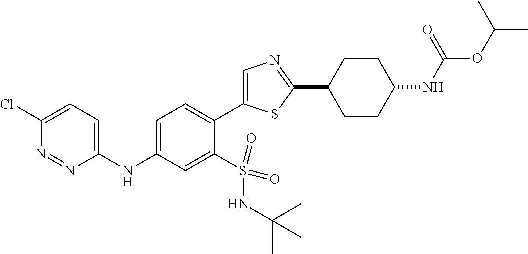 | isopropyl trans-N-[4-[5-[2-(tert- butylsulfamoyl)-4-[(6- chloropyridazin-3- yl)amino]phenyl]thiazol-2- yl]cyclohexyl]carbamate |
| 174 | 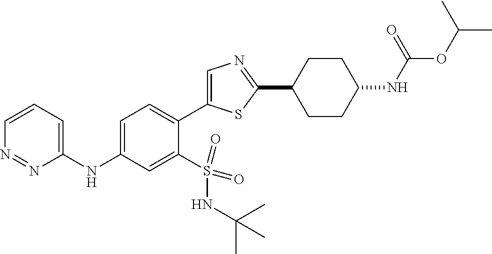 | isopropyl trans-N-[4-[5-[2-(tert- butylsulfamoyl)-4-(pyridazin-3- ylamino)phenyl]thiazol-2- yl]cyclohexyl]carbamate |

In some embodiments, a compound of the present application (e.g., a compound of any of the formulae or any individual compounds disclosed herein) is a pharmaceutically acceptable salt. In another embodiment, a compound of the present application (e.g., a compound of any of the formulae or any individual compounds disclosed herein) is a solvate. In another embodiment, a compound of the present application (e.g., a compound of any of the formulae or any individual compounds disclosed herein) is a hydrate.

The compounds of the present application may form salts which are also within the scope of this application. Reference to a compound of the Formula herein is understood to include reference to salts thereof, unless otherwise indicated.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Solvate” means solvent addition forms that contain either stoichiometric or non stoichiometric amounts of solvent. Some compounds or salts have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers. When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geometric isomers in the mixture.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

In some embodiments of the application, the compounds of the application are diastereomers. In some embodiments, the compounds are the syn diastereomer. In other embodiments, the compounds are the anti diastereomer.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diastereomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

It is also possible that the compounds of the application may exist in different tautomeric forms, and all such forms are embraced within the scope of the application. “Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-imine.

The application also comprehends isotopically-labeled compounds, which are identical to those recited in the each of the formulae described herein, but for the fact that one or more atoms are replaced by an atom having an atomic mass or mass number different from the atomic mass or mass number most commonly found in nature. Examples of isotopes that can be incorporated into compounds of the application include isotopes of hydrogen, carbon, nitrogen, fluorine, such as ³H, ¹¹C, ¹⁴C, ²H and ¹⁸F.

Compounds of the application that contain the aforementioned isotopes and/or other isotopes of other atoms are within the scope of the present application. Isotopically-labeled compounds of the present application, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are useful for their ease of preparation and detectability. ¹¹C and ¹⁸F isotopes are useful in PET (positron emission tomography). PET is useful in brain imaging. Further, substitution with heavier isotopes such as deuterium, i.e., ²H, can afford certain therapeutic advantages resulting from greater metabolic stability, for example, increased in vivo half-life or reduced dosage requirements and, hence, may be preferred in some circumstances. Isotopically labeled compounds of the application, can generally be prepared by carrying out the procedures disclosed in the Schemes and/or in the Examples described herein, by substituting a readily available isotopically labeled reagent for a non-isotopically labeled reagent. In some embodiments, the compounds of the application are not isotopically labelled.

### Methods for Preparing the Compounds

The compounds of the present application may be made by a variety of methods, including standard chemistry. Suitable synthetic routes are depicted in the Schemes given below.

The compounds of Formula I may be prepared by methods known in the art of organic synthesis as set forth in part by the following synthetic schemes. In the scheme described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of the compounds of the present application.

Those skilled in the art will recognize if a stereocenter exists in the compounds of Formula I. Accordingly, the present application includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds described herein may be made from commercially available starting materials or synthesized using known organic, inorganic, and/or enzymatic processes. The compounds of the present application can be prepared in a number of ways well known to those skilled in the art of organic synthesis. By way of example, the compounds of the present application can be synthesized using the methods described below, together with synthetic methods known in the art of synthetic organic chemistry, or variations thereon as appreciated by those skilled in the art. Preferred methods include but are not limited to those methods described below. The compounds of the present application (i.e., a compound of Formula I) can be synthesized by following the steps outlined in General Schemes and/or General Methods below. Starting materials are either commercially available or made by known procedures in the reported literature or as illustrated.

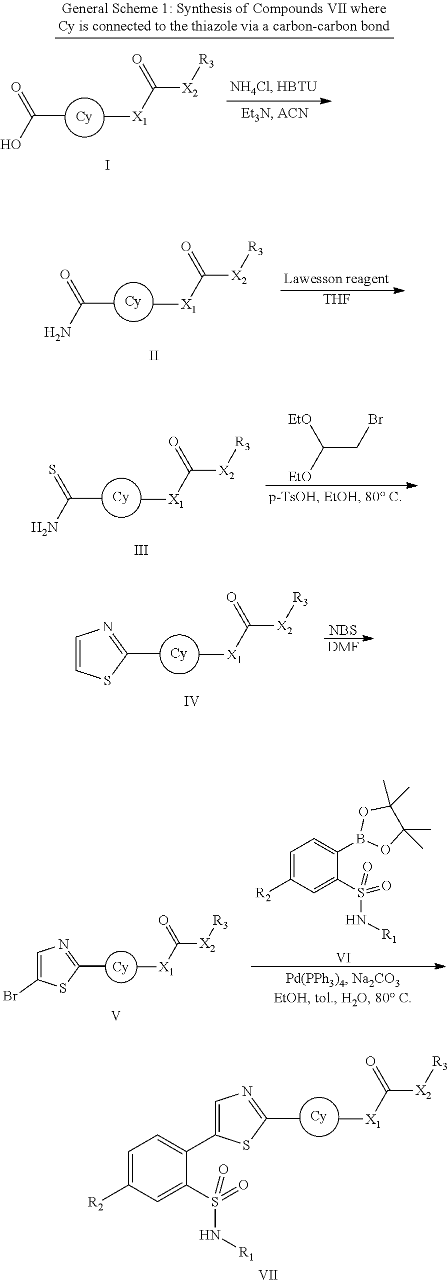

A carboxylic acid I is converted to the amide II using HBTU and ammonium chloride. The resulting amide is reacted with Lawesson's reagent in THF to provide the thioamide III. Cyclization with 2-bromo-1,1-diethoxyethane under acidic conditions gives the thiazole IV which is then brominated using N-bromosuccinimide. The bromothiazole V is then reacted under Suzuki coupling conditions with the boronate VI to give the MCT inhibitor VII or an intermediate which is further transformed using conventional chemistry protocols.

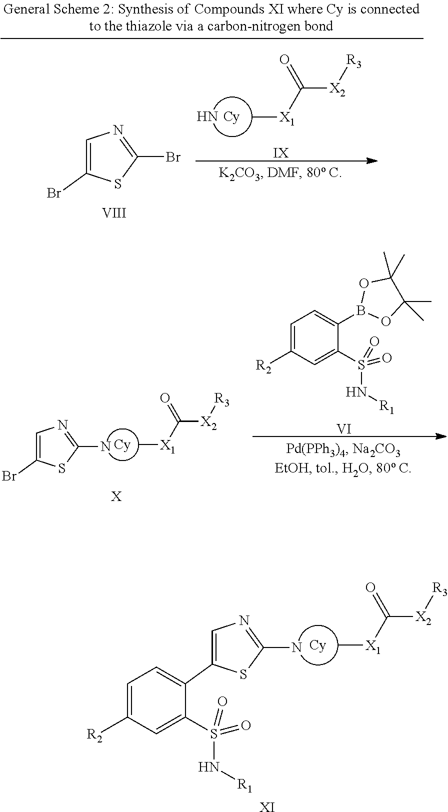

2,5-dibromothiazole (VIII) is reacted with a cycloalkylamine or heterocyclic amine (IX) in DMF to give the bromothiazole X. A Suzuki coupling with the boronate VI provides the MCT inhibitor XI or an intermediate which is further transformed using conventional chemistry protocols.

The following General Methods further illustrate the preparation of the compounds of the present application. Although certain reagents/materials and conditions are described in the General Methods, it is understood that equivalent reagents/materials and conditions may also be used.

| General Method | Reaction Name |
|---|---|
| A | Amide coupling reaction |
| B | Borate formation |
| C | Suzuki Reaction A |
| D | Suzuki Reaction B |
| E | Deprotection of tert-butylsulfonamide |
| F | Buchwald Reaction |
| G | Sulfamide formation |
| H | Reductive amination |
| I | Deprotection of SEM group |
| J | Hydrolysis reaction |
| K | Substitution Reaction |
| L | Sandmeyer reaction |

### General Method A

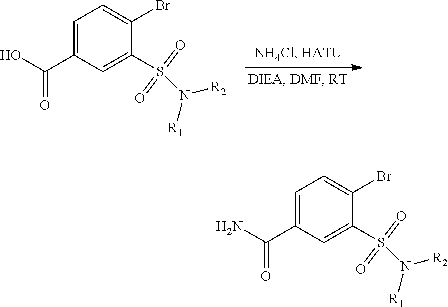

To a solution of carboxylic acid (1.0 eq.) and DIEA (15.0 eq.) in DMF may be added NH₄Cl (10.0 eq.) and HATU (1.3 eq.). The mixture may be stirred at 25° C. for 12 h, then poured into H₂O and extracted with EtOAc (3×). The combined organic layers may be washed with brine, dried over Na₂SO₄, filtered, and concentrated to yield the amide product.

### General Method B

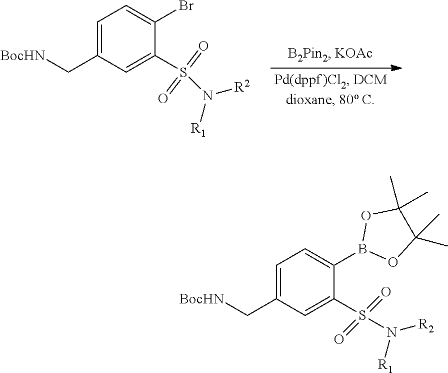

A mixture of Boc-protected amine (1.0 eq.), B₂Pin₂ (3.0 eq.), KOAc (3.0 eq.) and Pd(dppf)Cl₂·CH₂Cl₂ (0.1 eq.) in dioxane may be degassed and purged with N₂ (3×) and stirred at 80° C. for 12 h. The reaction mixture may be concentrated and the residue may be purified by prep-TLC (SiO₂, Petroleum ether/Ethyl acetate=3/1) to yield the borate product.

### General Method C

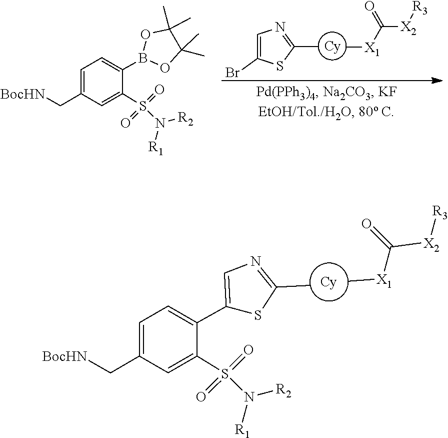

A mixture of borate (1.0 eq.), thiazole (1.2 eq.), Na₂CO₃ (3.0 eq.), Pd(PPh₃)₄ (0.1 eq.) and KF (3.0 eq.) in EtOH/H₂O/Tol. may be degassed and purged with N₂ (3×) and then stirred at 80° C. for 12 h. The reaction mixture may be concentrated and the residue may be purified by prep-TLC (SiO₂, Petroleum ether/Ethyl acetate=2/1) to yield the Suzuki Reaction A product.

### General Method D

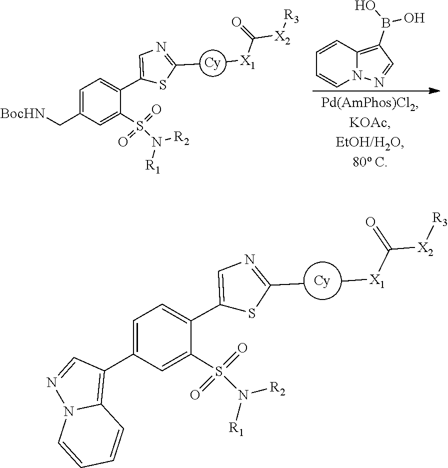

A mixture of brominated thiazole moiety (1.0 eq.), 3-(4,4,5,5-tetramethyl-1,3,2-dioxaborolan-2-yl)pyrazolo[1,5-a]pyridine (1.2 eq.), KOAc (3.0 eq.) and Pd(AmPhos)Cl₂ (0.1 eq.) in 1:0.25 EtOH/H₂O may be degassed and purged with N₂ (3×), and then stirred at 80° C. for 12 h. The reaction mixture may be concentrated and the residue may be purified by prep-HPLC (column: Welch Ultimate AQ-C18 150*30 mm*5 um; mobile phase: [water (0.1% TFA)-ACN]; B %: 50%-80%, 12 min) to yield the Suzuki Reaction B product.

### General Method E

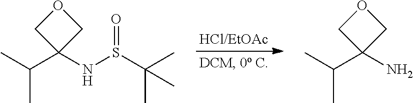

To a solution of N-(3-isopropyloxetan-3-yl)-2-methyl-propane-2-sulfinamide (1 eq.) in DCM may be added HCl/EtOAc (4 M, 4.4 eq.). The mixture may be stirred at 0° C. for 5 min, then the reaction mixture may be diluted with MTBE then the solid may be formed. The residue may be concentrated under reduced pressure to remove solvent at 0° C. Then the residue may be triturated with MTBE:Petroleum ether=1:1 and filtered, the filter cake may be collected to yield 3-isopropyloxetan-3-amine which may be used into the next step without further purification.

### General Method F

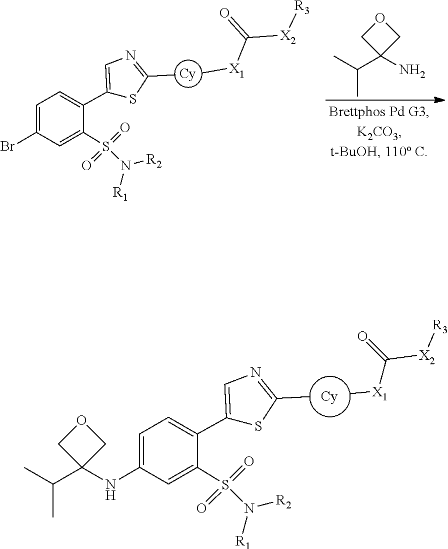

A mixture of 3-isopropyloxetan-3-amine (2 eq.), brominated thiazole moiety (1 eq.), K₂CO₃ (3 eq.) and BrettPhos Pd G3 (0.1 eq.) in t-BuOH may be stirred at 110° C. for 12 h under Ar atmosphere. The reaction mixture may be concentrated under reduced pressure, then the residue may be purified by prep-TLC (SiO₂, petroleum ether:ethyl acetate=2:1) and purified by prep-HPLC (column: YMC-Actus Triart C18 100*30 mm*5 um; mobile phase: [water (0.1% TFA)-ACN]; B %: 25%-60%, 10 min) to yield the Buchwald Reaction product.

### General Method G

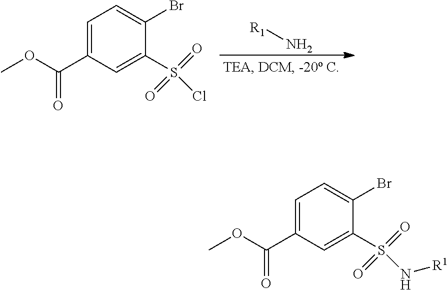

To a solution of TEA (3 eq.) in DCM may be added alkylamine (1.1 eq.) at −20° C. Then the methyl 4-bromo-3-chlorosulfonyl-benzoate (1.0 eq.) may be added batchwise. The mixture may be stirred at −20° C. for 5 min, then the reaction mixture may be quenched with HCl (1M, pH=2) and extracted with DCM, the organic layers may be dried over Na₂SO₄, filtered and concentrated under reduced pressure to yield the sulfamide formation product.

### General Method H

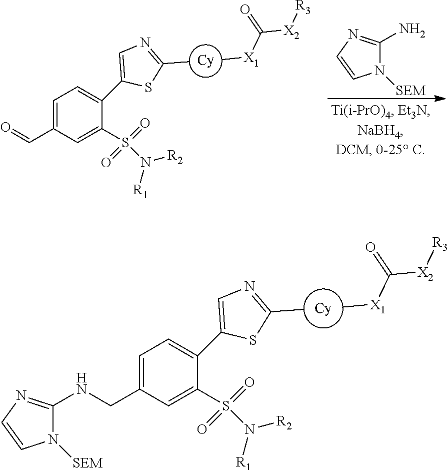

To a mixture of 1-(2-trimethylsilylethoxymethyl)imidazol-2-amine (1.5 eq.) and formyl-phenyl-thiazol moiety (1 eq.) in DCM may be added Ti(i-PrO)₄ (2 eq.) and Et₃N (0.1 eq.), then the mixture may be stirred at 25° C. for 16 h under N₂ atmosphere. The mixture may be added NaBH₄ (1.5 eq.) at 0° C., and stirred at 25° C. for 2 h. The reaction mixture may be quenched with H₂O at 25° C. and concentrated under reduced pressure. The residue may be purified by prep-TLC (SiO₂, petroleum ether:ethyl acetate=0:1) to yield the reductive amination product.

### General Method I

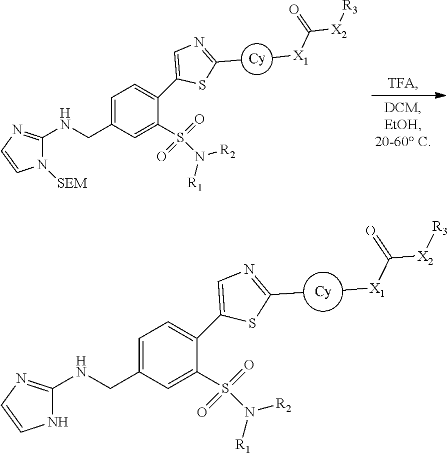

A mixture of SEM protected conjugated imidazole (1 eq.) in TFA and DCM may be stirred at 20° C. for 1 h. The reaction mixture may be concentrated under reduced pressure to remove DCM at 25° C. The residue may be diluted with EtOH and stirred at 60° C. for 3 h. The mixture may be concentrated under reduced pressure. The residue may be purified by prep-HPLC (TFA condition). (column: Welch Ultimate AQ-C18 150*30 mm*5 um; mobile phase: [water (0.1% TFA)-ACN]; B %: 18%-48%, 12 min) to yield the deprotection of SEM group product.

### General Method J

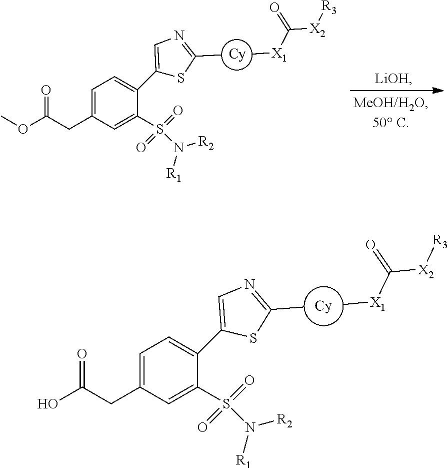

To a solution of ester (1 eq.) in 2:1 MeOH:H₂O, may be added LiOH (3 eq.). The mixture may be stirred at 50° C. for 2 h. The mixture may be concentrated under reduced pressure. The residue may be adjusted pH=2 with HCl (6M) and extracted with EtOAc (2×). The combined organic layers may be dried over Na₂SO₄, filtered and concentrated under reduced pressure to yield the hydrolysis reaction product.

### General Method K

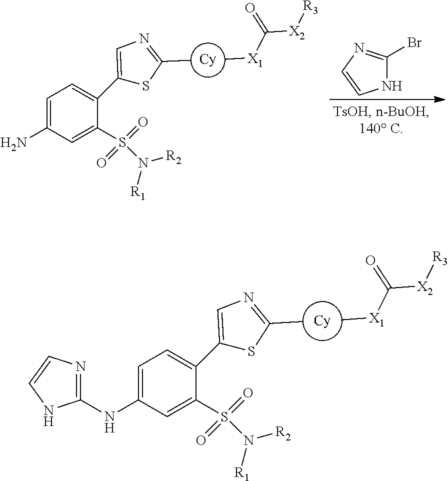

To a solution of amine (1 eq.) in n-BuOH may be added TsOH (3 eq.) and 2-bromo-1H-imidazole (5 eq.). The mixture may be stirred at 140° C. for 12 h. The reaction mixture may be concentrated under reduced pressure and purified by prep-HPLC (column: Waters Xbridge BEH C18 100*30 mm*10 um; mobile phase: [water (0.1% TFA)-ACN]; B %: 30%-90%, 10 min) to yield the substitution reaction product.

### General Method L

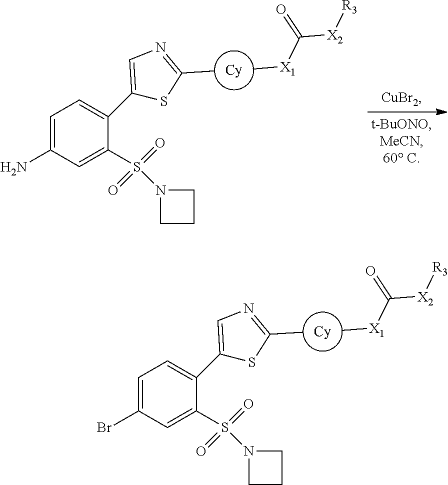

To a solution of amine (1 eq.) in MeCN may be added CuBr₂ (0.4 eq.) and tert-butyl nitrite (1.5 eq.). The mixture may be stirred at 60° C. for 1 h and concentrated. The residue may be diluted with H₂O and extracted with EtOAc (3×). The combined organic layers may be dried over Na₂SO₄, filtered and concentrated under reduced pressure to give a residue. The residue may be purified by prep-TLC (SiO₂, petroleum ether:ethyl acetate=1:2) to yield the Sandmeyer reaction product.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, such depiction also refers to the various neutral forms, cationic forms, and anionic forms of the compound. For another example, when a compound the present disclosure is depicted in an anionic form, such depiction also refers to various salts (e.g., sodium salt) of the anionic form of the compound.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

An “alkylene group” is a saturated aliphatic branched or straight-chain divalent hydrocarbon radical.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 6-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

The terms “heterocyclyl”, “heterocyclic ring”, and “heterocyclic group”, are used interchangeably herein, and means saturated or unsaturated non-aromatic 3-10 membered ring radical containing from 1 to 4 ring heteroatoms, which may be the same or different, selected from N, O, or S. It can be monocyclic, bicyclic or tricyclic (e.g., a fused or bridged bicyclic or tricyclic ring). Examples of include, but are not limited to, azetidinyl, morpholinyl, thiomorpholinyl, pyrrolidinonyl, pyrrolidinyl, piperidinyl, piperazinyl, hydantoinyl, valerolactamyl, oxiranyl, oxetanyl, dihydroimidazole, dihydrofuranyl, dihydropyranyl, dihydropyridinyl, dihydropyrimidinyl, dihydrothienyl, dihydrothiophenyl, dihydrothiopyranyl, tetrahydroimidazole, tetrahydrofuranyl, tetrahydropyranyl, tetrahydrothienyl, tetrahydropyridinyl, tetrahydropyrimidinyl, tetrahydrothiophenyl, and tetrahydrothiopyranyl. A heterocyclic ring optionally contains one or more double bonds and/or is optionally fused with one or more aromatic rings (for example, tetrahydronaphthyridine, indolinone, dihydropyrrolotriazole, imidazopyrimidine, quinolinone, dioxaspirodecane). Examples of 3-7 membered monocyclic heterocyclic ring include, but are not limited to, azetidinyl, morpholinyl, thiomorpholinyl, pyrrolidinonyl, pyrrolidinyl, piperidinyl, piperazinyl, hydantoinyl, valerolactamyl, oxiranyl, oxetanyl, dihydroimidazole, dihydrofuranyl, dihydropyranyl, dihydropyridinyl, dihydropyrimidinyl, dihydrothienyl, dihydrothiophenyl, dihydrothiopyranyl, tetrahydroimidazole, tetrahydrofuranyl, tetrahydropyranyl, tetrahydrothienyl, tetrahydropyridinyl, tetrahydropyrimidinyl, tetrahydrothiophenyl, and tetrahydrothiopyranyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated those compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who has (e.g., is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that doesn't respond or hasn't yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable prodrug, solvate, or salt thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable prodrug, solvate, or salt thereof, can or may also be used to prevent a relevant disease, condition, or disorder, or used to identify suitable candidates for such purposes.

As used herein, the term “preventing,” “prevent,” or “protecting against” describes reducing or eliminating the onset of the symptoms or complications of such disease, condition or disorder.

The terms “inhibiting”, “reducing”, or any variation of these terms in relation of MCT, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of MCT activity compared to its normal activity.

The term “disorder” is used in this disclosure to mean, and is used interchangeably with, the terms disease, condition, or illness, unless otherwise indicated.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches, and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable prodrugs, solvates, or salts thereof, are administered orally, nasally, transdermally, pulmonary, inhalationally, buccally, sublingually, intraperitoneally, subcutaneously, intramuscularly, intravenously, rectally, intrapleurally, intrathecally, and parenterally. In one embodiment, the compound is administered orally. One skilled in the art will recognise the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable prodrugs, solvates, or salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Pharmaceutical Compositions

The compounds disclosed therein are MCT modulators. The pharmaceutical composition of the present disclosure comprises one or more MCT modulators, or a pharmaceutically acceptable prodrug, solvate, or salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present teachings optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Administration and Dosage Forms

The precise amount of compound administered to provide an “effective amount” to the subject will depend on the mode of administration, the type, and severity of the disease, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the disclosure being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, the disclosed MCT modulators can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds described herein will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g., the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a MCT mediated disease using the disclosed MCT inhibitors for guidance.

The compounds or the corresponding pharmaceutical compositions taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the present teachings may be incorporated with excipient and used in the form of ingestible tablets, buccal tablets, troches, capsules, elixirs, suspensions, syrups, wafers, and the like.

Typically for parenteral administration, solutions of a compound of the present teachings can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

### EXAMPLES

### Example 1. Synthesis of the Compounds of the Present Disclosure

Compounds of the application can be prepared by methods known in the art. For example, compounds of the application may be synthesized according to the procedures described in WO 2020/186006.

### Example 2. Biological Activity of the Compounds of the Present Disclosure

The biological activity of the compounds of the present disclosure was determined utilizing the assays described herein.

### Intracellular Lactate Accumulation Assay

Intracellular lactate accumulation after a 2h treatment with compounds of the present invention was measured in Daudi (Burkitt Lymphoma) cells using the Lactate-Glo Assay (Promega).

### Procedure

Daudi cells were seeded at 7,500 cells per well in 150 μL of growth medium (RPMI-1640 with 2 mM GlutaMAX+10% FBS) in clear, 96-well, flat-bottom microplates, and incubated overnight (37° C., 5% CO₂). Compounds of the present invention were added using an INTEGRA VIAFLO 96 (8 point, 3-fold serial dilution, top concentration 1, 10, or 20 μM, 0.4% final DMSO concentration). After a 2h incubation (37° C., 5% CO₂) microplates containing cells were washed twice with 150 μL ice-cold PBS, resuspended in 25 μL ice-cold PBS containing 12.5 μL 0.6N HCl, and placed on a microplate shaker for 5 min to lyse the cells. Following lysis, 12.5 μL 1M Trizma base was added to neutralize the suspension, and microplates were returned to the microplate shaker for 1 min. Plates were sealed and stored at −20° C. until further processing. On the day of the assay, microplates were thawed for 45 min at room temperate and 50 μL Lactate Detection Reagent (prepared as specified by manufacturer) was added to each well. Microplates were placed on a plate shaker for 1 min and incubated at room temperature for 1h. Luminescence was measured using a BioTek Cytation 5 Multi-Mode Reader. Curve fitting and calculation of EC₅₀ was performed using GraphPad Prism.

### MCT1 Transport Inhibition Assay

Inhibition of MCT1 transport after treatment with compounds of the present invention was measured in MDCK-II cells that overexpress human MCT1 by quantifying the transport of 2-Thiophene-glycoxylic acid (TPGA) into cells using LC-MS/MS.

### Procedure

MDCK-II cells are seeded at 60,000 cells per well in growth medium (low-glucose DMEM+10% FBS) in 96-well trans-well membrane plates, and incubated overnight (37° C., 5% CO₂). Cells were co-transfected with mammalian expression constructs coding for MCT1 and CD147 at a 2:1 ratio or an empty vector control (GFP) and then incubated for 48h (37° C., 5% CO₂). Cells were washed 3 times with HBSS and cells were preincubated with inhibitors or vehicle control at room temperature in HBSS for 30 min with orbital shaking (60 rpm). HBSS was aspirated from the wells and replaced with HBSS with 25 mM Bis-Tris pH5.5+inhibitor or vehicle control+500 μM TPGA and incubated for 1 min at room temperature with orbital shaking (60 rpm). Both the apical and basolateral side of the trans-well insert were washed 4 times with ice-cold PBS. Cells were lysed with 60 μL of cell extraction solution, and the amount of TPGA in each well was quantified in triplicate by LC-MS/MS. The MCT1-mediated uptake rate was calculated using the following equation:

$$MCT 1 - mediated uptake rate ( pmol / min / {cm}^{2} ) = \frac{\begin{matrix}( Cellular accumulation in cells expressing MCT 1 ) - \\ ( Mean cellular accumulation in control cells )\end{matrix}}{Surface area \times incubation time}$$

Percent inhibition for each concentration of inhibitor tested was calculated using the following equation:

$$Percent inhibition = 100 - 100 \times \frac{{( MCT 1 - mediated uptake rate )}_{with inhibitor}}{{( MCT 1 - mediated uptake rate )}_{vehicle control}}$$

Curves were fit to the calculated percent inhibition for each inhibitor, and the IC₅₀ representing the concentration of inhibitor at which MCT1 is inhibited by 50% was calculated from this curve.

The biological activity of the compounds of the present application measured by the described assays above are shown in Table A below for LactateGlo EC₅₀ (“A” means <50 nM; “B” means ≥50 nM and <150 nM; “C” means ≥150 nM and <1000 nM; “D” means ≥1000 nM and <5000 nM; “E” means >5000 nM) and BioIVT MCT1 Transporter IC₅₀ (“+++++” means <50 nM; “++++” means ≥50 nM and <150 nM; “+++” means ≥150 nM and <1000 nM; “++” means ≥1000 nM and <5000 nM; “+” means >5000 nM). N/D=Not Determined.

TABLE A

| | | | BioIVT |
|---|---|---|---|
| | | | MCT1 |
| Ex. | | LactateGlo | Transporter |
| # | Structure | EC₅₀ (nM) | IC₅₀ (nM) |
| 121 | 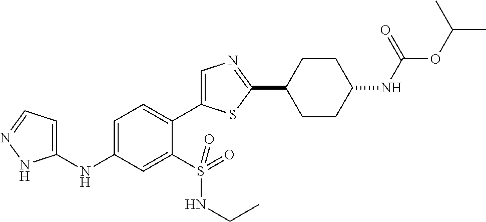 | A | N/D |
| 157 | 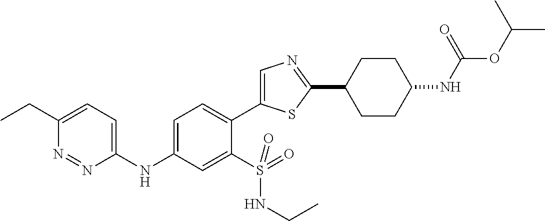 | A | ++++ |
| 175 | 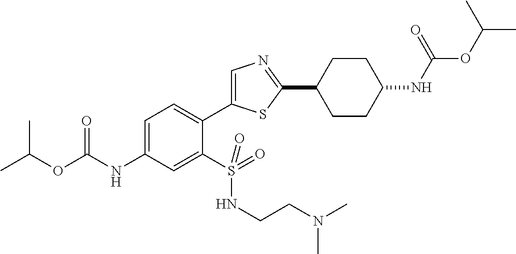 | D | N/D |
| 176 | 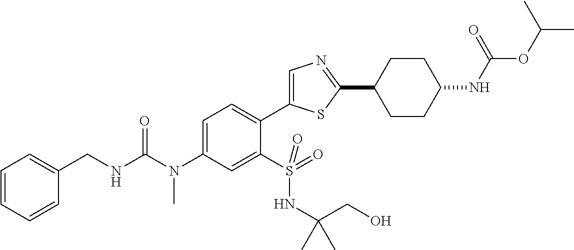 | E | N/D |
| | 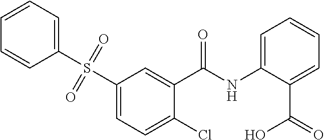 [SMILES: O=C(Nc1ccccc1C(=O)O)c1cc(S(=O)(=O)c2ccccc2)ccc1Cl] | N/D | ++++ |
| | BAY8002 (CAS No. 724440-27-1) | | |
| | 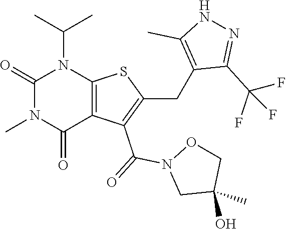 | N/D | ++++ |
| | AZD3965 (CAS No. 1448671-31-5) | | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A method of treating or preventing a disease or disorder associated with the abnormal expression or activity of monocarboxylate transporters (MCTs) or dependency on MCT expression of activity, wherein the method comprises administering to a subject in need thereof a compound of Formula I: 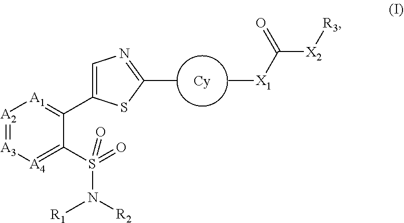
or a pharmaceutically acceptable prodrug, solvate, or salt thereof, wherein:
the thiazolyl ring 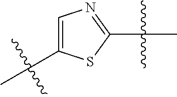 [SMILES: *c1cnc(*)s1]
is optionally substituted with F or Cl;
the ring Cy is C₃-C₇ cycloalkyl, bridged C₆-C₁₂ cycloalkyl, or saturated heterocyclyl comprising one or two 3- to 7-membered rings and 1-3 heteroatoms selected from N, O, and S, wherein the cycloalkyl or heterocyclyl moiety is optionally substituted with one or more groups selected from halogen, OH, CN, NH₂, C₁-C₄ alkyl, C₁-C₄ haloalkyl, C₁-C₄ alkoxy, and C₁-C₄ haloalkoxy;
X₁ is NR₈ or O, or, when X₁ is bonded to a nitrogen atom in the ring Cy, X₁ is absent;
X₂ is NR₈ or O;
R₁ is H or C₁-C₆ alkyl optionally substituted with halogen, OH, C₁-C₆ alkoxy, or C₆-C₁₀ aryloxy;
R₂ is H or C₁-C₆ alkyl optionally substituted with halogen, OH, C₁-C₆ alkoxy, or C₆-C₁₀ aryloxy;
or R₁ and R₂, together with the nitrogen atom to which they are attached, form a heterocyclyl comprising one or two 3- to 7-membered rings and 1-3 heteroatoms selected from N, O, and S;
R₃ is C₁-C₆ alkyl optionally substituted with one or more groups selected from halogen, OH, and CN, phenyl, CH₂-phenyl, C₃-C₇ cycloalkyl, CH₂—(C₃-C₇) cycloalkyl, heterocyclyl, or CH₂-heterocyclyl, wherein the heterocyclyl comprises one or two 3- to 7-membered rings and 1-3 heteroatoms selected from N, O, and S, wherein the cycloalkyl, phenyl, or heterocyclyl moiety is optionally substituted with one or more groups selected from halogen, OH, CN, C₁-C₄ alkyl, C₁-C₄ haloalkyl, C₁-C₄ alkoxy, and C₁-C₄ haloalkoxy;
A₁, A₂, A₃, and A₄ are each independently N or C(R₄);
each R₄ is independently H, halogen, CN, OH, N(R₆′)₂, C₁-C₆ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each Q is independently C₁-C₄ alkylene or O—(C₁-C₄) alkylene wherein the oxygen atom is bonded to the ring 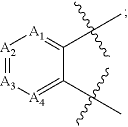
each T is independently C₁-C₄ alkoxy, OH, N(R₆)₂, N(R₅)C(═O)R₆, N(R₅)C(═O)OR₆, C(═O)N(R₆)₂, C(═O)OR₆, C(═O)R₆, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each R₅ is independently H or C₁-C₄ alkyl;
each R₆′ is independently H, C₁-C₆ alkyl optionally substituted with one or more R₇, C₁-C₆ haloalkyl, C₃-C₇ cycloalkyl, heterocyclyl comprising one or two 3- to 7-membered rings and 1-3 heteroatoms selected from N, O, and S, C₆-C₁₀ aryl, or heteroaryl comprising one or two 5- or 6-membered rings and 1-3 heteroatoms selected from N, O, and S, wherein the cycloalkyl, heterocyclyl, aryl, or heteroaryl moiety is optionally substituted with one or more R₉, wherein at least one R₆′ is not H;
or two R₆′ together with the atoms to which they are attached form a 3- to 10-membered heterocyclyl comprising 1-3 heteroatoms selected from N, O, and S, wherein the heterocyclyl is optionally substituted with one or more R₉;
each R₆ is independently H, C₁-C₆ alkyl optionally substituted with one or more R₇, C₁-C₆ haloalkyl, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
or two R₆ together with the atoms to which they are attached form a 3- to 10-membered heterocyclyl comprising 1-3 heteroatoms selected from N, O, and S, wherein the heterocyclyl is optionally substituted with one or more R₉;
each R₇ is independently N(R₈)₂, OR₈, C₆-C₁₀ aryl, or heteroaryl comprising one or two 5- or 6-membered rings and 1-3 heteroatoms selected from N, O, and S;
each R₈ is independently H or C₁-C₆ alkyl; and
each R₉ is independently oxo, halogen, OH, CN, NH₂, N(C₁-C₄ alkyl)₂, C₁-C₆ alkyl, N(C₁-C₄ alkyl)₂, C₁-C₆ haloalkyl, C₁-C₆ alkoxy, or C₁-C₆ haloalkoxy, wherein the C₁-C₆ alkyl is optionally substituted with one or more oxo, OH, O(C₁-C₄ alkyl), CN, NH₂, NH(C₁-C₄ alkyl), or N(C₁-C₄ alkyl)₂.

2. A method of treating or preventing a disease or disorder, wherein the method comprises administering to a subject in need thereof a compound of Formula I: 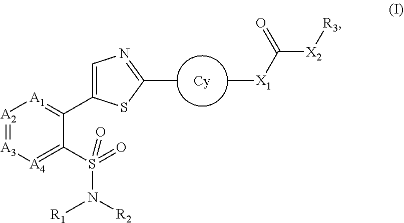
or a pharmaceutically acceptable prodrug, solvate, or salt thereof, wherein:
the thiazolyl ring 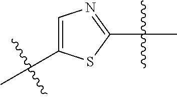 [SMILES: *c1cnc(*)s1]
is optionally substituted with F or Cl;
the ring Cy is C₃-C₇ cycloalkyl, bridged C₆-C₁₂ cycloalkyl, or saturated heterocyclyl comprising one or two 3- to 7-membered rings and 1-3 heteroatoms selected from N, O, and S, wherein the cycloalkyl or heterocyclyl moiety is optionally substituted with one or more groups selected from halogen, OH, CN, NH₂, C₁-C₄ alkyl, C₁-C₄ haloalkyl, C₁-C₄ alkoxy, and C₁-C₄ haloalkoxy;
X₁ is NR₈ or O, or, when X₁ is bonded to a nitrogen atom in the ring Cy, X₁ is absent;
X₂ is NR₈ or O;
R₁ is H or C₁-C₆ alkyl optionally substituted with halogen, OH, C₁-C₆ alkoxy, or C₆-C₁₀ aryloxy;
R₂ is H or C₁-C₆ alkyl optionally substituted with halogen, OH, C₁-C₆ alkoxy, or C₆-C₁₀ aryloxy;
or R₁ and R₂, together with the nitrogen atom to which they are attached, form a heterocyclyl comprising one or two 3- to 7-membered rings and 1-3 heteroatoms selected from N, O, and S;
R₃ is C₁-C₆ alkyl optionally substituted with one or more groups selected from halogen, OH, and CN, phenyl, CH₂-phenyl, C₃-C₇ cycloalkyl, CH₂—(C₃-C₇) cycloalkyl, heterocyclyl, or CH₂-heterocyclyl, wherein the heterocyclyl comprises one or two 3- to 7-membered rings and 1-3 heteroatoms selected from N, O, and S, wherein the cycloalkyl, phenyl, or heterocyclyl moiety is optionally substituted with one or more groups selected from halogen, OH, CN, C₁-C₄ alkyl, C₁-C₄ haloalkyl, C₁-C₄ alkoxy, and C₁-C₄ haloalkoxy;
A₁, A₂, A₃, and A₄ are each independently N or C(R₄);
each R₄ is independently H, halogen, CN, OH, N(R₆′)₂, C₁-C₆ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each Q is independently C₁-C₄ alkylene or O—(C₁-C₄) alkylene wherein the oxygen atom is bonded to the ring 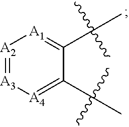
each T is independently C₁-C₄ alkoxy, OH, N(R₆)₂, N(R₅)C(═O)R₆, N(R₅)C(═O)OR₆, C(═O)N(R₆)₂, C(═O)OR₆, C(═O)R₆, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each R₅ is independently H or C₁-C₄ alkyl;
each R₆′ is independently H, C₁-C₆ alkyl optionally substituted with one or more R₇, C₁-C₆ haloalkyl, C₃-C₇ cycloalkyl, heterocyclyl comprising one or two 3- to 7-membered rings and 1-3 heteroatoms selected from N, O, and S, C₆-C₁₀ aryl, or heteroaryl comprising one or two 5- or 6-membered rings and 1-3 heteroatoms selected from N, O, and S, wherein the cycloalkyl, heterocyclyl, aryl, or heteroaryl moiety is optionally substituted with one or more R₉, wherein at least one R₆′ is not H;
or two R₆′ together with the atoms to which they are attached form a 3- to 10-membered heterocyclyl comprising 1-3 heteroatoms selected from N, O, and S, wherein the heterocyclyl is optionally substituted with one or more R₉;
each R₆ is independently H, C₁-C₆ alkyl optionally substituted with one or more R₇, C₁-C₆ haloalkyl, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
or two R₆ together with the atoms to which they are attached form a 3- to 10-membered heterocyclyl comprising 1-3 heteroatoms selected from N, O, and S, wherein the heterocyclyl is optionally substituted with one or more R₉;
each R₇ is independently N(R₈)₂, OR₈, C₆-C₁₀ aryl, or heteroaryl comprising one or two 5- or 6-membered rings and 1-3 heteroatoms selected from N, O, and S;
each R₈ is independently H or C₁-C₆ alkyl; and
each R₉ is independently oxo, halogen, OH, CN, NH₂, N(C₁-C₄ alkyl)₂, C₁-C₆ alkyl, N(C₁-C₄ alkyl)₂, C₁-C₆ haloalkyl, C₁-C₆ alkoxy, or C₁-C₆ haloalkoxy, wherein the C₁-C₆ alkyl is optionally substituted with one or more oxo, OH, O(C₁-C₄ alkyl), CN, NH₂, NH(C₁-C₄ alkyl), or N(C₁-C₄ alkyl)₂, and
wherein the compound of Formula I is administered in a therapeutically effective amount to modulate the activity of monocarboxylate transporters (MCTs).

3. A method of treating or preventing a disease or disorder, wherein the method comprises:
a. identifying a subject with an abnormal expression or activity of at least one MCT or dependency on MCT expression of activity; and
b. administering to the subject a compound of Formula I: 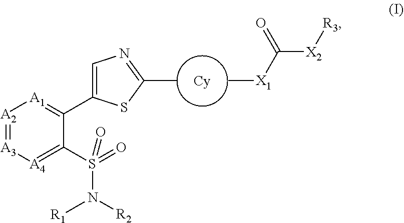
or a pharmaceutically acceptable prodrug, solvate, or salt thereof, wherein:
the thiazolyl ring 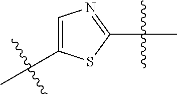 [SMILES: *c1cnc(*)s1]
is optionally substituted with F or Cl;
the ring Cy is C₃-C₇ cycloalkyl, bridged C₆-C₁₂ cycloalkyl, or saturated heterocyclyl comprising one or two 3- to 7-membered rings and 1-3 heteroatoms selected from N, O, and S, wherein the cycloalkyl or heterocyclyl moiety is optionally substituted with one or more groups selected from halogen, OH, CN, NH₂, C₁-C₄ alkyl, C₁-C₄ haloalkyl, C₁-C₄ alkoxy, and C₁-C₄ haloalkoxy;
X₁ is NR₈ or O, or, when X₁ is bonded to a nitrogen atom in the ring Cy, X₁ is absent;
X₂ is NR₈ or O;
R₁ is H or C₁-C₆ alkyl optionally substituted with halogen, OH, C₁-C₆ alkoxy, or C₆-C₁₀ aryloxy;
R₂ is H or C₁-C₆ alkyl optionally substituted with halogen, OH, C₁-C₆ alkoxy, or C₆-C₁₀ aryloxy;
or R₁ and R₂, together with the nitrogen atom to which they are attached, form a heterocyclyl comprising one or two 3- to 7-membered rings and 1-3 heteroatoms selected from N, O, and S;
R₃ is C₁-C₆ alkyl optionally substituted with one or more groups selected from halogen, OH, and CN, phenyl, CH₂-phenyl, C₃-C₇ cycloalkyl, CH₂—(C₃-C₇) cycloalkyl, heterocyclyl, or CH₂-heterocyclyl, wherein the heterocyclyl comprises one or two 3- to 7-membered rings and 1-3 heteroatoms selected from N, O, and S, wherein the cycloalkyl, phenyl, or heterocyclyl moiety is optionally substituted with one or more groups selected from halogen, OH, CN, C₁-C₄ alkyl, C₁-C₄ haloalkyl, C₁-C₄ alkoxy, and C₁-C₄ haloalkoxy;
A₁, A₂, A₃, and A₄ are each independently N or C(R₄);
each R₄ is independently H, halogen, CN, OH, N(R₆′)₂, C₁-C₆ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each Q is independently C₁-C₄ alkylene or O—(C₁-C₄) alkylene wherein the oxygen atom is bonded to the ring 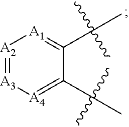
each T is independently C₁-C₄ alkoxy, OH, N(R₆)₂, N(R₅)C(═O)R₆, N(R₅)C(═O)OR₆, C(═O)N(R₆)₂, C(═O)OR₆, C(═O)R₆, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
each R₅ is independently H or C₁-C₄ alkyl;
each R₆′ is independently H, C₁-C₆ alkyl optionally substituted with one or more R₇, C₁-C₆ haloalkyl, C₃-C₇ cycloalkyl, heterocyclyl comprising one or two 3- to 7-membered rings and 1-3 heteroatoms selected from N, O, and S, C₆-C₁₀ aryl, or heteroaryl comprising one or two 5- or 6-membered rings and 1-3 heteroatoms selected from N, O, and S, wherein the cycloalkyl, heterocyclyl, aryl, or heteroaryl moiety is optionally substituted with one or more R₉, wherein at least one R₆′ is not H;
or two R₆′ together with the atoms to which they are attached form a 3- to 10-membered heterocyclyl comprising 1-3 heteroatoms selected from N, O, and S, wherein the heterocyclyl is optionally substituted with one or more R₉;
each R₆ is independently H, C₁-C₆ alkyl optionally substituted with one or more R₇, C₁-C₆ haloalkyl, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉;
or two R₆ together with the atoms to which they are attached form a 3- to 10-membered heterocyclyl comprising 1-3 heteroatoms selected from N, O, and S, wherein the heterocyclyl is optionally substituted with one or more R₉;
each R₇ is independently N(R₈)₂, OR₈, C₆-C₁₀ aryl, or heteroaryl comprising one or two 5- or 6-membered rings and 1-3 heteroatoms selected from N, O, and S;
each R₈ is independently H or C₁-C₆ alkyl; and
each R₉ is independently oxo, halogen, OH, CN, NH₂, N(C₁-C₄ alkyl)₂, C₁-C₆ alkyl, N(C₁-C₄ alkyl)₂, C₁-C₆ haloalkyl, C₁-C₆ alkoxy, or C₁-C₆ haloalkoxy, wherein the C₁-C₆ alkyl is optionally substituted with one or more oxo, OH, O(C₁-C₄ alkyl), CN, NH₂, NH(C₁-C₄ alkyl), or N(C₁-C₄ alkyl)₂.

4. The method of any one of claims 1 to 3, wherein the MCT is MCT1.

5. The method of any one of claims 1 to 3, wherein the MCT is MCT4.

6. The method of any one of claims 1 to 5, wherein the expression or activity of the MCT is increased.

7. The method of any one of claims 1 to 5, wherein the expression or activity of the MCT is decreased.

8. The method of any one of claims 1 to 7, wherein the expression or activity of MCT1 is increased.

9. The method of any one of claims 1 to 8, wherein the expression or activity of MCT4 is decreased.

10. The method of any one of claims 1 to 9, wherein the MCT activity of the compounds of Formula I is assessed using a lactate transporter assay.

11. The method of any one of claims 1 to 10, wherein the disease or disorder is cancer.

12. The method of claim 11, wherein the cancer is a MCT1 high-expressing cancer.

13. The method of claim 11 or claim 12, wherein the cancer is a lymphoma, myeloma, or a solid tumor.

14. The method of any one of claims 1 to 13, wherein the compound of Formula I inhibits the activity of MCT1 with an IC₅₀ from about 5 nM to about 1000 nM.

15. The method of any one of the preceding claims, wherein the ring Cy is C₃-C₇ cycloalkyl or saturated heterocyclyl comprising one or two 3- to 7-membered rings and 1-3 heteroatoms selected from N, O, and S, wherein the cycloalkyl or heterocyclyl is optionally substituted with one or more groups selected from halogen, OH, CN, NH₂, C₁-C₄ alkyl, C₁-C₄ haloalkyl, C₁-C₄ alkoxy, and C₁-C₄ haloalkoxy.

16. The method of any one of the preceding claims, wherein A₁, A₂, A₃, and A₄ are each C(R₄).

17. The method of any one of claims 1 to 15, wherein one, two, or three of A₁, A₂, A₃, and A₄ are N.

18. The method of any one of the preceding claims, wherein X₁ is NR₈ and X₂ is NR₈.

19. The method of any one of claims 1 to 17, wherein X₁ is NR₈ and X₂ is O.

20. The method of any one of claims 1 to 17, wherein X₁ is O and X₂ is NR₈.

21. The method of any one of claims 1 to 17, wherein X₁ is absent and X₂ is NR₈.

22. The method of any one of claims 1 to 17, wherein X₁ is absent and X₂ is O.

23. The method of any one of the preceding claims, wherein R₃ is C₁-C₆ alkyl optionally substituted with one or more groups selected from halogen, OH, and CN.

24. The method of any one of claims 1 to 22, wherein R₃ is phenyl or CH₂-phenyl, wherein the phenyl moiety is optionally substituted with one or more groups selected from halogen, OH, CN, C₁-C₄ alkyl, C₁-C₄ haloalkyl, C₁-C₄ alkoxy, and C₁-C₄ haloalkoxy.

25. The method of any one of claims 1 to 22, wherein R₃ is C₃-C₇ cycloalkyl or CH₂—(C₃-C₇) cycloalkyl, wherein the cycloalkyl moiety is optionally substituted with one or more groups selected from halogen, OH, CN, C₁-C₄ alkyl, C₁-C₄ haloalkyl, C₁-C₄ alkoxy, and C₁-C₄ haloalkoxy.

26. The method of any one of claims 1 to 22, wherein R₃ is heterocyclyl or CH₂-heterocyclyl, wherein the heterocyclyl moiety is optionally substituted with one or more groups selected from halogen, OH, CN, C₁-C₄ alkyl, C₁-C₄ haloalkyl, C₁-C₄ alkoxy, and C₁-C₄ haloalkoxy.

27. The method of any one of the preceding claims, wherein R₁ is H.

28. The method of any one of claims 1 to 26, wherein R₁ is C₁-C₆ alkyl optionally substituted with halogen, OH, C₁-C₆ alkoxy, or C₆-C₁₀ aryloxy.

29. The method of any one of the preceding claims, wherein R₂ is H.

30. The method of any one of claims 1 to 28, wherein R₂ is C₁-C₆ alkyl optionally substituted with halogen, OH, C₁-C₆ alkoxy, or C₆-C₁₀ aryloxy.

31. The method of any one of claims 1 to 28, wherein R₁ and R₂, together with the nitrogen atom to which they are attached, form a heterocyclyl.

32. The method of any one of the preceding claims, wherein each R₄ is H.

33. The method of any one of claims 1 to 31, wherein at least one R₄ is halogen, CN, OH, N(R₆′)₂, C₁-C₄ alkoxy, C(═O)N(R₆)₂, C(═O)OR₆, C(═O)R₆, Q-T, C₆-C₁₀ aryl, or heteroaryl comprising one or two 5- or 6-membered rings and 1-3 heteroatoms selected from N, O, and S, wherein the aryl or heteroaryl moiety is optionally substituted with one or more R₉.

34. The method of any one of claims 1 to 14, wherein the compound is of Formula Ia, Ib, Ic, Id, Ie, If, or Ig: 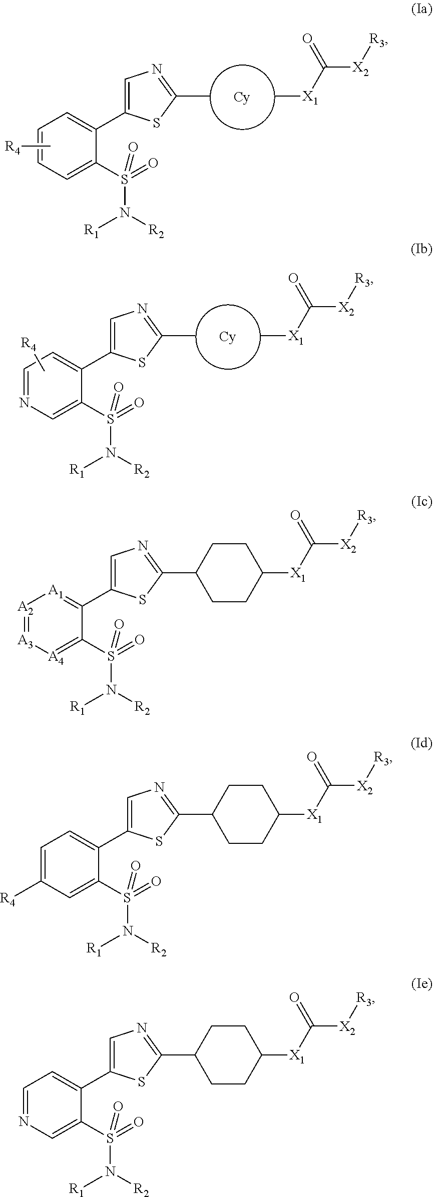 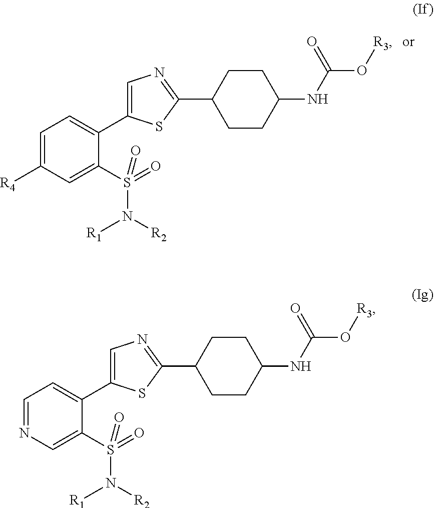
or a pharmaceutically acceptable salt or solvate thereof.

35. A method of treating or preventing a disease or disorder associated with the abnormal expression or activity of monocarboxylate transporters (MCTs) or dependency on MCT expression of activity, wherein the method comprises administering to a subject in need thereof a compound of Table 1, or a pharmaceutically acceptable prodrug, solvate, or salt thereof.

36. The method of any one of claims 1 to 14, wherein the compound is 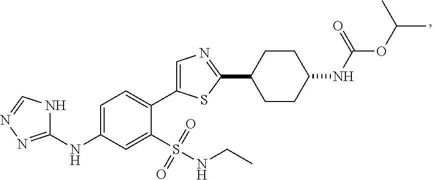
or a pharmaceutically acceptable salt or solvate thereof.

37. The method of any one of claims 1 to 14, wherein the compound is 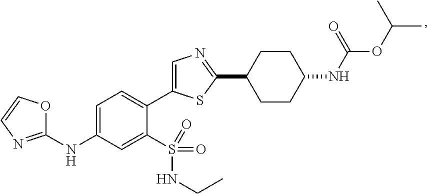
or a pharmaceutically acceptable salt or solvate thereof.

38. The method of an one of claims 1 to 14, wherein the compound is 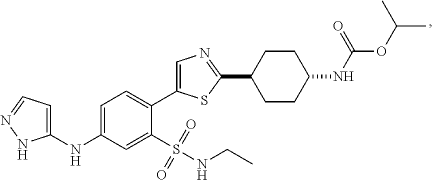
or a pharmaceutically acceptable salt or solvate thereof.

39. The method of any one of the preceding claims, wherein the compound of Formula I is comprised in a pharmaceutical composition comprising a pharmaceutically acceptable carrier or diluent and a compound of Formula I or a pharmaceutically acceptable prodrug, solvate, or salt thereof.

40. Use of a compound or a pharmaceutically acceptable prodrug, solvate, or salt thereof of Formula I, or a pharmaceutical composition thereof, in the manufacture of a medicament for treating or preventing a disease or disorder associated with the abnormal expression or activity of monocarboxylate transporters (MCTs), or dependency on MCT expression of activity.

41. Use of a compound or a pharmaceutically acceptable prodrug, solvate, or salt thereof of Formula I, or a pharmaceutical composition thereof, in the manufacture of a medicament for treating or preventing a disease or disorder, and
wherein the compound is administered in a therapeutically effective amount to modulate the activity of monocarboxylate transporters (MCTs).

42. Use of a compound or a pharmaceutically acceptable prodrug, solvate, or salt thereof of Formula I, or a pharmaceutical composition thereof, in the manufacture of a medicament for treating or preventing a disease or disorder comprising:
a. identifying a subject with an abnormal expression or activity of at least one MCT, or dependency on MCT expression of activity; and
b. administering to the subject a compound or a pharmaceutically acceptable prodrug, solvate, or salt thereof of Formula I, or a pharmaceutical composition thereof.

43. Use of a compound or a pharmaceutically acceptable prodrug, solvate, or salt thereof of Formula I, or a pharmaceutical composition thereof, for treating or preventing a disease or disorder associated with the abnormal expression or activity of monocarboxylate transporters (MCTs), or dependency on MCT expression of activity.

44. Use of a compound or a pharmaceutically acceptable prodrug, solvate, or salt thereof of Formula I, or a pharmaceutical composition thereof, for treating or preventing a disease or disorder, and
wherein the compound is administered in a therapeutically effective amount to modulate the activity of monocarboxylate transporters (MCTs).

45. Use of a compound or a pharmaceutically acceptable prodrug, solvate, or salt thereof of Formula I, or a pharmaceutical composition thereof, for treating or preventing a disease or disorder comprising:
a. identifying a subject with an abnormal expression or activity of at least one MCT, or dependency on MCT expression of activity; and
b. administering to the subject a compound or a pharmaceutically acceptable prodrug, solvate, or salt thereof of Formula I, or a pharmaceutical composition thereof.

46. A compound or a pharmaceutically acceptable prodrug, solvate, or salt thereof of Formula I, or a pharmaceutical composition thereof, for use in treating or preventing a disease or disorder associated with the abnormal expression or activity of monocarboxylate transporters (MCTs), or dependency on MCT expression of activity.

47. A compound or a pharmaceutically acceptable prodrug, solvate, or salt thereof of Formula I, or a pharmaceutical composition thereof, for use in treating or preventing a disease or disorder, and
wherein the compound is administered in a therapeutically effective amount to modulate the activity of monocarboxylate transporters (MCTs).

48. A compound or a pharmaceutically acceptable prodrug, solvate, or salt thereof of Formula I, or a pharmaceutical composition thereof, for use in treating or preventing a disease or disorder comprising:
a. identifying a subject with an abnormal expression or activity of at least one MCT, or dependency on MCT expression of activity; and
b. administering to the subject a compound or a pharmaceutically acceptable prodrug, solvate, or salt thereof of Formula I, or the pharmaceutical composition thereof.

46. A compound or a pharmaceutically acceptable prodrug, solvate, or salt thereof of Formula I, or a pharmaceutical composition thereof, for use in treating or preventing a disease or disorder associated with the abnormal expression or activity of monocarboxylate transporters (MCTs), or dependency on MCT expression of activity.

47. A compound or a pharmaceutically acceptable prodrug, solvate, or salt thereof of Formula I, or a pharmaceutical composition thereof, for use in treating or preventing a disease or disorder, and
wherein the compound is administered in a therapeutically effective amount to modulate the activity of monocarboxylate transporters (MCTs).

48. A compound or a pharmaceutically acceptable prodrug, solvate, or salt thereof of Formula I, or a pharmaceutical composition thereof, for use in treating or preventing a disease or disorder comprising:
a. identifying a subject with an abnormal expression or activity of at least one MCT, or dependency on MCT expression of activity; and
b. administering to the subject a compound or a pharmaceutically acceptable prodrug, solvate, or salt thereof of Formula I, or the pharmaceutical composition thereof.
